(19) United States
(12) Patent Application Publication
(10) Pub. No.: US 2024/0082212 A1
(43) Pub. Date: Mar. 14, 2024

(54) USE OF MDMA FOR TREATMENT OF STRESS-RELATED DISORDERS

(71) Applicant: ATAI Life Sciences AG, Berlin (DE)

(72) Inventors: Glenn Short, Scituate, MA (US); Carrie Bowen, Boston, MA (US); Srinivas Rao, Encinitas, CA (US)

(21) Appl. No.: 18/244,078

(22) Filed: Sep. 8, 2023

Related U.S. Application Data
(60) Provisional application No. 63/405,158, filed on Sep. 9, 2022.

(51) Int. Cl. A61K 31/36; A61P 21/00; A61P 25/00

(52) U.S. Cl. CPC A61K 31/36; A61P 21/00; A61P 25/00

(57) ABSTRACT

Methods of treating a stress-related disease or disorder such as PTSD, decreasing side effects of 3,4-methylenedioxy-methamphetamine (MDMA), inducing neurite outgrowth, inducing structural neuroplasticity, or increasing brain-derived neurotrophic factor (BDNF) levels, comprising administering to a subject effective amounts of a composition comprising R(−)-3,4-methylenedioxymethamphetamine (R(−)-MDMA) are disclosed. Also disclosed are pharmaceutical compositions comprising R(−)-MDMA and a pharmaceutically acceptable carrier.

### RELATED APPLICATIONS

This application is related to and claims the benefit of priority to U.S. Provisional Patent Application Ser. No. 63/405,158, filed Sep. 9, 2022, and which is incorporated herein by reference in its entirety.

### FIELD

The disclosure relates to the use of psychedelic compounds for the treatment of stress-related disorders, activating 5-HT receptors, as well as inducing beneficial neural activity and effects. In particular the disclosure provides for compositions, methods and the use of R(−)-3,4-methylenedioxymethamphetamine (R(−)-MDMA) in such methods, and which can reduce the frequency and/or severity of common side effects associated with MDMA (i.e., racemic MDMA and/or (S)(+)-MDMA).

### BACKGROUND

3,4-methylenedioxymethamphetamine (MDMA) is a psychoactive compound that affects mood, perception, and increases prosocial feelings and behaviors. It is a ring-substituted phenethylamine possessing a complex pharmacological profile that is dominated by its effects as a monoamine releaser and reuptake inhibitor. Its prominent serotonergic effects differentiate it from amphetamine and methamphetamine, which primarily act via dopaminergic and norepinephrine mechanisms of action. MDMA is considered the prototype for compounds referred to as entactogens, which means “to touch within” due to the induction of feelings of empathy, and sociability. MDMA produces subjective effects that are unlike any of the classical psychostimulants or hallucinogens and is one of the few compounds capable of reliably producing prosocial behavioral states.

Racemic MDMA possesses a complex pharmacological profile as a result of enhanced triple monoamine (serotonin, norepinephrine, and dopamine) release coupled with inhibition of reuptake, blockade of vesicular storage of monoamines, inhibition of neurotransmitter oxidation by MAO-A, and a reversal of 5-HT transport into the neuron. These effects result in a net increase of monoamines in the synaptic cleft and prolong the duration of monoaminergic neurotransmission. MDMA also affects hormone secretion, promoting the release of oxytocin and arginine vasopressin (AVP).

MDMA produces anxiolytic and prosocial effects through the release of monoaminergic neurotransmitters. Subjective effects of MDMA can include increased compassion for self and others, reduced defenses and fear of emotional injury, and making unpleasant memories less disturbing while enhancing communication and capacity for introspection. Collectively, these factors provide the opportunity for a corrective emotional experience in the context of therapy. However, MDMA has adverse events observed in clinical and nonclinical studies, such as cardiovascular effects, hyperthermia and neurotoxicity that may limit its clinical viability.

MDMA has two stereoisomers S(+)-MDMA and R(−)-MDMA. The enantiomers have been shown to impact the monoaminergic targets of MDMA differently, resulting in pharmacological activity of S(+)-MDMA resembling those of psychostimulants, including increases in motor activity and euphoria, and R(−)-MDMA pharmacologic activity inducing effects closer to classical psychedelics, such as alteration in perception and ego-dissolution. The differing pharmacological and toxicological profile of the enantiomers indicate that R(−)-MDMA can provide an improved therapeutic index which could provide a compound that has the therapeutic effects of racemic MDMA but with a reduced side effect profile.

Post-traumatic stress disorder (PTSD) is a serious, chronic, life-threatening psychiatric disorder. Symptoms include recurring and intrusive negative thoughts or recollections of the traumatic event, cognitive disruption, hyperarousal to event related cues, and avoidance behaviors that persist for longer periods than a month after experiencing a traumatic event. PTSD results in the overall reduction in the quality of life for individuals with PTSD leading to disability and can affect physical health with manifestation of other comorbidities such as cardiovascular disease (obesity, hypertension), concomitant mental health conditions and suicidality.

There are only two pharmacotherapies currently approved for the treatment of PTSD, sertraline (Zoloft®) and paroxetine (Paxil®). These pharmacotherapeutics are serotonin reuptake inhibitors (SSRIs), which, like racemic MDMA, increase the level of serotonin in the synaptic cleft. Sertraline and paroxetine have demonstrated moderate efficacy in reducing PTSD symptoms, but rarely result in full disorder remission and have problematic side effects and generally require long-term and/or consistent use to maintain effectiveness. Clinical practice guidelines recommend psychotherapy as the first-line treatment for PTSD because of the low response rate to existing pharmacotherapy.

Given the overall ineffectiveness of current pharmacotherapy and the limitations of patient access to trauma-focused psychotherapy for treatment of PTSD, novel treatments are needed. PTSD remains a mental health disorder of high unmet medical need. Because R(−)-MDMA has differing pharmacological and toxicological profiles compared with racemic MDMA and S(+)-MDMA, R(−)-MDMA can provide an improved therapeutic index for stress-related disorders such as PTSD, offering the therapeutic effects of racemic MDMA but with a reduced side effect profile.

### SUMMARY

The disclosure exploits the different pharmacological and toxicological profiles, and thus the different functional characteristics possessed by R(−)-MDMA and S(+)-MDMA, as well as racemic MDMA. Specifically, and as detailed herein, R(−)-MDMA can induce neurite growth increasing neural plasticity and has many of the functional properties desirable for treating stress-related diseases or disorders such as PTSD. Further, methods and uses for treatment that comprise R(−)-MDMA, as well as particular formulations, delivery routes, and dosing regimen can provide for additional beneficial outcomes including, for example, a reduction in the occurrence and severity of negative side effects that are associated with administration of racemic and/or S(+)-MDMA in subjects.

In one embodiment, the disclosure provides methods of treating a stress-related disease or disorder in a subject including administering to the subject a therapeutically effective amount of a composition including R(−)-3,4-methylenedioxymethamphetamine (R(−)-MDMA) to lessen avoidance behavior in the subject. In some further embodiments, the methods comprise treating stress-related disease or disorder including, for example, post-traumatic stress disorder, social anxiety, autism spectrum disorder, substance use disorder, depression, anxiety disorder, anxiety with life-threatening disease, personality disorder including narcistic or antisocial personality disorder, schizophrenia, obsessive compulsive disorder, gambling, aberrant sexual behavior, an auditory disorder, an additive disorder (e.g., substance use disorder such as alcohol abuse, substance abuse, smoking), an eating disorder (anorexia nervosa, bulimia nervosa, binge eating disorder, etc.), an auditory disorder, an impulsive disorder (e.g., attention deficit hyperactivity disorder (ADHD), attention deficit disorder (ADD), or Tourette's syndrome), enhancing of a psychotherapy and/or enhancing or inducing feelings of well-being connectivity, trust, love, empathy, openness, and pro-sociality.

In an embodiment, the stress-related disease or disorder is mood/depressive disorder, bipolar disorder, anxiety disorder, psychotic or delirium disorder, schizophrenia, schizoaffective disorder, personality disorder, abuse or neglect disorder, tic disorder, neurocognitive disorder, neurodevelopmental disorder, learning disorder, disruptive mood regulation disorder, intermittent explosive disorder, antisocial personality disorder, conduct disorder, behavioral and psychological symptoms of dementia, depression, treatment resistant depression, anxiety, post-traumatic stress disorder (PTSD), or any combination. In an embodiment, the stress-related disease or disorder is PTSD.

In an embodiment, R(−)-MDMA has antidepressant and anxiolytic effects.

In an embodiment, said therapeutically effective amount includes between about 1 mg/kg and 20 mg/kg R(−)-MDMA. In an embodiment, the therapeutically effective amount includes between about 25 mg and 350 mg R(−)-MDMA. In an embodiment, the therapeutically effective amount includes about 5 mg/kg R(−)-MDMA. In an embodiment, the administering is by intracutaneous, subcutaneous, intravenous, intraarterial, intradermal, transdermal, oral, sublingual, buccal, or nasal route of administration. In an embodiment, R(−)-MDMA is administered as a single dose. In an embodiment, R(−)-MDMA is administered in repeated doses.

In an embodiment, the method can also include administering a second therapeutic agent. In an embodiment, the second therapeutic agent is a selective serotonin reuptake inhibitor (SSRI). In an embodiment, the SSRI is fluoxetine, paroxetine, sertraline, escitalopram or citalopram. In an embodiment, the second therapeutic agent is administered prior to, concurrently with or after R(−)-MDMA.

In an embodiment, the subject may also be undergoing psychotherapy treatment. In an embodiment, the psychotherapy treatment is Cognitive Processing Therapy (CPT), Cognitive Behavioral Therapy (CBT), Prolonged Exposure Therapy (PET), Brief Eclectic Psychotherapy (BEP), Narrative Exposure Therapy (NAT), or Eye-Movement Desensitization or Reprocessing (EMDR).

In one embodiment, the disclosure provides methods of activating 5-HT_{2A} and 5-HT_{2C} receptors in a subject including administering to the subject a therapeutically effective amount of a composition comprising R(−)-3,4-methylenedioxymethamphetamine (R(−)-MDMA) to lessen avoidance behavior in the subject.

In one embodiment, the disclosure provides methods of decreasing side effects of 3,4-methylenedioxy-methamphetamine (MDMA) treatment including administering to the subject a composition including R(−)-3,4-methylenedioxymethamphetamine (R(−)-MDMA) to lessen avoidance behavior in the subject.

In some embodiments, the methods of decreasing side effects that can be associated with MDMA treatment (i.e., a method comprising administering to a subject a composition comprising R(−)-MDMA) provide for improved methods of treatment and/or improved uses for various indications that are responsive to administration of MDMA. For example, in some embodiments, the disclosure provides improved methods for treating a subject who has a stress-related disease or disorder. In some further embodiments, the stress-related disease or disorder is post-traumatic stress disorder (PTSD). In such embodiments, the methods and uses can provide for a reduction or elimination of the occurrence, the frequency of occurrence, the duration of occurrence, and/or the severity of a side effect that is associated with administration of MDMA to a subject (i.e., administration of racemic and/or S(+)-MDMA). In some further embodiments the methods described herein, including the methods of decreasing side effects, comprise one or more of: a specific dosage of R(−)-MDMA, a specific delivery route of R(−)-MDMA, and/or a specific dosing schedule of R(−)-MDMA, such as provided by the disclosure and illustrative embodiments described herein.

In an embodiment, one or more side effects may be reduced (i.e., occurrence, frequency, duration, and/or severity of occurrence) and can include the non-limiting examples of cardiovascular effects, hyperthermia, neurotoxicity, or a combination thereof. In some example embodiments, the cardiovascular effects are increased blood pressure, increased heart rate, or a combination thereof. In some example embodiments, neurotoxicity includes mood disorder, cognition disorder and/or psychomotor deficits.

In an embodiment, R(−)-MDMA has antidepressant and anxiolytic effects.

In one embodiment, the disclosure provides methods for inducing neurite outgrowth in a subject including administering to the subject a therapeutically effective amount of a composition including R(−)-3,4-methylenedioxymethamphetamine (R(−)-MDMA). In embodiments, the method is effective to lessen avoidance behavior in the subject. In embodiments, neurite outgrowth includes increasing neurite number, neurite total length, number of neurite branch points per neuron or any combination thereof. In embodiments, the neurite outgrowth includes neurite outgrowth on prefrontal cortex neurons and/or hippocampal neurons.

In one embodiment, the disclosure provides methods of treating neuronal atrophy in a subject including administering to the subject a therapeutically effective amount of a composition including R(−)-3,4-methylenedioxymethamphetamine (R(−)-MDMA). In some embodiments, the method is effective to lessen avoidance behavior in the subject.

In one embodiment, the disclosure provides methods of inducing structural neuroplasticity in a subject including administering to the subject a therapeutically effective amount of a composition including R(−)-3,4-methylenedioxymethamphetamine (R(−)-MDMA). In some embodiments, the method is effective to lessen avoidance behavior in the subject.

In one embodiment, the disclosure provides methods of increasing brain-derived neurotrophic factor (BDNF) levels in a subject including administering to the subject a therapeutically effective amount of a composition including R(−)-3,4-methylenedioxymethamphetamine (R(−)-MDMA). In some embodiments, the method is effective to lessen avoidance behavior in the subject. In some embodiments, increasing BDNF levels includes increasing cerebral cortex and/or hippocampal BDNF levels. In some embodiments, increasing BDNF levels increases neuronal survival and/or synaptic plasticity.

In one embodiment, the disclosure provides pharmaceutical compositions including R(−)-MDMA and a pharmaceutically acceptable carrier.

In some further embodiments, the pharmaceutically acceptable carrier is saline or purified water.

### BRIEF DESCRIPTION OF THE DRAWINGS

FIG. 1A-1C shows the effect of MDMA on neurite growth. (FIG. 1A) racemic (R,S)-MDMA. (FIG. 1B) R(−)-MDMA. (FIG. 1C) S-MDMA. Veh is 0.1% sterile water; +Cnt1 is 0.25 uM Donepezil; * indicates significantly different from Veh (1-way ANOVA followed by Dunnett's test, p<0.05); # indicates significantly different from Veh (unpaired t test, p<0.05).

FIG. 2A-2C shows the effect of MDA on neurite growth. (FIG. 2A) racemic (R,S)-MDA. (FIG. 2B) R-MDA. (FIG. 2C) S-MDA. Veh is 0.1% sterile water; +Cnt1 is 0.25 uM Donepezil; # indicates significantly different from Veh (unpaired t test, p<0.05).

FIG. 3A-3C shows Head Twitch Response (HTR) after treatment with MDA. (FIG. 3A) R,S-MDA. (FIG. 3B) R-MDA. (FIG. 3C) S-MDA.

FIG. 4A-4C shows Head Twitch Response (HTR) after treatment with MDMA. (FIG. 4A) R,S-MDMA. (FIG. 4B) R(−)-MDMA. (FIG. 4C) S-MDMA.

FIG. 5 shows Extinction training and testing freezing (%) across days 3, 4, 8 and 13 of extinction training and testing sessions for all groups. Bars at each day (D) from left to right are vehicle; racemic MDMA; R-MDMA (10 mpk); R-MDMA (17 mpk); and R-MDMA (30 mpk).

FIG. 6 shows Extinction training and testing activity counts across days 3, 4, 8 and 13 of extinction training and testing sessions for all groups. Bars at each day (D) from left to right are vehicle; racemic MDMA; R-MDMA (10 mpk); R-MDMA (17 mpk); and R-MDMA (30 mpk).

FIG. 7 shows distance totals measured for 30 minutes over an 11 day period in mice injected on Day 1 with test vehicle or test article. Data shown are mean+/−SEM. Analysis was performed by two-way ANOVA with Fisher's LSD test *p<0.05, ***p<0.001 vs vehicle. Bars at each day (D) from left to right are vehicle; racemic MDMA; R-MDMA (10 mpk); R-MDMA (17 mpk); and R-MDMA (30 mpk).

FIG. 8 shows ambulatory counts measured for 30 minutes over an 11 day period in mice injected on Day 1 with test vehicle or test article. Data shown are mean+/−SEM. Analysis was performed by two-way ANOVA with Fisher's LSD test *p<0.05, ****p<0.0001 vs vehicle. Bars at each day (D) from left to right are vehicle; racemic MDMA; R-MDMA (10 mpk); R-MDMA (17 mpk); and R-MDMA (30 mpk).

FIG. 9 shows ambulatory time totals measured for 30 minutes over an 11 day period in mice injected on Day 1 with test vehicle or test article. Data shown are mean+/−SEM. Analysis was performed by two-way ANOVA with Fisher's LSD test *p<0.05, **p<0.01 vs vehicle, ****p<0.0001 vs vehicle. Bars at each day (D) from left to right are vehicle; racemic MDMA; R-MDMA (10 mpk); R-MDMA (17 mpk); and R-MDMA (30 mpk).

FIG. 10 shows vertical counts measured for 30 minutes over an 11 day period in mice injected on Day 1 with test vehicle or test article. Data shown are mean+/−SEM. Analysis was performed by two-way ANOVA with Fisher's LSD test ***p<0.001 vs vehicle, ****p<0.0001 vs vehicle. Bars at each day (D) from left to right are vehicle; racemic MDMA; R-MDMA (10 mpk); R-MDMA (17 mpk); and R-MDMA (30 mpk).

FIG. 11 shows vertical time totals measured for 30 minutes over an 11 day period in mice injected on Day 1 with test vehicle or test article. Data shown are mean+/−SEM. Analysis was performed by two-way ANOVA with Fisher's LSD test *p<0.05, **p<0.01 vs vehicle, ***p<0.001 vs vehicle, ****p<0.0001 vs vehicle. Bars at each day (D) from left to right are vehicle; racemic MDMA; R-MDMA (10 mpk); R-MDMA (17 mpk); and R-MDMA (30 mpk).

FIG. 12 depicts the heat map for behavioral changes as measured by FOB, with D labeled grid boxes indicating decrease in activity.

FIG. 13 illustrates the effects of enantiomeric-equivalent dosing conditions of R-MDMA, S-MDMA and racemic MDMA on heart rate change from baseline over time post initiation of dosing.

FIG. 14 illustrates the effects of enantiomeric-equivalent dosing conditions of R-MDMA, S-MDMA and racemic MDMA on systolic blood pressure change from baseline over time post initiation of dosing.

FIG. 15 illustrates the effects of enantiomeric-equivalent dosing conditions of R-MDMA, S-MDMA and racemic MDMA on diastolic blood pressure change from baseline over time post initiation of dosing.

FIG. 16 illustrates the effects of enantiomeric-equivalent dosing conditions of R-MDMA, S-MDMA and racemic MDMA on mean arterial blood pressure change from baseline over time post initiation of dosing.

FIG. 17 illustrates the effects of enantiomeric-equivalent dosing conditions of R-MDMA, S-MDMA and racemic MDMA on body temperature change from baseline over time post initiation of dosing.

FIG. 18 illustrates effects of enantiomeric-equivalent dosing conditions of R-MDMA, S-MDMA and racemic MDMA on activity change from baseline over time post initiation of dosing.

### DETAILED DESCRIPTION OF EMBODIMENTS

The disclosure exploits the different pharmacological and toxicological profiles, and thus the different functional characteristics possessed by R(−)-MDMA and S(+)-MDMA. Specifically, R(−)-MDMA induces neurite growth increasing neural plasticity and has many of the functional properties desirable for treating stress-related diseases or disorders such as PTSD.

The disclosure leverages the seminal discovery that R(−)-MDMA and S(+)-MDMA have different pharmacologic and toxicologic profiles and thus different functional characteristics. As detailed and described herein, it has surprisingly been determined that R(−)-MDMA induces neurite growth increasing neural plasticity and has many of the functional properties desirable for treating stress-related diseases or disorders such as PTSD and, can additionally provide for methods and uses that exhibit reduced negative and/or deleterious side effects relative to the side effects associated with racemic MDMA and/or (S)(+)-MDMA.

Before the various compositions and methods falling within the aspects and embodiments of the disclosure are described, it is to be understood that the description that follows is not limited to particular compositions, methods, and experimental conditions described; such compositions, methods, and conditions may vary without having a significant impact on efficacy or results. It is also to be understood that the terminology used herein is for purposes of describing particular aspects and embodiments only, and is not intended to be limiting to the scope of the appended claims.

As used in this specification and the appended claims, the singular forms “a”, “an”, and “the” include plural references unless the context clearly dictates otherwise. Thus, for example, references to “the method” includes one or more methods, and/or steps of the type described herein which will become apparent to those persons skilled in the art upon reading this disclosure and so forth.

All publications, patents, and patent applications mentioned in this specification are herein incorporated by reference to the same extent as if each individual publication, patent, or patent application was specifically and individually indicated to be incorporated by reference.

Unless defined otherwise, all technical and scientific terms used herein have the same meaning as commonly understood by a person skilled in the art to which this technology belongs. Although any methods and materials similar or equivalent to those described herein can be used in the practice or testing of the aspects and embodiments described herein, it will be understood that modifications and variations are encompassed within the spirit and scope of the disclosure. The various aspects and embodiments, inclusive of preferred methods and materials, are now described.

MDMA is a psychoactive compound that affects mood, perception, and increases prosocial feelings and behaviors. MDMA is a monoamine releaser and reuptake inhibitor and its prominent serotonergic effects differentiate it from amphetamine and methamphetamine. MDMA is considered the prototype entactogen compounds and produces subjective effects that are unlike any of the classical psychostimulants or hallucinogens and is one of the few compounds capable of reliably producing prosocial behavioral states.

Racemic MDMA enhances triple monoamine (serotonin, norepinephrine, and dopamine) release and inhibits reuptake, blockade of vesicular storage of monoamines, inhibition of neurotransmitter oxidation by MAO-A, and a reversal of 5-HT transport into the neuron. These effects result in a net increase of monoamines in the synaptic cleft and prolong the duration of monoaminergic neurotransmission.

MDMA produces anxiolytic and prosocial effects including increased compassion for self and others, reduced defenses and fear of emotional injury, and making unpleasant memories less disturbing while enhancing communication and capacity for introspection. Collectively, these factors provide the opportunity for a corrective emotional experience in the context of therapy. However, adverse events have been observed in clinical and nonclinical studies of MDMA, such as cardiovascular effects, hyperthermia and neurotoxicity that may limit its clinical viability.

As the molecular structure of MDMA has a chiral center, there are two stereoisomers S(+)-MDMA and R(−)-MDMA. The enantiomers have been shown to impact the monoaminergic targets of MDMA differently, resulting in pharmacological activity of S(+)-MDMA resembling those of psychostimulants, including increases in motor activity and euphoria, and R(−)-MDMA activity inducing effects closer to classical psychedelics, such as alteration in perception and ego-dissolution. Indeed, the differing pharmacological and toxicological profile indicate that R(−)-MDMA can provide an improved therapeutic index, offering the therapeutic effects of racemic MDMA but with a reduced side effect profile.

For purposes of clarity, as used herein reference to, or usage of, the term “MDMA” typically refers to the racemic form of MDMA (i.e., essentially equal amounts of both stereoisomers S(+)-MDMA and R(−)-MDMA), while reference to an individual MDMA enantiomer will typically identify or indicate one or the other isomer in its optically or enantiomerically pure form, or substantially pure form (i.e., S(+)-MDMA, S-MDMA, R-MDMA, or R(−)-MDMA). In some occurrences within the disclosure and Figures, a formulation can include R(−)-MDMA in a large excess relative to the S(+)-MDMA isomer (e.g., at least 90% R(−)-MDMA or more), and in some embodiments a composition can comprise an optically or enantiomerically pure form, or substantially pure form, of R(−)-MDMA, allowing for amounts of the S(+)-MDMA enantiomer that can be considered as insignificant and/or an impurity.

### Methods of Treating Stress-Related Diseases and Disorders

In one embodiment, the disclosure provides methods of treating a stress-related disease or disorder in a subject including administering to the subject a therapeutically effective amount of a composition including R(−)-3,4-methylenedioxymethamphetamine (R(−)-MDMA) to lessen avoidance behavior in the subject.

Enantiomers are pairs of compounds with exactly the same connectivity but opposite three-dimensional shapes. Enantiomers are stereoisomers, i.e. mirror images of each other. Enantiomers can have different chemical and biologic properties. R(−)-MDMA and S(+)-MDMA are the enantiomers of MDMA. R(−)-MDMA and S(+)-MDMA have different toxicologic and pharmacologic properties. For example, R(−)-MDMA, but not S(+)-MDMA, binds to 5-HT_{2A} and 5-HT_{2C} receptors.

The composition includes for example, at least 90% R(−)-MDMA and 10% S(+)-MDMA; at least 91% R(−)-MDMA and 9% S(+)-MDMA; at least 92% R(−)-MDMA and 8% S(+)-MDMA; at least 93% R(−)-MDMA and 7% S(+)-MDMA; at least 94% R(−)-MDMA and 6% S(+)-MDMA; at least 95% R(−)-MDMA and 5% S(+)-MDMA; at least 96% R(−)-MDMA and 4% S(+)-MDMA; at least 97% R(−)-MDMA and 3% S(+)-MDMA; at least 98% R(−)-MDMA and 2% S(+)-MDMA; at least 99% R(−)-MDMA and 1% S(+)-MDMA; or 100% R(−)-MDMA and 0% S(+)-MDMA. In an embodiment, the compositions includes about 99.1% R(−)-MDMA and 0.9% S(+)-MDMA; about 99.2% R(−)-MDMA and 0.8% S(+)-MDMA; about 99.3% R(−)-MDMA and 0.7% S(+)-MDMA; about 99.4% R(−)-MDMA and 0.6% S(+)-MDMA; about 99.5% R(−)-MDMA and 0.5% S(+)-MDMA; about 99.6% R(−)-MDMA and 0.4% S(+)-MDMA; about 99.7% R(−)-MDMA and 0.3% S(+)-MDMA; about 99.8% R(−)-MDMA and 0.2% S(+)-MDMA; or about 99.9% R(−)-MDMA and 0.1% S(+)-MDMA.

In one embodiment, the composition includes 90-100% R(−)-MDMA and 0-10% S(+)-MDMA. In one embodiment, the composition includes 91-100% R(−)-MDMA and 0-9% S(+)-MDMA. In one embodiment, the composition includes 92-100% R(−)-MDMA and 0-8% S(+)-MDMA. In one embodiment, the composition includes 93-100% R(−)-MDMA and 0-7% S(+)-MDMA. In one embodiment, the composition includes 94-100% R(−)-MDMA and 0-6% S(+)-MDMA. In one embodiment, the composition includes 95-100% R(−)-MDMA and 0-5% S(+)-MDMA. In one embodiment, the composition includes 96-100% R(−)-MDMA and 0-4% S(+)-MDMA. In one embodiment, the composition includes 97-100% R(−)-MDMA and 0-3% S(+)-MDMA. In one embodiment, the composition includes 98-100% R(−)-MDMA and 0-2% S(+)-MDMA. In one embodiment, the composition includes 99-100% R(−)-MDMA and 0-1% S(+)-MDMA.

The term “subject” as used herein refers to any individual or patient to which the subject methods are performed. Generally, the subject is human, although as will be appreciated by those in the art, the subject may be an animal. Thus, other animals, including vertebrate such as rodents (including mice, rats, hamsters and guinea pigs), cats, dogs, rabbits, farm animals including cows, horses, goats, sheep, pigs, chickens, etc., and primates (including monkeys, chimpanzees, orangutans and gorillas) are included within the definition of subject.

The terms “treat,” “treated,” “treating”, or “treatment” as used herein refers to both therapeutic treatment and prophylactic or preventative measures, wherein the object is to prevent or slow down (lessen) an undesired physiological condition, disorder or disease, or to obtain beneficial or desired clinical results. For the purposes of this description, beneficial or desired clinical results include, but are not limited to, alleviation of symptoms; diminishment of the extent of the condition, disorder or disease; stabilization (i.e., not worsening) of the state of the condition, disorder or disease; delay in onset or slowing of the progression of the condition, disorder or disease; amelioration of the condition, disorder or disease state; and remission (whether partial or total, whether induction of or maintenance of), whether detectable or undetectable, or enhancement or improvement of the condition, disorder or disease. Treatment includes eliciting a clinically significant response without excessive levels of side effects. Treatment also includes prolonging survival as compared to expected survival if not receiving treatment. Treatment may also be preemptive in nature, i.e., it may include prevention of disease. Prevention of a disease may involve complete protection from disease, for example as in the case of prevention of infection with a pathogen or may involve prevention of disease progression. For example, prevention of a disease may not mean complete foreclosure of any effect related to the diseases at any level, but instead may mean prevention of the symptoms of a disease to a clinically significant or detectable level. Prevention of diseases may also mean prevention of progression of a disease to a later stage of the disease and prolonging disease-free survival as compared to disease-free survival if not receiving treatment and prolonging disease-free survival as compared to disease-free survival if not receiving treatment.

The term “treatment” is used interchangeably herein with the term “therapeutic method” and refers to both 1) therapeutic treatments or measures that cure, slow down, lessen symptoms of, and/or halt progression of a diagnosed pathologic conditions or disorder, and 2) prophylactic/preventative measures. Those in need of treatment may include individuals already having a particular medical disorder as well as those who may ultimately acquire the disorder (i.e., those needing preventive measures).

The terms “therapeutically effective amount”, “effective dose,” “therapeutically effective dose”, “effective amount,” or the like refer to that amount of the subject compound that will elicit the biological or medical response of a tissue, system, animal or human that is being sought by the researcher, veterinarian, medical doctor or other clinician. Generally, the response is either amelioration of symptoms in a patient or a desired biological outcome (e.g., treatment of a stress-related disease or disorder). The effective amount can be determined as described herein.

The compositions of the disclosure include an amount of a composition including R(−)-MDMA, wherein such composition is effective for treating or alleviating the symptoms of stress-related disease or disorder in a subject. Specifically, the dosage of the composition to achieve a therapeutic effect will depend on factors such as the formulation, pharmacological potency of the composition, age, weight and sex of the patient, condition being treated, severity of the patient's symptoms, route of delivery, and response pattern of the patient. It is also contemplated that the treatment and dosage of the compositions may be administered in unit dosage form and that one skilled in the art would adjust the unit dosage form accordingly to reflect the relative level of activity. The decision as to the particular dosage to be employed (and the number of times to be administered per day) is within the competency and discretion of a skilled physician and may be varied by titration of the dosage to the particular circumstances to produce the therapeutic effect. Further, one of skill in the art would be able to calculate any changes in effective amounts of the compositions due to changes in the composition components or dilutions. In one aspect, the compositions may be diluted 2-fold. In another aspect, the compositions may be diluted 4-fold. In a further aspect, the compositions may be diluted 8-fold.

The effective amount of the compositions disclosed herein may, therefore, be about 1 mg to about 1000 mg per dose based on a 60 kg mammalian, for example human, subject. In an embodiment, the therapeutically effective amount is about 1 mg to about 750 mg per dose. In an embodiment, the therapeutically effective amount is about 5 mg to about 500 mg, the therapeutically effective amount is about 10 mg to about 400 mg, the therapeutically effective amount is about 25 mg to about 300 mg, the therapeutically effective amount is about 75 mg to about 225 mg, the therapeutically effective amount is about 75 mg to about 850 mg, the therapeutically effective amount is about 250 mg to about 850 mg, the therapeutically effective amount is about 250 mg to about 350 mg. In an embodiment, the therapeutically effective amount is about 25 mg to 50 mg, about 20 mg, about 15 mg, about 10 mg, about 5 mg, about 1 mg, about 0.1 mg, about 0.01 mg, about 0.001 mg. The therapeutically effective amount is about 25 mg, 26 mg, 27 mg, 28 mg, 29 mg, 30 mg, 31 mg, 32 mg, 33 mg, 34 mg, 35 mg, 36 mg, 37 mg, 38 mg, 39 mg, 40 mg, 41 mg, 42 mg, 43 mg, 44 mg, 45 mg, 46 mg, 47 mg, 48 mg, 49 mg, 50 mg, 51 mg, 52 mg, 53 mg, 54 mg, 55 mg, 56 mg, 57 mg, 58 mg, 59 mg, 60 mg, 61 mg, 62 mg, 63 mg, 64 mg, 65 mg, 66 mg, 67 mg, 68 mg, 69 mg, 70 mg, 71 mg, 72 mg, 73 mg, 74 mg, 75 mg, 76 mg, 77 mg, 78 mg, 79 mg, 80 mg, 81 mg, 82 mg, 83 mg, 84 mg, 85 mg, 86 mg, 87 mg, 88 mg, 89 mg, 90 mg, 91 mg, 92 mg, 93 mg, 94 mg, 95 mg, 96 mg, 97 mg, 98 mg, 99 mg, 100 mg, 101 mg, 102 mg, 103 mg, 104 mg, 105 mg, 106 mg, 107 mg, 108 mg, 109 mg, 110 mg, 111 mg, 112 mg, 113 mg, 114 mg, 115 mg, 116 mg, 117 mg, 118 mg, 119 mg, 120 mg, 121 mg, 122 mg, 123 mg, 124 mg, 125 mg, 126 mg, 127 mg, 128 mg, 129 mg, 130 mg, 131 mg, 132 mg, 133 mg, 134 mg, 135 mg, 136 mg, 137 mg, 138 mg, 139 mg, 140 mg, 141 mg, 142 mg, 143 mg, 144 mg, 145 mg, 146 mg, 147 mg, 148 mg, 149 mg, 150 mg, 151 mg, 152 mg, 153 mg, 154 mg, 155 mg, 156 mg, 157 mg, 158 mg, 159 mg, 160 mg, 161 mg, 162 mg, 163 mg, 164 mg, 165 mg, 166 mg, 167 mg, 168 mg, 169 mg, 170 mg, 171 mg, 172 mg, 173 mg, 174 mg, 175 mg, 176 mg, 177 mg, 178 mg, 179 mg, 180 mg, 181 mg, 182 mg, 183 mg, 184 mg, 185 mg, 186 mg, 187 mg, 188 mg, 189 mg, 190 mg, 191 mg, 192 mg, 193 mg, 194 mg, 195 mg, 196 mg, 197 mg, 198 mg, 199 mg, 200 mg, 201 mg, 202 mg, 203 mg, 204 mg, 205 mg, 206 mg, 207 mg, 208 mg, 209 mg, 210 mg, 211 mg, 212 mg, 213 mg, 214 mg, 215 mg, 216 mg, 217 mg, 218 mg, 219 mg, 220 mg, 221 mg, 222 mg, 223 mg, 224 mg, 225 mg, 226 mg, 227 mg, 228 mg, 229 mg, 230 mg, 231 mg, 232 mg, 233 mg, 234 mg, 235 mg, 236 mg, 237 mg, 238 mg, 239 mg, 240 mg, 241 mg, 242 mg, 243 mg, 244 mg, 245 mg, 246 mg, 247 mg, 248 mg, 249 mg, 250 mg, 251 mg, 252 mg, 253 mg, 254 mg, 255 mg, 256 mg, 257 mg, 258 mg, 259 mg, 260 mg, 261 mg, 262 mg, 263 mg, 264 mg, 265 mg, 266 mg, 267 mg, 268 mg, 269 mg, 270 mg, 271 mg, 272 mg, 273 mg, 274 mg, 275 mg, 276 mg, 277 mg, 278 mg, 279 mg, 280 mg, 281 mg, 282 mg, 283 mg, 284 mg, 285 mg, 286 mg, 287 mg, 288 mg, 289 mg, 290 mg, 291 mg, 292 mg, 293 mg, 294 mg, 295 mg, 296 mg, 297 mg, 298 mg, 299 mg, 300 mg, or more.

The effective dose of the compositions disclosed herein may, therefore, be about 0.01 mg/kg to about 20 mg/kg per dose. In an embodiment, the therapeutically effective dose is about 1 mg/kg to about 15 mg/kg per dose. In an embodiment, the therapeutically effective amount is about 5 mg/kg to about 15 mg/kg, the therapeutically effective amount is about 10 mg/k to about 20 mg/kg, the therapeutically effective amount is about 3 mg/kg to about 15 mg/kg, the therapeutically effective amount is about 3 mg/kg to about 5 mg/kg. In an embodiment, the therapeutically effective amount is about 0.1 mg/kg to 50 mg/kg, about 0.001 mg/kg, about 0.01 mg/kg, about 0.1 mg/kg, about 1 mg/kg, about 5 mg/kg, about 10 mg/kg, about 25 mg/kg, about 30 mg/kg, about 40 mg/kg. In an embodiment, the therapeutically effective amount is about 0.1 mg/kg, 0.25 mg/kg, 0.5 mg/kg, 0.75 mg/kg, 1.0 mg/kg, 1.25 mg/kg, 1.5 mg/kg, 1.75 mg/kg, 2.0 mg/mg, 2.25 mg/kg, 2.5 mg/kg, 2.75 mg/kg, 3.0 mg/kg, 3.25 mg/kg, 3.5 mg/kg, 3.75 mg/kg, 4.0 mg/kg, 4.25 mg/kg, 4.5 mg/kg, 4.75 mg/kg, 5.0 mg/kg, 5.25 mg/kg, 5.5 mg/kg, 5.75 mg/kg, 6.0 mg/kg, 6.25 mg/kg, 6.5 mg/kg, 6.75 mg/kg, 7.0 mg/kg, 7.25 mg/kg, 7.5 mg/kg, 7.75 mg/kg, 8.0 mg/kg, 8.5 mg/kg, 9.0 mg/kg, 9.5 mg/kg, 10.0 mg/kg, 10.5 mg/kg, 11.0 mg/kg, 11.5 mg/kg, 12.0 mg/kg, 12.5 mg/kg, 13.0 mg/kg, 13.5 mg/kg, 14.0 mg/kg, 15.5 mg/kg, 16.0 mg/kg, 16.5 mg/kg, 17.0 mg/kg, 17.5 mg/kg, 18.0 mg/kg, 18.5 mg/kg, 19.0 mg/kg, 19.5 mg/kg, 20.0 mg/kg, 20.5 mg/kg, 21 mg/kg, 22, mg/kg, 23, mg/kg, 24 mg/kg, 25 mg/kg, 26 mg/kg, 27 mg/kg, 28 mg/kg, 29 mg/kg, 30 mg/kg, 31 mg/kg, 32, mg/kg, 33, mg/kg, 34 mg/kg, 35 mg/kg, 36 mg/kg, 37 mg/kg, 38 mg/kg, 39 mg/kg, 40 mg/kg, 41 mg/kg, 42, mg/kg, 43, mg/kg, 44 mg/kg, 45 mg/kg, 46 mg/kg, 47 mg/kg, 48 mg/kg, 49 mg/kg, and 50 mg/kg. The effective amounts may be provided as a single dose or on regular schedule, i.e., on a daily, weekly, monthly, or yearly basis or on an irregular schedule with varying administration days, weeks, months, etc. To reduce the occurrence of possible side effect associated with the dose, an effective amount may be provided as a split dose, where the single dose is split into two doses, that are administered apart, usually over several hours. For example, a single dose may be split into two doses, administered 1 hour apart, 2 hours apart, 3 hours apart, 4 hours apart, 5 hours apart, 6 hours apart, 7 hours apart, 8 hours apart, or more. Alternatively, the therapeutically effective amount to be administered may vary. In one aspect, the therapeutically effective amount for the first dose is higher than the therapeutically effective amount for one or more of the subsequent doses. In another aspect, the therapeutically effective amount for the first dose is lower than the therapeutically effective amount for one or more of the subsequent doses. Equivalent dosages may be administered over various time periods including, but not limited to, about every 2 hours, about every 6 hours, about every 8 hours, about every 12 hours, about every 24 hours, about every 36 hours, about every 48 hours, about every 72 hours, about every week, about every 2 weeks, about every 3 weeks, about every month, about every 2 months, about every 3 months and about every 6 months. The number and frequency of dosages corresponding to a completed course of therapy will be determined according to the judgment of a health-care practitioner.

The terms “administration of” and or “administering” should be understood to mean providing a pharmaceutical composition in a therapeutically effective amount to the subject in need of treatment. The compositions may be administered by any route, taking into consideration the specific condition for which it has been selected. The compositions may be delivered orally, by injection, inhalation (including orally, intranasally and intratracheally), ocularly, transdermally (via simple passive diffusion formulations or via facilitated delivery using, for example, iontophoresis, microporation with microneedles, radio-frequency ablation or the like), intravascularly, cutaneously, subcutaneously, intramuscularly, sublingually, intracranially, epidurally, rectally, intravesically, and vaginally, among others.

In an embodiment, the compositions of the disclosure are administered by intracutaneous, subcutaneous, intravenous, intraarterial, intradermal, transdermal, oral, sublingual buccal, or nasal route of administration.

Although the compositions may be administered alone, they may also be administered in the presence of one or more pharmaceutical carriers that are physiologically compatible. The carriers may be in dry or liquid form and must be pharmaceutically acceptable. Liquid pharmaceutical compositions may be sterile solutions or suspensions. When liquid carriers are utilized, they may be sterile liquids. Liquid carriers may be utilized in preparing solutions, suspensions, emulsions, syrups and elixirs. In one aspect, the compositions may be dissolved a liquid carrier. In another aspect, the compositions may be suspended in a liquid carrier. One of skill in the art of formulations would be able to select a suitable liquid carrier, depending on the route of administration. The compositions may alternatively be formulated in a solid carrier. In one aspect, the composition may be compacted into a unit dose form, i.e., tablet or caplet. In another aspect, the composition may be added to unit dose form, i.e., a capsule. In a further aspect, the composition may be formulated for administration as a powder. The solid carrier may perform a variety of functions, i.e., may perform the functions of two or more of the excipients described below. For example, a solid carrier may also act as a flavoring agent, lubricant, solubilizer, suspending agent, filler, glidant, compression aid, binder, disintegrant, or encapsulating material. In one aspect, a solid carrier acts as a lubricant, solubilizer, suspending agent, binder, disintegrant, or encapsulating material. The composition may also be sub-divided to contain appropriate quantities of the compositions. For example, the unit dosage can be packaged compositions, e.g., packeted powders, vials, ampoules, prefilled syringes or sachets containing liquids

Where the compositions include a pharmaceutical carrier(s), the amount of the pharmaceutical carrier(s) is determined by the solubility and chemical nature of the peptides, chosen route of administration and standard pharmacological practice. The pharmaceutical carrier(s) may be solid or liquid and may incorporate both solid and liquid carriers/matrices. A variety of suitable liquid carriers is known and may be readily selected by one of skill in the art. Such carriers may include, e.g., dimethylsulfoxide (DMSO), saline, buffered saline, purified water, cyclodextrin, hydroxypropylcyclodextrin (HPβCD), n-dodecyl-β-D-maltoside (DDM) and mixtures thereof. Similarly, a variety of solid (rigid or flexible) carriers and excipients are known to those of skill in the art.

In an embodiment, the subject has a stress-related disease or disorder. In embodiments, the methods comprise treating stress-related disease or disorder including, for example, post-traumatic stress disorder, social anxiety, autism spectrum disorder, substance use disorder, depression, anxiety disorder, anxiety with life-threatening disease, personality disorder including narcistic or antisocial personality disorder, schizophrenia, obsessive compulsive disorder, gambling, aberrant sexual behavior, an auditory disorder, an additive disorder (e.g., substance use disorder such as alcohol abuse, substance abuse, smoking), an eating disorder (anorexia nervosa, bulimia nervosa, binge eating disorder, etc.), an auditory disorder, an impulsive disorder (e.g., attention deficit hyperactivity disorder (ADHD), attention deficit disorder (ADD), or Tourette's syndrome), enhancing of a psychotherapy and/or enhancing or inducing feelings of well-being connectivity, trust, love, empathy, openness, and pro-sociality.

In an embodiment, the stress-related disease or disorder is mood/depressive disorder, bipolar disorder, anxiety disorder, psychotic or delirium disorder, schizophrenia, schizoaffective disorder, personality disorder, abuse or neglect disorder, tic disorder, neurocognitive disorder, neurodevelopmental disorder, learning disorder, disruptive mood regulation disorder, intermittent explosive disorder, antisocial personality disorder, conduct disorder, behavioral and psychological symptoms of dementia, depression, treatment resistant depression, anxiety, post-traumatic stress disorder (PTSD), or any combination. In an embodiment, the stress-related disease or disorder is PTSD, or any combination thereof.

PTSD is a serious, chronic, life-threatening psychiatric disorder. Psychiatric symptoms of PTSD are debilitating and occur after experiencing a single traumatic event or repeated traumatic experiences, such as violence, accidents, sexual and/or childhood abuse, natural disasters, terrorism, and war. Symptoms include recurring and intrusive negative thoughts or recollections of the traumatic event, cognitive disruption, hyperarousal to event related cues, and avoidance behaviors that persist for longer periods than a month after experiencing a traumatic event. Overall reduction in the quality of life is common in individuals with PTSD leading to disability and can affect physical health with manifestation of other comorbidities such as cardiovascular disease, concomitant mental health conditions and suicidality.

Two serotonin reuptake inhibitors (SSRIs) are currently approved for the treatment of PTSD, sertraline (Zoloft®) and paroxetine (Paxil®), which, like racemic MDMA, increase the level of serotonin in the synaptic cleft. Sertraline and paroxetine have demonstrated moderate efficacy in reducing PTSD symptoms, but rarely result in full disorder remission. These pharmacotherapies also have problematic side effects and generally require long-term and/or consistent use to maintain effectiveness, although long-term compliance is poor. Studies have demonstrated that administering stand-alone sertraline or paroxetine reported significantly decreased reduction in PTSD symptoms compared to currently available behavioral interventions.

Based on the low response rate to existing pharmacotherapy, the most recent clinical practice guidelines recommend psychotherapy as the first-line treatment for PTSD. Specifically, the American Psychological Association (APA) and US Departments of Defense and Departments of Veterans Affairs (DoD/VA) practice guidelines recommend Cognitive Processing Therapy (CPT), Cognitive Behavioral Therapy (CBT), Prolonged Exposure Therapy (PET), Brief Eclectic Psychotherapy (BEP), Narrative Exposure Therapy (NAT), and Eye-Movement Desensitization and Reprocessing (EMDR), as first-line treatment for PTSD as these treatments have repeatedly demonstrated efficacy in reducing symptoms of PTSD in randomized clinical trials. However, it has been shown that CPT and PE therapy did not lead to remission or even clinically meaningful reductions in symptoms in the majority of patients with PTSD. This is consistent with several studies that estimate that between 40%-60% of patients receiving any treatment for PTSD do not respond adequately and/or continue to meet diagnostic criteria after receiving treatment.

One of the symptoms of PTSD is learned avoidance behavior. Avoidance is a safety-seeking or protective response in response to trauma. However, as this avoidance behavior becomes more extreme, a person's quality of life may lessen. One type of behavioral therapy for PTSD is Exposure Therapy which can reduce anxiety and ultimately eliminating avoidance behavior and improving the quality of life for a subject having PTSD. As shown in the examples below, R(−)-MDMA is effective for reducing anxiety and avoidance behavior as shown in an animal model of fear extinction. In the animal model, freezing was significantly reduced on the days of and following single dosing of R-MDMA.

Depressive disorders are characterized by low mood, feeling sad or hopeless, increased irritability, sleep disturbance, lowered energy and feeling tired, poor concentration, lowered self-esteem or feeling worthless, lack of interest or pleasure in things, slowed movement, thinking they would be better off dead and self-harm, and actively considering and attempting suicide, and include major depressive disorder, bipolar depression, treatment resistant depression, and dysthymic disorder.

Anxiety disorders are characterized by feeling nervous, anxious, having difficulty breathing, avoiding people or activities, worrying excessively, physical symptoms including chest pain, palpitations of the heart, sweating, nausea, stomachache, headache, neckache, poor sleep, poor concentration, sweating, and dizziness and include generalized anxiety disorder, social anxiety disorder, panic disorder, agoraphobia, phobias, and separation anxiety disorder.

Because prosocial effects of R-MDMA may facilitate therapeutic engagement and efficacy, any conditions that can be treated using psychotherapy, including but not limited to mood/depressive disorders, bipolar disorders, anxiety disorders, psychotic or delirium disorders, schizophrenia or schizoaffective disorders, personality disorders, abuse or neglect disorders, tic disorders, neurocognitive disorders, neurodevelopmental disorders, learning disorders, etc. may benefit from a treatment with R-MDMA. Conditions accompanied by increased aggression and/or irritability (e.g., disruptive mood regulation disorder, intermittent explosive disorder, antisocial personality disorder, conduct disorder, other personality disorders, other neurodevelopmental disorders, behavioral and psychological symptoms of dementia, etc.) may also benefit from the effects of R-MDMA.

In an embodiment, R(−)-MDMA has an antidepressant and anxiolytic effect.

Serotonin receptors (i.e., 5-HT receptors), are a group of G protein-coupled receptors and ligand-gated ion channels found in the central and peripheral nervous systems. The receptors mediate both excitatory and inhibitory neurotransmission. The 5-HT receptors modulate the release of many neurotransmitters, including glutamate, GABA, dopamine, epinephrine/norepinephrine, and acetylcholine, as well as many hormones, including oxytocin, prolactin, vasopressin, cortisol, corticotropin, and substance P, among others. The 5-HT receptors influence various biological and neurological processes such as aggression, anxiety, appetite, cognition, learning, memory, mood, nausea, sleep, and thermoregulation. They are the target of a variety of pharmaceutical and recreational drugs, including many antidepressants, antipsychotics, anorectics, antiemetics, gastroprokinetic agents, antimigraine agents, hallucinogens, and entactogens. There are a variety of 5-HT receptors (e.g., 5-HT₁, 5-HT₂, 5-HT₃, 5-HT₄, 5-HT₅, 5-HT₆, 5-HT 7) which have differing functions. There are also 5-HT receptor subtypes (e.g., 5-HT_{1A}, 5-HT_{1B}, 5-HT_{1D}, 5-HT_{1E}, 5-HT_{1F}, 5-HT_{2A}, 5-HT_{2B}, 5-HT_{2C}, 5-HT_{5A}, 5-HT_{5B}). For example, 5-HT_{2A} is involved in addiction, anxiety, appetite, cognition, imagination, learning, memory, mood, perception, sexual behavior, sleep, thermoregulation and vasoconstriction. In another example, 5-HT_{2C} is involved in addiction, anxiety, appetite, GI motility, heteroreceptor for norepinephrine and dopamine, locomotion, mood, sexual behavior, sleep, thermoregulation and vasoconstriction.

The compositions comprising R(−)-MDMA can be administered alone or in combination with one or more additional therapeutic agents. The phrases “combination therapy”, “combined with” and the like refer to the use of more than one medication or treatment simultaneously to increase the response. The composition in accordance with example aspects and embodiments of the disclosure may, for example, be used in combination with other drugs or treatment in use to stress-related diseases or disorders. In one specific embodiment, the administration of R(−)-MDMA to a subject can be in combination with a selective serotonin reuptake inhibitor (SSRI) or an alpha 7 nicotinic acetylcholine receptor (α7 nAChR) modulator.

Selective serotonin reuptake inhibitors (SSRIs) are a class of drugs that are typically used as antidepressants in the treatment of major depressive disorder, anxiety disorders, and other psychological conditions. SSRIs increase the extracellular level of the neurotransmitter serotonin by limiting its reabsorption (reuptake) into the presynaptic cell. These agents have varying degrees of selectivity for the other monoamine transporters, with pure SSRIs having strong affinity for the serotonin transporter and only weak affinity for the norepinephrine and dopamine transporters. SSRIs include fluoxetine, paroxetine, sertraline, escitalopram and citalopram.

In an embodiment, the R(−)-MDMA composition is administered with a therapeutic agent. In an embodiment, the therapeutic agent is a SSRI. In an embodiment the therapeutic agent is fluoxetine, paroxetine, sertraline, and/or escitalopram and citalopram. In an embodiment, such therapies can be administered prior to, simultaneously with, or following administration of the R(−)-MDMA composition.

The R(−)-MDMA compositions in accordance with the disclosure may be administered to subjects undergoing psychotherapy. In an embodiment, the psychotherapy treatment is Cognitive Processing Therapy (CPT), Cognitive Behavioral Therapy (CBT), Prolonged Exposure Therapy (PET), Brief Eclectic Psychotherapy (BEP), Narrative Exposure Therapy (NAT) or Eye-Movement Desensitization and Reprocessing (EMDR).

In one embodiment, there is provided a composition comprising or consisting of R(−)-MDMA as the active agent, for use in treating post-traumatic stress disorder. In one embodiment, there is provided a method of treating a stress-related disease or disorder by administering a composition comprising or consisting of R(−)-MDMA as the active agent.

Method of Activating 5-HT_{2A} and 5-HT_{2C} Receptors

In one embodiment, the disclosure provides a method of activating 5-HT_{2A} and/or 5-HT_{2C} receptors in a subject including administering to the subject a composition including an effective amount of R(−)-3,4-methylenedioxymethamphetamine (R(−)-MDMA) to activate the 5-HT_{2A} and/or 5-HT_{2C} receptors, and lessen avoidance behavior in the subject.

In an embodiment, R(−)-MDMA activates the 5-HT_{2A} and 5-HT_{2C} receptors. In an embodiment, R(−)-MDMA is a partial agonist of 5-HT_{2A}. In some further embodiments, activation or at least partial activation of the 5-HT_{2C} receptor can induce the therapeutic effects of R(−)-MDMA, including, for example, the reduction in side effects that are associated with racemic- and/or S(+)-MDMA.

### Reduction of MDMA Related Side Effects

The initial therapeutic potential of MDMA for trauma-related psychopathology developed as a potential catalyst for psychotherapeutic processes by facilitating communication and connection between patient and therapist. Clinical trials of MDMA are generally well tolerated with low potential for abuse. Adverse effects were dose dependent that included anxiety, dizziness, jaw clenching/tight jaw, lack of appetite and nausea. Acute hyperthermia also presents a concern, given that even at modest levels may lead to death. Chronic use of MDMA can also lead to neurotoxicity, but it is unclear whether low or moderate lifetime usage is affected or if neurotoxicity is a risk from clinical use of MDMA. To mitigate these potential adverse effects, and as demonstrated by the illustrative examples described in detail below, the individual enantiomers of racemic MDMA, and in particular embodiments, the R(−)-MDMA enantiomer can improve the drug's therapeutic index while reducing the side effect profile. In embodiments illustrated herein, various dosage forms, dosages, and dosing schedules can impact both the efficacy of the methods and the degree to which the side effect profile is reduced.

R(−)-MDMA is a partial 5-HT_{2A} receptor agonist, directly activating post-synaptic 5-HT_{2A} receptors, while S(+)-MDMA has no agonist activity at 5-HT_{2A} receptors. As the 5-HT_{2A} receptor has been implicated in the psychedelic effects of drugs, the psychedelic-like effects of racemic MDMA are likely associated with R(−)-MDMA activity. R(−)-MDMA is the more potent 5-HT_{2A} receptor agonist, S(+)-MDMA is a more potent inhibitor of serotonin transporter (SERT). Studies have shown that S(+)-MDMA has greater dopaminergic and noradrenergic activity than R(−)-MDMA. Given that S(+)-MDMA appears to be the primary driver for the release of dopamine and norepinephrine, it is likely responsible for much of the sympathomimetic side effects associated with administration of racemic MDMA. These sympathomimetic effects of racemic MDMA include dose-dependent transient increases in heart rate and blood pressure. 3,4-methylenedioxyamphetamine (MDA) is one of the major MDMA metabolites and has two stereoisomers, R(−)-MDA and S(+)-MDA. It is thought that these metabolites may have functional properties similar to MDMA, R(−)-MDMA and/or S(+)-MDMA. Identification of specific enantiomers of MDMA or MDA that do not induce and/or increase hyperthermia and other sympathomimetic effects, but still promote prosocial behaviors and extinction of fear driven responses, such as the R(−)-MDMA enantiomer as demonstrated in the Examples below, is particularly useful in the treatment of PTSD and other stress-related diseases and disorders.

In one embodiment, the present invention provides methods of decreasing side effects of 3,4-methylenedioxy-methamphetamine (MDMA) treatment including administering to the subject a therapeutically effective amount of a composition including, comprising, and/or consisting of R(−)-3,4-methylenedioxymethamphetamine (R(−)-MDMA) to lessen avoidance behavior in the subject.

In an embodiment, the side effects include cardiovascular effects, hyperthermia and/or neurotoxicity. In an embodiment, the cardiovascular effects are increased blood pressure, increased heart rate or any combination thereof. In an embodiment, neurotoxicity includes mood disorder, cognition disorder, psychomotor deficits and combinations thereof.

In an embodiment, the subject has a stress-related disease or disorder including depression, anxiety, post-traumatic stress disorder (PTSD). In an embodiment, the stress-related disease or disorder is PTSD.

In an embodiment, R(−)-MDMA has antidepressant and anxiolytic effects. In an embodiment, R(−)-MDMA activates the 5-HT_{2A} and 5-HT_{2C} receptors and is a partial agonist of 5-HT_{2A}. In an embodiment, R(−)-MDMA induces neurite growth. In an embodiment, administering includes administering between about 1 mg/kg to 20 mg/kg R(−)-MDMA. In an embodiment, the administering includes intracutaneous, subcutaneous, intravenous, intraarterial, intradermal, transdermal, oral, sublingual, buccal, or nasal route of administration.

### Neurite Outgrowth

Signs of neuronal atrophy have been found in brain regions involved in stress-related behaviors, including prefrontal cortex and hippocampus. In animals, these structural changes have been shown to include loss of neurites, dendritic spines and synaptic contacts, as well as reduced hippocampal neurogenesis. It has been shown that chronic, but not acute, administration of typical antidepressant drugs, such as selective serotonin reuptake inhibitors (SSRIs), attenuate the effects of stress on neurogenesis and neuronal structure in animals and demonstrate antidepressant and anxiolytic effects in humans. Thus, compounds that promote the generation and maintenance of neurites, spines, synapses and/or neurons upon single administration may have rapid therapeutic benefit in the treatment of stress-related disorders. Neurite outgrowth is measured by increasing neurite number, neurite total length and total number of branch points.

In one embodiment, the present invention provides methods of inducing neurite outgrowth in a subject including administering to the subject a therapeutically effective amount of a composition including R(−)-3,4-methylenedioxymethamphetamine (R(−)-MDMA). In some embodiments, the method is effective to lessen avoidance behavior in the subject.

In an embodiment, neurite outgrowth includes neurite number, neurite total length, number of neurite branch points per neuron or any combination thereof. In an embodiment, neurite outgrowth includes neurite outgrowth on prefrontal cortex neurons and/or hippocampal neurons.

In an embodiment, the R(−)-MDMA composition is administered at a dose about 1 mg/kg to 20 mg/kg R(−)-MDMA. In an embodiment, the stress-related disease or disorder comprises, for example, mood/depressive disorder, bipolar disorder, anxiety disorder, eating disorder, obsessive compulsive disorder, psychotic or delirium disorder, schizophrenia, schizoaffective disorder, personality disorder, abuse or neglect disorder, tic disorder, neurocognitive disorder, neurodevelopmental disorder, autism, autism spectrum disorder, learning disorder, disruptive mood regulation disorder, intermittent explosive disorder, antisocial personality disorder, conduct disorder, behavioral and psychological symptoms of dementia, depression, treatment resistant depression, anxiety, post-traumatic stress disorder (PTSD), and any combination thereof. In an embodiment, the stress-related disease or disorder is PTSD. In an embodiment, the stress-related disease or disorder is obsessive compulsive disorder. In an embodiment, the stress-related disease or disorder is an eating disorder.

### Neural Atrophy

It has been shown that chronic stress from conditions such as PTSD can cause neural atrophy and decrease the number of synapses within cortical and limbic circuits, which are associated with the regulation of mood, cognition, and behavior. It has been shown that chronic, but not acute, administration of typical antidepressant drugs, such as selective serotonin reuptake inhibitors (SSRIs), attenuate the effects of stress on neurogenesis and neuronal structure in animals and demonstrate antidepressant and anxiolytic effects in humans. Neural plasticity, and therefore neural atrophy, can be improved by the induction of neurite growth. Therefore, compounds that promote neural plasticity and neurite growth may have therapeutic benefit in the treatment of stress-related disorders.

As used herein “neuroplasticity” refers to any neuronal plasticity, which can include for example neurite outgrowth. Neurite outgrowth can broadly refer to various parameters than can be measured on neurites, including but not limited to neurite number, neurite total length, number of neurite branch point per neuron or any combination thereof. Neuronal plasticity in general and neurite outgrowth in particular can occur in any neurons and neurites in the brain, including but not limited to prefrontal cortex neurons and/or hippocampal neurons. As used herein, a “disease or disorder that can benefit from neuroplasticity” may include any disease or disorder that can be treated by, or than can see one or more of its symptoms alleviate by a change in neuroplasticity in the patient's brain, for example by neurite outgrowth, such as neurite outgrowth in prefrontal cortex neurons and/or hippocampal neurons.

In one embodiment, the present invention provides methods of treating neuronal atrophy in a subject including administering to the subject a therapeutically effective amount of a composition including R(−)-3,4-methylenedioxymethamphetamine (R(−)-MDMA) to lessen avoidance behavior in the subject.

In an embodiment, the administration of R(−)-MDMA induces neurite outgrowth. In an embodiment, neurite outgrowth includes neurite number, neurite total length, number of neurite branch points per neuron or any combination thereof. In an embodiment, neurite outgrowth includes neurite outgrowth on prefrontal cortex neurons and/or hippocampal neurons.

In an embodiment, the R(−)-MDMA composition is administered at a dose about 1 mg/kg to 20 mg/kg R(−)-MDMA. In an embodiment, the subject has a stress-related disease or disorder including depression, anxiety, post-traumatic stress disorder (PTSD), and any combination thereof. In an embodiment, the stress-related disease or disorder is PTSD.

### Neural Plasticity

Neuroplasticity is the ability of the brain to form and reorganize synaptic connections, especially in response to learning or experience or following an injury. It has been shown that exposure to stress causes a consistent suppression of neural plasticity. Therefore, traumatic events, such as events causing PTSD, can alter the neural connections and neural plasticity of the brain. However, the neuroplasticity can be used to mitigate the effects of PTSD. It has been shown that chronic, but not acute, administration of typical antidepressant drugs, such as selective serotonin reuptake inhibitors (SSRIs), attenuate the effects of stress on neurogenesis and neuronal structure in animals and demonstrate antidepressant and anxiolytic effects in humans. Neural plasticity can be improved by the induction of neurite growth. As such, compounds that promote neural plasticity may have therapeutic benefit in the treatment of stress-related disorders.

In one embodiment, the disclosure provides a method of inducing structural neuroplasticity in a subject including administering to the subject a therapeutically effective amount of a composition comprising or consisting of R(−)-3,4-methylenedioxymethamphetamine (R(−)-MDMA). In some embodiments the method is effective to lessen avoidance behavior in the subject.

In an embodiment, the administration of R(−)-MDMA induces neurite outgrowth. In an embodiment, neurite outgrowth includes neurite number, neurite total length, number of neurite branch point per neuron or any combination thereof. In an embodiment, neurite outgrowth includes neurite outgrowth on prefrontal cortex neurons and/or hippocampal neurons.

In an embodiment, the R(−)-MDMA composition is administered at a dose about 1 mg/kg to 20 mg/kg R(−)-MDMA. In an embodiment, the stress-related disease or disorder comprises, for example, mood/depressive disorder, bipolar disorder, anxiety disorder, eating disorder, obsessive compulsive disorder, psychotic or delirium disorder, schizophrenia, schizoaffective disorder, personality disorder, abuse or neglect disorder, tic disorder, neurocognitive disorder, neurodevelopmental disorder, autism, autism spectrum disorder, learning disorder, disruptive mood regulation disorder, intermittent explosive disorder, antisocial personality disorder, conduct disorder, behavioral and psychological symptoms of dementia, depression, treatment resistant depression, anxiety, post-traumatic stress disorder (PTSD), and any combination thereof. In an embodiment, the stress-related disease or disorder is PTSD. In an embodiment, the stress-related disease or disorder is obsessive compulsive disorder. In an embodiment, the stress-related disease or disorder is an eating disorder.

### Increasing BDNF Levels

Brain-Derived Neurotrophic Factor (BDNF) regulates many aspects of neuronal development and function in the nervous system. It is produced as proBDNF in response to neuronal activity or inflammatory stimulation and is then cleaved before associating into a homodimer. BDNF regulates neural stem cell survival and differentiation, axon/dendrite differentiation, synapse formation and maturation, and refinement of developing circuits. It is also a key regulator of synaptic plasticity and late-phase long-term potentiation. Studies in humans and animals also have shown that depression and stress exposure decrease cerebral cortex and hippocampal levels of brain-derived neurotrophic factor (BDNF), which promotes neuronal survival and synaptic plasticity. Therefore, BDNF may play a role in stress-related structural changes. It has been shown that chronic, but not acute, administration of typical antidepressant drugs, such as selective serotonin reuptake inhibitors (SSRIs), attenuate the effects of stress on neurogenesis, neuronal structure and BDNF levels in animals and demonstrate antidepressant and anxiolytic effects in humans. Therefore, BDNF may play a role in stress-related structural changes and compounds that increase BDNF levels may have therapeutic benefit in the treatment of stress-related disorders.

In one embodiment, the disclosure provides method of increasing brain-derived neurotrophic factor (BDNF) levels in a subject including administering to the subject a therapeutically effective amount of a composition including R(−)-3,4-methylenedioxymethamphetamine (R(−)-MDMA). In some embodiments the method is effective to lessen avoidance behavior in the subject.

In an embodiment, increasing BDNF levels includes increasing cerebral cortex and hippocampal BDNF levels. In an embodiment, increasing BDNF levels increases neuronal survival and synaptic plasticity.

In an embodiment, the R(−)-MDMA composition is administered at a dose about 1 mg/kg to 20 mg/kg R(−)-MDMA. In an embodiment, the stress-related disease or disorder comprises, for example, mood/depressive disorder, bipolar disorder, anxiety disorder, eating disorder, obsessive compulsive disorder, psychotic or delirium disorder, schizophrenia, schizoaffective disorder, personality disorder, abuse or neglect disorder, tic disorder, neurocognitive disorder, neurodevelopmental disorder, autism, autism spectrum disorder, learning disorder, disruptive mood regulation disorder, intermittent explosive disorder, antisocial personality disorder, conduct disorder, behavioral and psychological symptoms of dementia, depression, treatment resistant depression, anxiety, post-traumatic stress disorder (PTSD), and any combination thereof. In an embodiment, the stress-related disease or disorder is PTSD. In an embodiment, the stress-related disease or disorder is obsessive compulsive disorder. In an embodiment, the stress-related disease or disorder is an eating disorder.

### Pharmaceutical Compositions

As used herein, “pharmaceutical composition” refers to a formulation comprising an active ingredient, and optionally a pharmaceutically acceptable carrier, diluent or excipient. The term “active ingredient” can interchangeably refer to an “effective ingredient” and is meant to refer to any agent that is capable of inducing a sought-after effect upon administration. Examples of active ingredient include, but are not limited to, chemical compound, drug, therapeutic agent, small molecule, etc. In an embodiment, the active ingredient is R(−)-MDMA.

By “pharmaceutically acceptable” it is meant the carrier, diluent or excipient must be compatible with the other ingredients of the formulation and not deleterious to the recipient thereof, nor to the activity of the active ingredient of the formulation. Pharmaceutically acceptable carriers, excipients or stabilizers are well known in the art, for example Remington's Pharmaceutical Sciences, 16th edition, Osol, A. Ed. (1980). Pharmaceutically acceptable carriers, excipients, or stabilizers are nontoxic to recipients at the dosages and concentrations employed, and may include buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (for example, Zn-protein complexes); and/or non-ionic surfactants such as TWEEN™, PLURONICS™ or polyethylene glycol (PEG). Examples of carriers include, but are not limited to, liposome, nanoparticles, ointment, micelles, microsphere, microparticle, cream, emulsion, and gel. Examples of excipient include, but are not limited to, anti-adherents such as magnesium stearate, binders such as saccharides and their derivatives (sucrose, lactose, starches, cellulose, sugar alcohols and the like) protein like gelatin and synthetic polymers, lubricants such as talc and silica, and preservatives such as antioxidants, vitamin A, vitamin E, vitamin C, retinyl palmitate, selenium, cysteine, methionine, citric acid, sodium sulfate and parabens. Examples of diluent include, but are not limited to, water, alcohol, saline solution, glycol, mineral oil and dimethyl sulfoxide (DMSO).

In one embodiment, the disclosure provides a pharmaceutical composition including R(−)-MDMA and a pharmaceutically acceptable carrier. In an embodiment, the pharmaceutically acceptable carrier is saline or purified water. The pharmaceutical composition in accordance with the disclosure can be used in any of the methods disclosed herein.

The pharmaceutical compositions described herein can be formulated, for example, by employing conventional vehicles or diluents, as well as additives of a type appropriate to the mode of desired administration (for example, excipients, preservatives, etc.) according to techniques known in the art of pharmaceutical formulation. The pharmaceutical compositions described herein can also be formulated as is, without any carrier. The pharmaceutical compositions can be formulated in a variety of unit dosage forms depending upon the method of administration. Suitable unit dosage forms, include, but are not limited to powders, tablets, pills, capsules, lozenges, sprays, granules, etc.

The following examples are provided to further illustrate the embodiments of the disclosure and are not intended to limit the scope of the claims. Persons of skill in the will appreciate that other procedures, methodologies, or techniques generally known and available in the art may alternatively be used.

### EXAMPLES

### Example 1

### Evaluation of Mdma and Mda In Vitro Binding

Racemic MDMA and MDA as well as their individual enantiomers (i.e., R(−)-MDMA. S(+)-MDMA, R(−)-MDA and S(+)-MDA) were evaluated for in vitro receptor binding.

Each of the compounds was evaluated for binding to 87 different targets as part of a panel (Eurofins Panlabs, Taiwan, China). Each compound was tested at a single concentration of 10 mM. The panel included mostly G-protein-couple receptors, ion channels, enzymes, transporters and nuclear receptors, expressed in the central nervous system, cardiovascular, respiratory, gastrointestinal and renal systems. A limited number of targets were of pig, rat or guinea pig origin. Targets were expressed in human recombinant cells lines, rat brain or spinal cord, guinea pig brain or pig heart. Methods were adapted from the scientific literature to maximize reliability and reproducibility. Reference standards were run as an integral part of each assay to ensure validity of the results obtained (available online at the eurofins website for the safety screen 87 panel).

In additional experiments, the concentration-dependent binding of R-MDMA and S-MDMA at selected targets were evaluated in the same Eurofins Panlabs radioligand binding assays as included in the prior broad panel testing at a single concentration. Each compound was tested at 10 half-log unit concentrations up to 10 μM (i.e., 0.3 nM to 10 μM), in duplicate.

Targets with significant inhibition of radioligand binding or enzymatic activity (≥50% inhibition) at 10 μM for each test article are summarized Table 1. The value in the table reflects the % inhibition of binding or activity, ≥50%, for the specific target receptor at 10 μM of each compound. Cells with no values indicate the % inhibition of binding or activity for that target receptor was <50% at 10 μM of that compound.

At 10 μM, racemic MDMA significantly bound to serotonin 5-HT_{2A}, 5-HT_{2B} and 5-HT_{2C} receptors, as well as the nicotinic acetylcholine a3b4 ion channel. R(−)-MDMA significantly bound to 5-HT_{2B} and 5-HT_{2C} receptors, nicotinic acetylcholine a3β4 ion channels, and the L-type calcium channel phenylalkylamine binding site. S(+)-MDMA significantly bound to the L-type calcium channel phenylalkylamine site. Racemic MDA significantly bound to 5-HT_{2B} and 5-HT_{2C} receptors, as well as adrenergic α2A receptors. R(−)-MDA significantly bound to 5-HT_{2B} and 5-HT_{2C} receptors, and 5-HT_{1A} receptors. S-MDA significantly bound to 5-HT_{2B} and 5-HT_{2C} receptors, as well as adrenergic α2A and α2B receptors.

Targets associated with significant binding at 10 μM included 5-HT_{2B} and 5-HT_{2C} receptors for all compounds except S(+)-MDMA. The R(−) enantiomer of MDMA and the S(+)-enantiomer of MDA exhibited more similar binding profiles to their respective racemates.

TABLE 1

| | | ≥50% inhibition of binding or activity at 10 uM | | | | | |
|---|---|---|---|---|---|---|---|
| | | Racemic | | | Racemic | | |
| Species | Target Receptor | MDMA | R-MDMA | S-MDMA | MDA | R-MDA | S-MDA |
| Human | Serotonin 5-HT2A | 59 | | | | | |
| Human | Serotonin 5-HT2B | 85 | 79 | | 89 | 88 | 82 |
| Human | Serotonin 5-HT2C | 87 | 78 | | 74 | 76 | 53 |
| Human | Serotonin 5-HT1A | | | | | 50 | |
| Human | Adrenergic α2A | | | | 53 | | 63 |
| Human | Adrenergic α2B | | | | | | 51 |
| Human | Nicotinic acetylcholine | 66 | 55 | | | | |
| | α3β4 | | | | | | |
| Rat | L-type calcium channel | | 55 | 51 | | | |
| | (pheylalkyamine) | | | | | | |

In additional binding experiments, the concentration-dependent binding of R-MDMA and S-MDMA were evaluated at selected targets for which either of the enantiomers exhibited >50% inhibition of binding for that target at 10 μM (i.e., 5-HT_{2B}, 5-HT_{2C}, nicotinic acetylcholine α3β4, and L-type calcium channel phenylalkylamine binding site) as well as at the 5-HT_{2A} receptor. The results are shown in Table 2. R-MDMA exhibited higher potency/affinity binding than S-MDMA at 5-HT_{2A}, 5-HT_{2B} and 5-HT_{2C} receptors. R-MDMA and S-MDMA showed similar potency/affinity binding at L-type calcium channel phenylalkylamine binding site. Both compounds also showed relatively weak binding at nicotinic acetylcholine α3β4 channels, based on <50% inhibition of radioligand binding when tested at concentrations ranging from 0.3 nM to 10 μM.

TABLE 2

| | | IC50 (uM) | | Ki (uM) | |
|---|---|---|---|---|---|
| | | R- | S- | R- | S- |
| Species | Target Receptor | MDMA | MDMA | MDMA | MDMA |
| Rat | L-type calcium channel | 9.5 | 9.31 | 9.23 | 9.05 |
| | (pheylalkyamine) | | | | |
| Human | Serotonin 5-HT2A | 9.91 | N.C. | 2.83 | N.C. |
| Human | Serotonin 5-HT2B | 1.4 | 12 | 0.89 | 7.65 |
| Human | Serotonin 5-HT2C | 2.6 | N.C. | 1.36 | N.C. |
| Human | Nicotinic | N.C. | N.C. | N.C. | N.C. |
| | acetylcholine α3β4 | | | | |

These results indicate similarities and differences among racemic, R(−) and S(+)-MDMA and MDA. Targets associated with significant binding at 10 μM included 5-HT_{2B} and 5-HT_{2C} receptors for all compounds except S(+)-MDMA, demonstrating the distinct pharmacological effects of this compound.

R(−)-MDMA exhibited a more similar binding profile than S(+)-MDMA to racemic MDMA. This indicates that the R(−)-MDMA enantiomer would show more similar pharmacological effects to racemic MDMA. As racemic MDMA has shown efficacy in a Phase 3 clinical trial for PTSD, the more similar binding profile of R(−)-MDMA indicates the potential for similar on-target therapeutic-like activity with more limited off-target activity than the racemate due to a lack of S-MDMA-mediated effects, which can translate into a therapeutic with a superior efficacy and/or safety profile.

Follow-up concentration-response radioligand binding studies indicated that R-MDMA binds to 5-HT_{2A}, 5-HT_{2B} and 5-HT_{2C} receptors with greater potency/affinity than S-MDMA. These findings indicate that therapeutic-like and/or side effects of racemic MDMA that involve these receptors may be reproduced by administration of the single enantiomer R-MDMA and not by administration of similar doses of the single enantiomer S-MDMA.

### Example 2

### Effect of MDMA and MDA on Neurite Growth

Racemic MDMA and MDA as well as their individual enantiomers (i.e., R(−)-MDMA, S(+)-MDMA, R(−)-MDA and S(+)-MDA) were evaluated for the ability to induce neurite growth.

Female Wistar rats of 17 days gestation were killed by cervical dislocation and the fetuses (typically 6 to 8 in number) were removed from the uteri. Fetal brains were placed in ice-cold medium of Leibovitz (L15, Gibco, France). Cortex was dissected and meninges were carefully removed. The cortical neurons were dissociated by trypsinization (trypsin-EDTA, Gibco) in the presence of DNAse I (Roche, France). The reaction was stopped by addition of Dulbecco's Modified Eagle Medium (DMEM; Gibco) with 10% of fetal bovine serum (FBS; Gibco). The suspension was triturated with a 10-ml pipette and a 21-gauge needle syringe and centrifuged. The pellet of dissociated cells was resuspended in a medium consisting of Neurobasal (Gibco) supplemented with 2% B27 supplement (Gibco), 0.5 mM L-Glutamine (Gibco), and an antibiotic-antimycotic mixture. Viable cells were counted in a Neubauer cytometer and cells were seeded in 96-well plates (Costar) precoated with poly-L-lysine at 10,000 cells/well. Compounds, including negative control (vehicle), positive control (Donepezil 250 nM), and test article(s) at one or more concentrations, were added to the cultures on the plating day (Day 0). Stock solutions were prepared in sterile water at 10 mM and stored at −20° C. until used. Further dilutions were prepared in culture in the medium on the day of the treatment. 0.1% Sterile water was present in all tested conditions. The following conditions were tested: 1) Racemic MDMA (10000, 1000, 100, 10, 1, 0.1, 0.01, 0.001 nM), 2) S(+)-MDMA (10000, 1000, 100, 10, 1, 0.1, 0.01, 0.001 nM), 3) R(−)-MDMA (10000, 1000, 100, 10, 1, 0.1, 0.01, 0.001 nM), 4) Racemic MDA (10000, 1000, 100, 10, 1, 0.1, 0.01, 0.001 nM), 5) S(+)-MDA (10000, 1000, 100, 10, 1, 0.1, 0.01, 0.001 nM), and 6) R(−)-MDA (10000, 1000, 100, 10, 1, 0.1, 0.01, 0.001 nM).

The experimental protocol was performed in two independent cultures (i.e., from two different pregnant rats). For each culture, each condition was tested in sextuplet (6 wells per condition per culture for a total of 12 wells per condition). Each plate contained three types of experimental conditions: the negative control condition treated with vehicle (0.1% sterile water), the positive control condition treated with Donepezil (250 nM) and test article conditions. After three days of plating and compound treatment (Day 3), cultures were fixed with paraformaldehyde in phosphate buffered saline at 4° C. (PBS, 4%, Sigma). All subsequent steps were performed at room temperature. Cells were successively permeabilized for 30 minutes using 0.1% triton, saturated with PBS containing 3% bovine serum albumin (BSA) and incubated for 1 hour with anti-beta III tubulin antibody (T5168, Sigma). Cells were first washed three times and then were incubated for 1 hour with goat anti-mouse secondary antibody coupled with Alexa Fluor 488 (AF488, Invitrogen A11001). Finally, nuclei were stained with 4′-6-diamidino-2-phenylindole (DAPI). After rinsing the cells with PBS, the plate was imaged and neurite networks were examined and analyzed using a High-Content Screening platform (CellInsight CXS, Thermo Scientific) with integrated Photometrics high-resolution fluorescent camera, Olympus objective (10×) and HCS Studio Cell Analysis Software. Approximately 2,000 neurons per well were analyzed using a validated cortex neuron outgrowth algorithm that utilizes parameters optimized for analysis of embryonic rat cortical neuron cultures. The HCS Studio Cell Analysis Software output included values for neurite total count (#), neurite total length (μm) and total number of branch points (#) for each of the 6 wells per culture. The evaluation of neurite outgrowth was performed using the average number of neurites per neuron (#), the average of total neurite length per neuron (μm) and the average number of branch points per neuron (#) across 12 wells. Data also was transformed to percent (%) of the average vehicle control value. Results were expressed as mean (±s.e.m.) of the transformed (% of vehicle control) data. Statistical analyses were performed on transformed percent of the average vehicle control data for each measure for each test article using one-way analysis of variance (Anova, StatView). Where applicable, Dunnett's test was used for multiple pairwise comparisons to the negative control (vehicle) condition. Transformed percent of the average vehicle control data for each measure for the positive control Donepezil (250 nM) was compared to the negative control (vehicle) condition using an unpaired t test. The level of significance was set at p value less than or equal to 0.05 (*p<0.05).

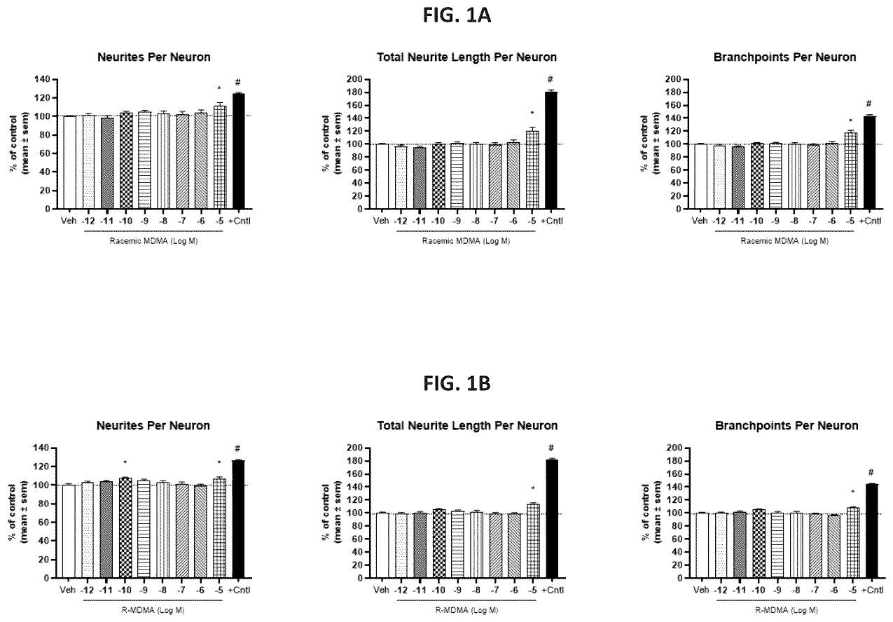

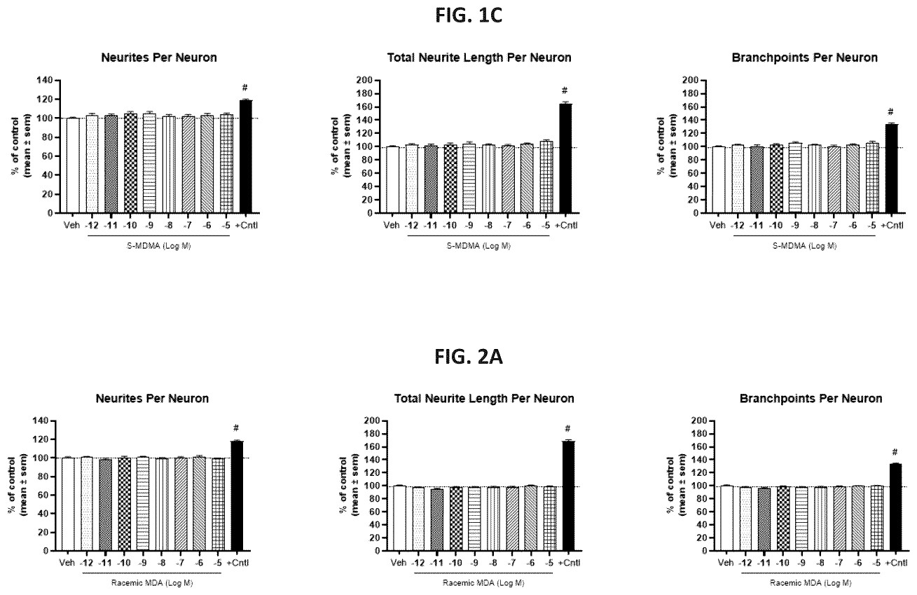

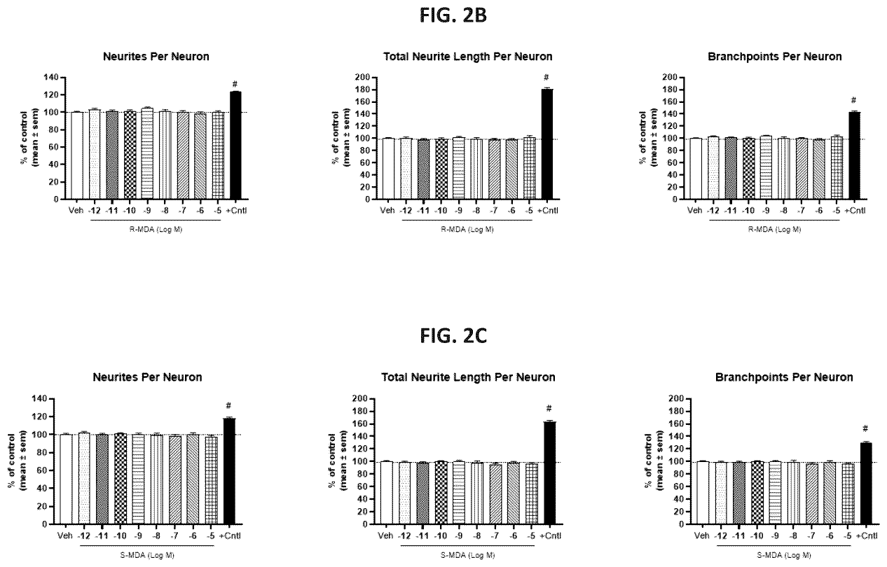

Under the conditions tested, in each of the 6 test article experiments, the positive control Donepezil (250 nM) demonstrated the expected effects of significantly increasing neurite number (#), neurite total length (μm) and total number of branch points (#) compared to the 0.1% sterile water vehicle control. Racemic R,S(+/−)-MDMA significantly increased neurite number (#), neurite total length (μm) and total number of branch points (#) at 10 μM compared to the 0.1% sterile water vehicle control (FIG. 1A). R(−)-MDMA significantly increased neurite number (#), neurite total length (μm) and total number of branch points (#) at 10 μM, and also significantly increased neurite number (#) at 0.0001 μM, compared to the 0.1% sterile water vehicle control (FIG. 1B). S(+)-MDMA, racemic R,S(+/−)-MDA, R(−)-MDA and S(+)-MDA did not significantly alter neurite number (#), neurite total length (μm), or total number of branch points (#) at any tested concentration compared to the 0.1% sterile water vehicle control (FIGS. 1C, 2A, 2B and 2C respectively).

Of the six compounds tested, racemic R,S(+/−)-MDMA and R(−)-MDMA significantly increased neurite outgrowth parameters under the conditions evaluated. Racemic and R(−)-MDMA increased all three parameters measured at 10 μM. In contrast, S(+)-MDMA, and racemic MDA, R(−)- and S(+)-MDA, did not significantly increase any of the neurite parameters measured. These data show that racemic MDMA and R(−)-MDMA exhibit structural neuroplasticity, which will have therapeutic benefit in the treatment of stress-related disorders.

### Example 3

### Effect of MDMA and MDA on Head Twitch Response

Racemic MDMA and MDA as well as their individual enantiomers (i.e., R(−)-MDMA, S(+)-MDMA, R(−)-MDA and S(+)-MDA) were evaluated for head twitch response (HTR).

Male C57BL/6J mice at 6-8 weeks of age were purchased from Jackson Laboratories and housed four per cage in a climate and humidity-controlled room in a vivarium at the University of California, San Diego. The room operated on a reverse light cycle (lights on at 1900 h, off at 0700 h) with food and water available ad libitum, except during testing. All testing was performed during 1000 h and 1800 h. After a minimum of 1 week acclimation to the housing facility, mice were anesthetized using a mixture of ketamine (100 mg/kg IP) and xylazine (10 mg/kg IP). Under deep anesthesia, an incision was made in the scalp and a small neodymium magnet (4.57 mm×4.57 mm×2.03 mm) was attached to the cranium using cyanoacrylate and dental cement. After at least a 2-week recovery period, the mouse was removed from the home cage and injected intraperitoneally with the test article or vehicle (0.9% sterile saline). Immediately, the mouse was placed in a glass cylinder surrounded by a magnetometer coil and the head twitch response (HTR) was assessed for 30 minutes (Halberstadt and Geyer, 2013). The output of the coil was recorded using a Powerlab/8SP with LabChart v.7.3.2 (ADInstruments). Coil voltage was amplified, low-pass filtered (2-10 kHz cut-off frequency) to remove interference, digitized and sampled at 20 kHz. Head twitches were identified using a validated technique based on artificial intelligence (Halberstadt, 2020). Events in the recordings were transformed into a visual representation in the time-frequency domain and deep features were extracted using the pretrained convolutional neural network ResNet-50, and the images were subsequently classified using a Support Vector Machine algorithm.

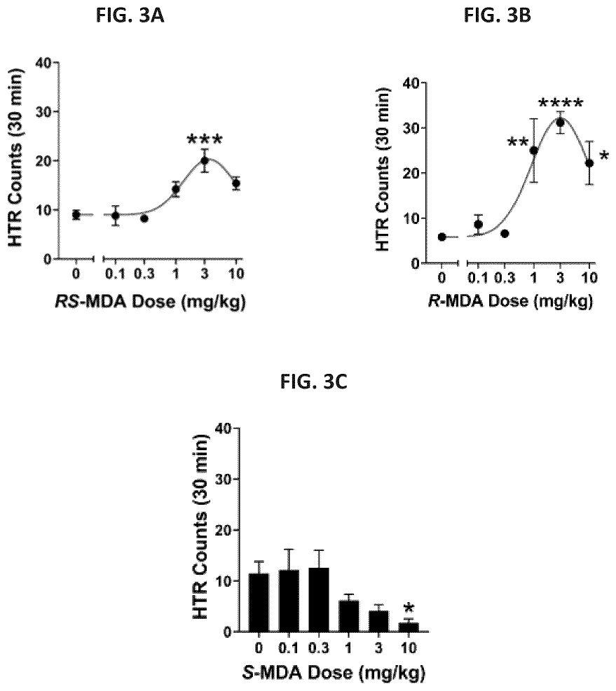

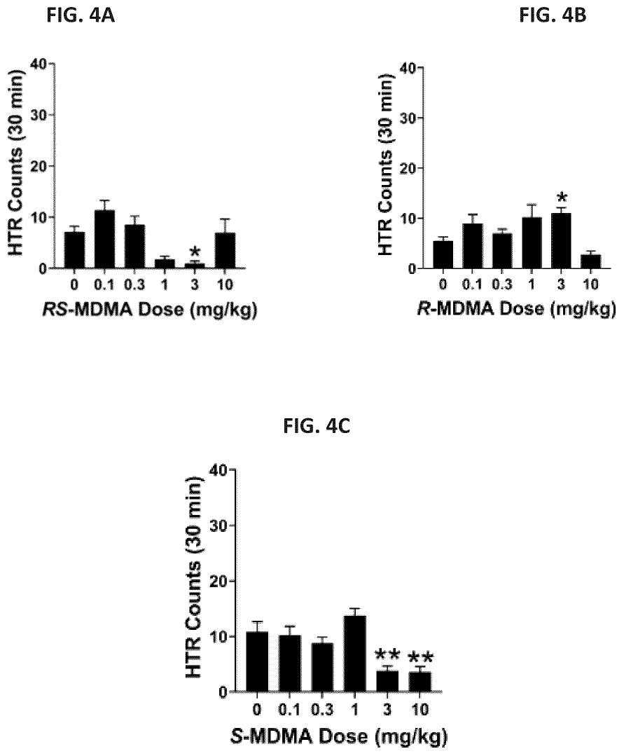

Test articles (R,S-MDA, R(−)-MDA, S-(+)MDA), R,S-MDMA, R(−)-MDMA and S(+)-MDMA) were supplied by the NIDA Drug Supply Program as neat powders. Racemic MDA and MDMA and their enantiomers were dissolved in saline and injected IP at a volume of 5 mL/kg. Each dose was calculated based on the salt form of the compounds. For each treatment condition, six groups of mice (n=5-6/group) were treated with vehicle or the test drug (0.1, 0.3, 1, 3, or 10 mg/kg) and HTR assessed for 30 minutes immediately following injections. To avoid carryover effects, each treatment condition was performed at least seven days apart. Total number of mice used for the experiment was 63. The results are shown in FIGS. 3A-C and 4A-C.

Administration of R(−)-MDA at doses of 1, 3, and 10 mg/kg elicited an increase in HTR, while the racemic R,S-MDA increased HTR at 3 mg/kg compared to control (vehicle). In contrast, the highest dose of 10 mg/kg S-MDA showed a reduction of HTR compared to control. Administration of 3 mg/kg R(−)-MDMA yielded an increase in HTR, but the same dose of 3 mg/kg R,S-MDMA elicited a decrease in HTR compared to control. The 3 and 10 mg/kg doses of S(+)-MDMA exhibited a reduction of HTR compared to control. Asterisks in the figures indicate significant differences compared to the control (vehicle) condition, *p<0.05, **p<0.01, ***p<0.001, ****p<0.0001.

These mouse HTR data show that in vivo administration of R(−)-MDMA and its primary metabolite (R(−)-MDA) activates the 5-HT_{2A} receptor in a manner similar to classical serotonergic psychedelic drugs more effectively than the racemate or the S isomer of MDMA. The induction of the HTR is most likely achieved via direct agonist action of 5-HT_{2A} receptors, which parallels most classical psychedelics. The S isomers of both MDMA and MDA attenuated the HTR showing that their activity is an indirect 5-HT agonist, activating other 5-HT receptor subtypes that may oppose or suppress its 5-HT_{2A} receptor-mediated effects. These results show that targeted stimulation of 5-HT_{2A} receptors in a manner similar to classical serotonergic psychedelic drugs contributes to the in vivo effects of R-MDMA and, thus, may be involved in its potential therapeutic-like activity, such as in the treatment of PTSD and other stress-related disorders.

### Example 4

### Effect of MDMA on Extinction of Fear Memory

Racemic MDMA and R(−)-MDMA were evaluated for extinction of fear memory. The study utilized a mouse extinction of conditioned fear assay. A conditioned fear response is induced by pairing a previously neutral stimulus (conditioned stimulus; CS, such as an auditory tone) and aversive stimulus (unconditioned stimulus; US, such as food shocks). The fear response is later extinguished with repeated re-exposure to the CS in the absence of the US. This is a useful model for understanding learning and memory processes required for long-term recovery from PTSD, because it resembles exposure-based therapy, wherein repeated exposure to a fear-eliciting stimulus or memory promotes a reduced fear response to future reminders of the trauma. Studies in mice demonstrated that single administration of racemic MDMA or R(−)-MDMA facilitated extinction of conditioned fear. The current experiment investigated the ability of varying doses of R(−)-MDMA to facilitate extinction of conditioned fear in mice.

Methods: Male C57BL/6 mice (n=88) from Jackson Laboratories (10-12 weeks of age) were assigned randomly to treatment groups. Animals were acclimated to the laboratory for at least five days prior to experimental manipulation. Mice were housed on a 12 hr light/dark cycle (lights on 7:00 AM) with no more than four mice per cage on a ventilated cage rack system. Standard rodent chow and water was available ad libitum. Animals were maintained in a controlled environment, and temperature and humidity were recorded continuously in the holding room.

The animal procedures were conducted according to established protocols approved by the IACUC committee and Melior Discovery Inc. Standard Operation Procedures (SOP). The behavioral procedure was adapted from Young et al., Transl Psychiatry 5, e634 (2015) with slight modifications. Mice were handled for two consecutive days for four minutes each day and injected with drug vehicles prior to the experiment. On each day of the experiments, animals were acclimated to a procedure room adjacent to the fear conditioning room for at least 30 minutes prior to the start of experimental sessions each day. Fear conditioning was conducted in individual automated chambers built by Kinder Scientific (Poway, CA) that detect movement with infrared beams. On Day 1, Conditioning, each animal was placed in a chamber and presented a training session (Context A). An almond scent was present under the grid floor during the entire session. The training procedure consisted of a 2-minute habituation followed by a 30-second, 80 dB tone (CS). During the last 2 seconds of the tone, the animal received a 1 mA foot shock (US). This US-CS procedure occurred once for a total of one paired presentation of tone and shock. Freezing behavior (immobility) was recorded in 5-second intervals during the session. Percent baseline freezing behavior was determined in the 2-minute habituation period. After the session, animals were returned to their cages in the holding room. Animals were not manipulated on Day 2. On Day 3, Extinction Training, two days (48 hours) following fear conditioning, each animal was placed in Context B, in which the tactile and odor cues of the conditioning chambers were altered with a different (lemon) scent and black Plexiglas floor placed over the grid floor. Following a 2-minute acclimation period, the animal received four 30-second presentations of the CS (tone) in the absence of the US (foot shock), with a 45-second interval between presentations and 30 seconds of chamber consolidation. Immobility was recorded in 5-second intervals to measure baseline freezing response to the altered context, and freezing response to each tone/cue presentation. On Day 3, each animal was administered vehicle or racemic MDMA by intraperitoneal (IP) injection at 30 minutes prior, to the extinction training session. On Days 4, 8, and 13, Extinction Testing sessions, each animal was placed in Context B. Following a 2-minute acclimation period, each animal received four 30-second presentations of the CS (tone) in the absence of the US (foot shock), with a 45-second interval between presentations and 30 seconds of chamber consolidation. Immobility was recorded in 5-second intervals to measure baseline freezing response to the altered context, and freezing response to each tone/cue presentation.

Animals were randomized to treatment groups based on their responses to conditioning on Day 1, to ensure that animals in each condition exhibited a similar degree of freezing behavior prior to test article administration. Each animal in each treatment group received IP administration of vehicle or R(−)-MDMA or racemic MDMA at 30 minutes prior to extinction training on day 3. There were a total of 5 treatment groups with n=15 mice/group, including 1) vehicle, 2) 7.8 mg/kg racemic MDMA, 3) 10 mg/kg R-MDMA, 4) 17 mg/kg R-MDMA, and 5) 30 mg/kg R-MDMA. R-MDMA (10, 17 or 30 mg/kg) or racemic MDMA (7.8 mg/kg) was formulated in 0.9% Saline. Injection volumes were 10 mL/kg. The study was run in 2 cohorts. Data were presented as mean±standard error of the mean of the percentage (%) of the session time spent freezing (absence of movement other than respiration) or activity counts during the 2-minute acclimation period. Freezing and activity data were analyzed statistically using 2-way analysis of variance (ANOVA) with Day/session as the within-subjects factor and treatment as the between-subjects factor. Following a significant overall ANOVA (p<0.05), post hoc comparisons between the vehicle and each treatment condition were made using Fisher's Least Significant Difference test (*p<0.05, **p<0.01, ****p<0.001).

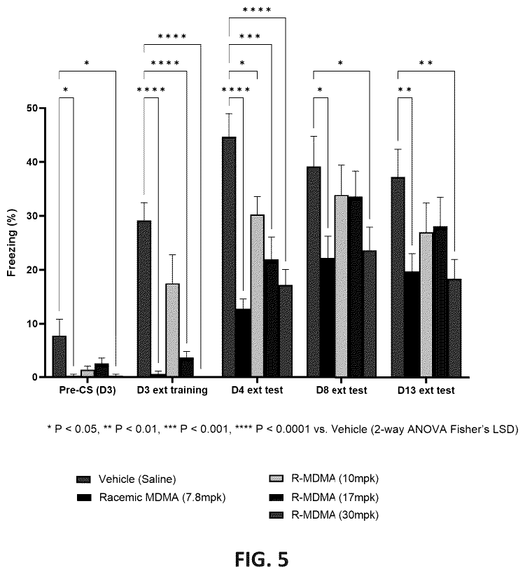

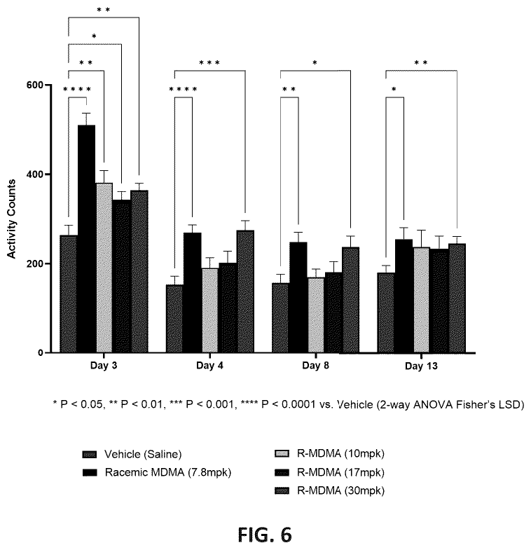

Over extinction training (Day 3) and testing (Days 4, 8 and 13) sessions, all doses of R-MDMA (10, 17 and 30 mg/kg) and racemic MDMA (7.8 mg/kg) exhibited significant effects on freezing (FIG. 5) and/or activity (FIG. 6) measures.

On Day 3, the day of single compound administration, R-MDMA (17 and 30 mg/kg) reduced freezing, as indicated by significant responses from the vehicle control group. There was no significant effect on freezing with 10 mg/kg R-MDMA. Significantly increased activity counts were seen with all doses of R-MDMA tested compared to vehicle control. However, unlike the effect on freezing, the effect of R-MDMA on activity did not appear to be dose-dependent. On Day 3, racemic MDMA (7.8 mg/kg) also significantly reduced freezing with a concomitant significant increase in activity.

On Day 4, all treatment groups (10, 17 and 30 mg/kg R-MDMA and 7.8 mg/kg racemic MDMA) showed significantly reduced freezing compared to vehicle, with only 30 mg/kg R-MDMA and racemic MDMA showing significantly increased activity.

On Days 8 and 13, animals previously treated with single administrations of 30 mg/kg R-MDMA or 7.8 mg/kg racemic MDMA continued to show significantly reduced freezing with concomitant significant increases in activity. As shown in FIG. 5 freezing (%) across Days 3, 4, 8 and 13 (Extinction Training and Testing Sessions) was measured in all Groups.

Together, these results show that single administration of 30 mg/kg R-MDMA exhibited a profile of durable facilitation of extinction of fear learning across each of the 4 test days, similar to the effects of single administration of 7.8 mg/kg racemic MDMA. Both 30 mg/kg R-MDMA and 7.8 mg/kg racemic MDMA also significantly increased locomotor activity across each of the 4 test days. However, the data indicate that 30 mg/kg R-MDMA exhibits less locomotor activation on the day of single administration, compared to 7.8 mg/kg racemic MDMA.

The results also show the therapeutic potential of single administration of lower R-MDMA doses. Freezing was significantly reduced on the days of and following single dosing of 17 mg/kg R-MDMA (Days 3 and 4), but not on Days 8 and 13, indicating a more time-limited therapeutic-like facilitation of extinction of fear learning. This dose of R-MDMA significantly increased locomotor activity only on the day of administration, indicating that the durable therapeutic-like effect on freezing observed on Day 4 was not due in any part to increased activity. Single administration of 10 mg/kg R-MDMA also significantly reduced freezing on the day after dosing (Day 4), without a significant increase in activity, indicating that this therapeutic-like effect was not due in any part to increased activity. The more limited time-course of significant facilitation of extinction of fear learning observed at these lower R-MDMA doses indicate the potential for durability of therapeutic-like efficacy to be dose-dependent.

A surprising finding in the current study is that each of the 3 tested doses of R-MDMA (10, 17, 30 mg/kg IP) significantly increased locomotor activity compared to the vehicle control group following single administration on Day 3. Also surprising, the effect of R-MDMA on activity was not dose-dependent (i.e., there was a relatively flat response across tested doses), in contrast to its clear dose-dependent effects on freezing behavior. Sigmoidal dose-response functions typically are observed when drugs interact with their molecular targets/receptors.

It also was surprising that the significant increase in activity relative to the vehicle group observed on Day 3 following single administration of racemic MDMA (7.8 mg/kg) and R-MDMA (30 mg/kg) was maintained in the absence of additional drug administration on Days 4, 8, and 13.

Based on the pharmacological activities within the single enantiomer of R-MDMA, this compound has the potential for reduced side effects, such as hyperthermia and increased heart rate and blood pressure, relative to racemic MDMA or S-MDMA. The current study data show that R-MDMA exhibits therapeutic-like activity in a mouse extinction of fear learning assay similar to racemic MDMA. This indicates that R-MDMA has a similar therapeutic profile compared to racemic MDMA for the treatment of stress-related disorders, such as PTSD.

### Example 5

### Study of the Effect of R(−)-MDMA on Mouse Locomotor Activity In Vivo

This example studies dose-related effects of single administration of R-MDMA on measures of activity, including horizontal locomotion and vertical rearing, over time in mice, in comparison to a reference dose of racemic MDMA.

Methods. Male C57BL/6 mice (n=10-12 per group) from Jackson Laboratories, US (10-12 weeks of age), were assigned randomly to treatment groups. The animals were acclimated to the laboratory for at least five days prior to experimental manipulation. Mice were housed on a 12 hr light/dark cycle (lights on 7:00 AM) with no more than 4 mice per cage on a ventilated cage rack system. Standard rodent chow and water were available ad libitum, except during experimental test sessions. Animals were maintained in a controlled environment, and temperature and humidity were recorded continuously in the holding room.

The animal procedures were conducted according to established and approved protocols. On each experimental day, animals were acclimated to a procedure room adjacent to the testing room for at least 30 minutes prior to the start of experimental sessions. To evaluate acute compound effects on activity, an animal was administered a single dose of test article 30 minutes prior to individual placement in a novel test chamber on experimental day 1. Activity was detected using photo beams (e.g., 16×16 beams with a space of 2 cm between beams, Med Associates®) and recorded using the Activity Monitor™ program designed by Med Associates®. The primary endpoint was total distance traveled (cm), as a measure of locomotion. Additional activity measures included total ambulatory counts, ambulatory time (sec), vertical counts and vertical time (sec). All endpoints were recorded and analyzed over a 30-minute test period. At the end of the test session, each animal was returned to its home cage. To evaluate potential lasting effects on activity following single administration of compounds, each animal was re-evaluated in the same test chamber on experimental days 2, 6, and 11, without additional test article administration.

Test conditions included single administration of vehicle, R-MDMA (10, 17, 30 mg/kg), or racemic MDMA (7.8 mg/kg). R-MDMA or racemic MDMA was formulated in 0.9% saline on the day of administration and given via the intraperitoneal (IP) route in a 10 mL/kg injection volume. Animals were randomly assigned to treatment groups and testing was conducted in a manner counterbalancing treatment groups. Activity measures were analyzed statistically using 2-way analysis of variance (ANOVA) with experimental day as the within-subjects factor and treatment as the between-subjects factor. Following a significant overall ANOVA (p<0.05), post hoc comparisons between test article and vehicle conditions were made using Fisher's Least Significant Difference (LSD, p<0.05).

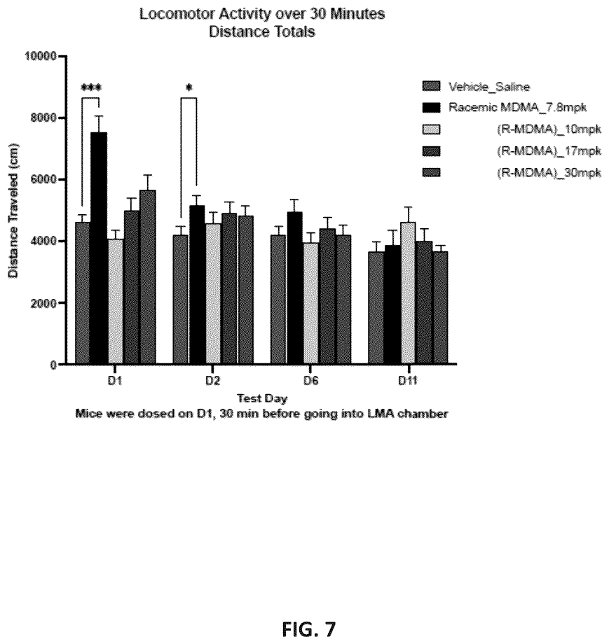

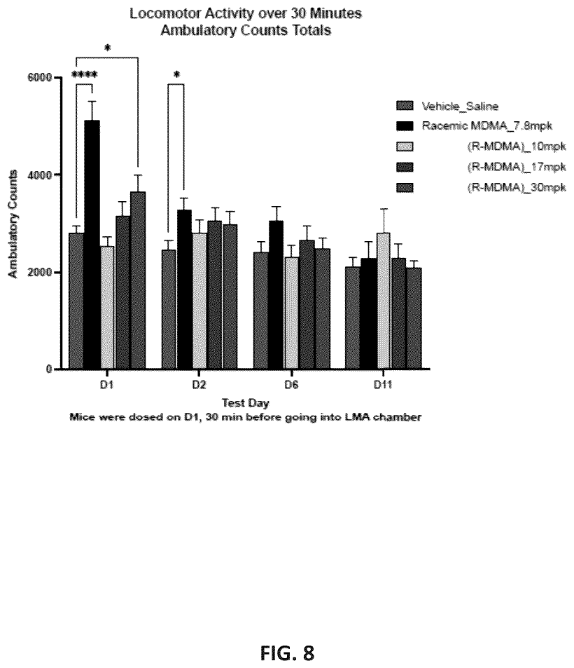

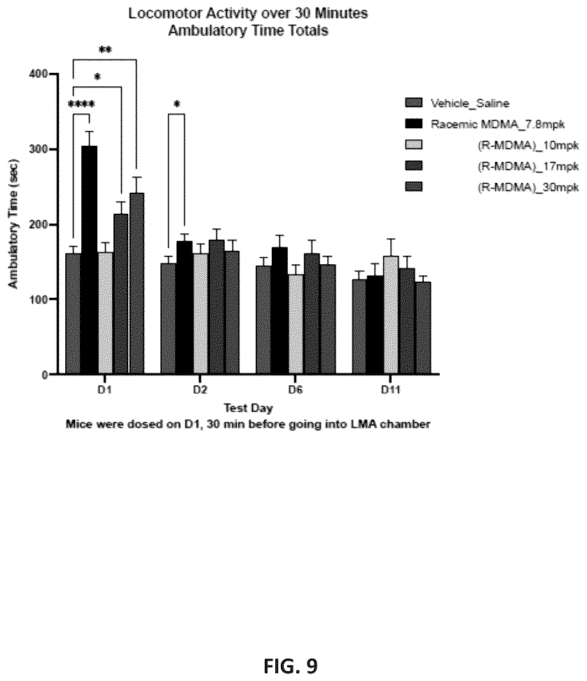

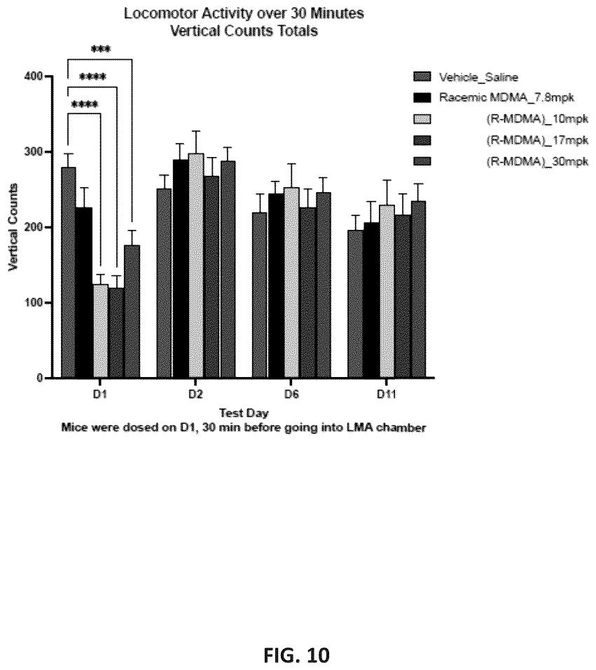

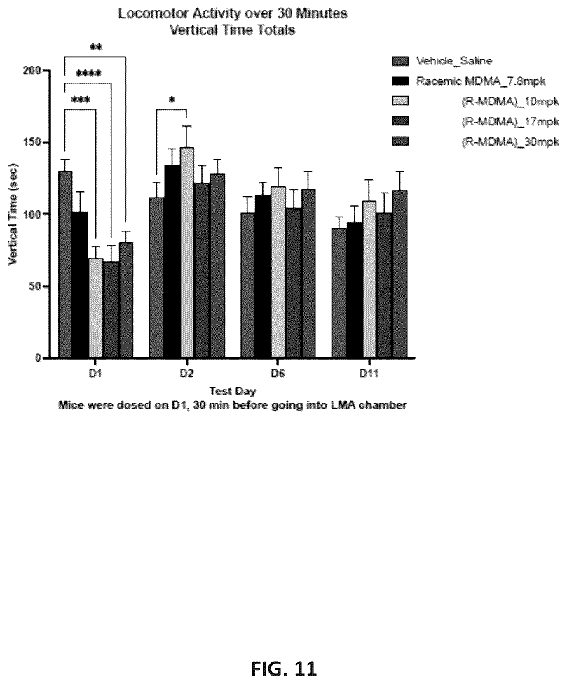

Results. The data indicated that racemic MDMA significantly increased the primary measure, total distance traveled (FIG. 7), on the day of administration (Day 1) and this increase persisted out to Day 2. Racemic MDMA also increased total ambulatory counts and time (shown in FIG. 8 and FIG. 9, respectively), on the day of administration (Day 1) and these increases persisted out to Day 2. Racemic MDMA did not alter total vertical counts (FIG. 10) or total vertical time (FIG. 11) on any day of testing.

The data also indicated that R-MDMA exhibited no significant increase in the primary measure, total distance traveled, at any dose or on any day of testing (FIG. 7). R-MDMA significantly increased total ambulatory counts (FIG. 8) only on Day 1 at the highest dose, and significantly increased total ambulatory time (FIG. 9) only on Day 1 at the 2 highest doses. R-MDMA exhibited significantly reduced total vertical counts (FIG. 10) and total vertical time (FIG. 11) at all 3 dose levels only on Day 1. Day 1 effects of R-MDMA were dose-related. As illustrated in FIG. 11, on day 2 only, the lowest dose of R-MDMA exhibited a significant increase in total vertical time.

Conclusions. The data demonstrated that racemic MDMA and R-MDMA generate observable differences in locomotor activity. For example, R-MDMA (unlike racemic MDMA) did not significantly increase the primary measure, total distance traveled, at any dose up to 30 mg/kg or on any test day up to Day 11 post single administration (FIG. 7), indicating that the R-enantiomer did not induce significant acute (Day 1) or persistent (Day 2, Day 6, Day 11) hyperactivity. These Day 1 results with R-MDMA and racemic MDMA build on and are in general agreement with reports from others (see, e.g., Curry et al., 2018). For the secondary activity measures that were increased on Day 1 for both racemic MDMA and R-MDMA (i.e., total ambulatory counts (FIG. 8) and ambulatory time (FIG. 9)), the data showed that the magnitude of the locomotor stimulation was greater and longer lasting following racemic MDMA administration. This data shows that a single administration of R-MDMA (up to 30 mg/kg) does not induce a robust and significant acute or persistent hyperactivity profile, which stands in contrast to the hyperactivity profile that was observed with a lower dosage of racemic MDMA (7.8 mg/kg). As such, the data demonstrates a therapeutic advantage that the R-MDMA enantiomer holds over at least the racemate, if not also the S-MDMA enantiomer. Furthermore, in the context of the mouse extinction of fear learning study results, which are described in Example 4 and relevant to the treatment of stress-related disorders such as PTSD, these results indicate that the significant and durable therapeutic-like effects of R-MDMA were not due simply to increased activity.

### Example 6

Neurobehavioral and Physiological Effects of Racemic, S(+)- and R(−)-MDMA in Rats

This example evaluates acute neurobehavioral and physiological effects of racemic MDMA, and its stereoisomers, R-MDMA, and S-MDMA, when cumulatively administered via subcutaneous (SC) injections to telemeterized adult male CD® (Sprague Dawley) rats. The observations were assessed through a rat functional observation battery (FOB) and continuous physiological measurements on the day of cumulative dosing, which are provided in further detail below.

Methods. A total of 42 adult male CD® (Sprague Dawley) rats (Charles River Laboratories) were surgically implanted with DSI radiotelemetry devices by personnel at Charles River Laboratories Raleigh, NC, per their test site SOPs. Telemetry devices supported continuous recording of temperature, activity, blood pressure and heart rate. Telemeterized rats were shipped to the Charles River Laboratories testing facility (Mattawan, MI) and acclimated to the environment for at least 1 week prior to experimentation. Animals were pair-housed in solid bottom cages with nonaromatic bedding. On the dosing day, animals were singly housed and then returned to the paired cohort following the dosing/telemetry recording phase. Diet (Lab Diet® Certified Rodent Diet #5CR4) and tap water via an automatic water system were available ad libitum, except during designated procedures. Temperature and humidity were maintained according to the Testing Facility SOP. Animals were randomized into treatment groups using a standard, by weight, measured value randomization procedure. Body weight was determined for each rat prior to dosing.

The experimental design is summarized in Table 3:

TABLE 3

[symbol]

Experimental Design

| | | | | | | | Male |
|---|---|---|---|---|---|---|---|
| Group | | Study | Dose Level | Dose Level | Dose Conc. | Dose Volume | Animal |
| No. | Treatment^{a} | Day | (mg/kg/day) | (mg/kg/dose) | (mg/mL) | (mL/kg/dose) | Nos.^{b, c} |
| 1 | z | Day 1 | 0 | 0 | 0 | 1 | 1001, 1002, |
| | | | | | | | 2001, 2102, |
| | | Day 14/15 | 10 | 2.5 | 2.5 | | 3001, 3002, |
| | | | | | | | 4001, 4002, |
| | | Day 28/29 | 20 | 5 | 5 | | 5001, 5002, |
| | | | | | | | 6001, 6002 |
| 2 | | Day 1 | 0 | 0/1.25/2.5 | 0 | 1 | 1003, 1004, |
| | | | | | | | 2103, 2004, |
| | | Day 14/15 | 5 | 1.25 | 1.25 | | 3003, 3004, |
| | | | | | | | 4003, 4004, |
| | | Day 28/29 | 10 | 2.5 | 2.5 | | 5003, 5004, |
| | | | | | | | 6003, 6004 |
| 3 | | Day 1 | 0 | 0 | 0 | 1 | 1005, 1006, |
| | | | | | | | 2005, 2006, |
| | | Day 15 | 10 | 2.5 | 2.5 | | 3005, 3006, |
| | | | | | | | 4003, 4006, |
| | | Day 29 | 20 | 5 | 5 | | 5005, 5006, |
| | | | | | | | 6005, 6006 |

Conc. = Concentration
^{a}Each group received all 3 dose levels of the applicable test or control article in ascending order with a 1-week washout between dose levels. Each dose level was administered as 4 serial subcutaneous injections approximately 1 hour apart.
^{b}Neurobehavioral evaluations were conducted approximately 1 hour following the last cumulative dose of the series (Dose 4).
^{c}Hemodynamic endpoints, body temperature, and activity parameters were monitored continuously from at least 2 hours prior to Dose 1 to 7.5 hours following the last cumulative dose of each series (Dose 4). [symbol]
indicates data missing or illegible when filed

Treatment Group No. 1 received R-MDMA, No. 2 received S-MDMA, and No. 3 received racemic MDMA. As illustrated in the Table, a total of 36 rats were included in the study. The vehicle and 2 test article doses were administered in a volume of 1 mL/kg to each of 3 groups of n=12 telemeterized rats via subcutaneous injection four times on a given day, followed by neurobehavioral evaluations. This series was repeated 3 times on study. Each group received all 3 dose levels of the applicable test or control article in ascending order with a 1-week washout between dose levels.

Each dose level (expressed as mg (free base)/kg) was administered as 4 serial SC injections 1 hour apart, following a dosing protocol used by Biezonski et al. (2013) that reported significantly increased body temperature in animals that received 4 racemic MDMA SC injections at 10 mg/kg with an inter-dose interval of 1 h. Mortality/cage side clinical observations were conducted for all study animals at least twice daily beginning upon receipt through termination, except on days of receipt and termination where frequency was at least once daily. Detailed clinical observations were conducted for all study animals prior to each of the four cumulative doses in a series. Individual body weights were conducted for all study animals at transfer, prior to randomization, on the day of dosing on each of the 3 cycles of dosing. The body weights recorded at transfer and prior to randomization are not reported but are maintained in the study file. Veterinary care was available throughout the course of the study, and animals were examined by the veterinary staff as needed based on clinical signs or other changes. All animals were provided with HydroGel the night prior to their respective dosing days to potentially mitigate any animal welfare issues related to fluid loss/dehydration.

Neurobehavioral evaluations (blinded) were conducted for all study animals 1 hour following the last (4^{th}) cumulative dose of the series. The neurobehavioral parameters evaluated included: activity measurements (arousal/alertness, posture/body carriage, stereotypy, rearing, and appearance), autonomic measurements (exophthalmus, lacrimation, erected fur, pupil response, salivation, defecation, and palpebral closure/ptosis), excitability measurements (vocalizations, startle response, ease of removal, handling reactivity, and convulsions), neuromuscular measurements (gait/mobility, grip strength/response, air righting reflex, tremor, and body tone), physiological measurements (respiration and body temperature), and sensorimotor measurements (touch response/tactile reflex and thermal response).

Telemetry monitoring was conducted for all animals 2 hours prior to dosing and through to 7.5 hours following the last (4^{th}) cumulative dose of each series, which included continuous data collection of body temperature, activity, heart rate, and blood pressure (systolic, diastolic, and mean arterial) using telemetry.

Statistical analyses included within and between group comparisons as reflected in Table 4.

TABLE 4
Statistical Comparisons

Within
R-MDMA (0 mg/kg/day) × R-MDMA (10 mg/kg/day) × R-MDMA (20 mg/kg/day)
S-MDMA (0 mg/kg/day) × S-MDMA (5 mg/kg/day) × S-MDMA (10 mg/kg/day)
Rac.-MDMA (0 mg/kg/day) × Rac.-MDMA (10 mg/kg/day) × Rac.-MDMA (20 mg/kg/day)
Between
R-MDMA (0 mg/kg/day) × S-MDMA (0 mg/kg/day) × Rac.-MDMA (0 mg/kg/day)
R-MDMA (10 mg/kg/day) × S-MDMA (10 mg/kg/day) × Rac.-MDMA (10 mg/kg/day)
R-MDMA (10 mg/kg/day) × S-MDMA (10 mg/kg/day) × Rac.-MDMA (20 mg/kg/day)
R-MDMA (20 mg/kg/day) × Rac.-MDMA (20 mg/kg/day)
S-MDMA (5 mg/kg/day) × Rac.-MDMA (10 mg/kg/day)
R-MDMA (10 mg/kg/day) × R-MDMA (5 mg/kg/day) × Rac.-MDMA (10 mg/kg/day)
R-MDMA (20 mg/kg/day) × S-MDMA (10 mg/kg/day) × Rac.-MDMA (20 mg/kg/day)

The total period of time over which the statistical analyses were conducted encompassed the entire dosing period for all animals (i.e., Dose 1 through 7.5 hour post-4^{th} dose) in an effort to draw conclusions regarding the effects of the dosing procedure and cumulative changes by allowing comparisons between the cumulative dosing periods. The following statistical analyses were performed for each analysis segment.

TABLE 5
Statistical Model Analysis Procedure Segments
Statistical Mixed Model Analysis Procedure Segments

| Analysis | Hours Following | No. of | Minutes per |
|---|---|---|---|
| Segment | Start of Dosing | Intervals | Summary Interval |
| 1 | >0-1 | 6 | 10 |
| 2 | >1-2 | 6 | 10 |
| 3 | >2-3 | 6 | 10 |
| 4 | >3-4 | 6 | 10 |
| 5 | >4-10.5 | 39 | 10 |

All time-series data comparisons conducted via statistical analysis are relative to the control treatment and are based on the LSMean values. Additionally, the change values used for the statistical analysis were calculated using a baseline of the 2 hours of pre-dose data, prior to the first compound injection of the series. The raw change values were analyzed.

Results. Activity and arousal changes were observed across all groups (R-MDMA, S-MDMA, and racemic-MDMA) and were primarily dose-dependent, though the direction of change was dependent upon test article. R-MDMA-related changes included dose-dependent decreases in arousal/alertness and rearing, while S-MDMA and racemic-MDMA both exhibited a general increase in overall levels of arousal/alertness and rearing. The result observed for racemic MDMA administration was consistent with prior results that showed racemic MDMA induced hyperactivity in rodents (Gold and Koob, 1989; Kalivas et al., 1998; Doly et al., 2009). Regardless of the direction of behavioral change, all test articles increased incidence of unkempt appearance in a dose-dependent manner. Monoaminergic-related stereotypies (mouth movements/weaving, circling/retropulsion, sniffing) were observed in animals treated with S-MDMA and racemic-MDMA only at the highest tested doses (10 and 20 mg/kg/day, respectively). Those stereotypies have been reported for racemic-MDMA and compounds with comparable pharmacological mechanisms (Ellinwood Jr., 1980; Gauvin et al., 2019; Moscardo et al., 2007; Redfern et al., 2019; Willins and Meltzer, 1997).

Rearing (Lettfuss et al., 2013; Schenk and Bradbury, 2015) and the stereotypies observed in this study were previously demonstrated to be sensitive to acute racemic-MDMA administration. No stereotypical behaviors were reported in any R-MDMA-treated animal, and only one S-MDMA-treated animal was reported with stereotypical behaviors (repetitive circling); all other instances of stereotypical behavior (head weaving, circling, repetitive sniffing and rearing) were limited to the animals receiving 20 mg/kg/day racemic-MDMA. Table 6 summarizes the general rank order activity measurement effects.

TABLE 6
Rank Order Activity Measurement Effects

R-MDMA (0 mg/kg/day) < R-MDMA (10 mg/kg/day) < R-MDMA (20 mg/kg/day)
S-MDMA (0 mg/kg/day) < S-MDMA (5 mg/kg/day) < S-MDMA (10 mg/kg/day)
Rac-MDMA (0 mg/kg/day) < Rac-MDMA (20 mg/kg/day) < Rac-MDMA (10 mg/kg/day)
R-MDMA (0 mg/kg/day) = S-MDMA (0 mg/kg/day) = Rac-MDMA (0 mg/kg/day)
S-MDMA (10 mg/kg/day) = R-MDMA (10 mg/kg/day) = Rac-MDMA (10 mg/kg/day)
Rac.-MDMA (20 mg/kg/day) < S-MDMA (10 mg/kg/day) < R-MDMA (10 mg/kg/day)
Rac.-MDMA (20 mg/kg/day) < R-MDMA (20 mg/kg/day)
S-MDMA (5 mg/kg/day) < Rac.-MDMA (10 mg/kg/day)
S-MDMA (5 mg/kg/day) < R-MDMA (10 mg/kg/day) < Rac.-MDMA (10 mg/kg/day)
Rac.-MDMA (20 mg/kg/day) < S-MDMA (10 mg/kg/day) < R-MDMA (20 mg/kg/day)

Autonomic function changes were primarily limited to defecation, erected fur and salivation. These changes were observed across all groups (R-MDMA, S-MDMA, and racemic-MDMA), with a slightly higher incidence and severity occurring in racemic-MDMA-treated groups. Dose-dependent increased salivation and erected fur were the primary changes across groups. The observed autonomic effects of racemic-MDMA administration have been documented in the literature and linked to modulation of serotonergic systems (Frith et al., 1987; Haberzettl et al., 2013; Spanos and Yamamoto, 1989). Table 7 summarizes the general rank order autonomic measurement effects.

TABLE 7
Rank Order Autonomic Measurement Effects

R-MDMA (0 mg/kg/day) < R-MDMA (10 mg/kg/day) < R-MDMA (20 mg/kg/day)
S-MDMA (0 mg/kg/day) < S-MDMA (5 mg/kg/day) < S-MDMA (10 mg/kg/day)
Rac-MDMA (0 mg/kg/day) < Rac-MDMA (10 mg/kg/day) < Rac-MDMA (20 mg/kg/day)
R-MDMA (0 mg/kg/day) = S-MDMA (0 mg/kg/day) = Rac-MDMA (0 mg/kg/day)
R-MDMA (10 mg/kg/day) < Rac-MDMA (10 mg/kg/day) < S-MDMA (10 mg/kg/day)
R-MDMA (10 mg/kg/day) < S-MDMA (10 mg/kg/day) = Rac-MDMA (20 mg/kg/day)
R-MDMA (20 mg/kg/day) < Rac-MDMA (20 mg/kg/day)
S-MDMA (5 mg/kg/day) < Rac.-MDMA (10 mg/kg/day)
R-MDMA (10 mg/kg/day) < S-MDMA (5 mg/kg/day) < Rac.-MDMA (10 mg/kg/day)
R-MDMA (20 mg/kg/day) < S-MDMA (10 mg/kg/day) < Rac-MDMA (20 mg/kg/day)

Excitability measurements were affected across all groups. The most notable changes in excitability were observed on ease of removal from the home cage and handling reactivity measurements, which is a common finding for psychomotor stimulants. Dose-dependent increased difficulty in removal from the home cage and increased handling reactivity was observed across all groups (R-MDMA, S-MDMA, and racemic-MDMA). Table 8 summarizes the general rank order excitability measurement effects.

TABLE 8
Rank Order Excitability Measurement Effects

R-MDMA (0 mg/kg/day) < R-MDMA (10 mg/kg/day) < R-MDMA (20 mg/kg/day)
S-MDMA (0 mg/kg/day) < S-MDMA (5 mg/kg/day) < S-MDMA (10 mg/kg/day)
Rac-MDMA (0 mg/kg/day) < Rac-MDMA (10 mg/kg/day) < Rac-MDMA (20 mg/kg/day)
R-MDMA (0 mg/kg/day) = S-MDMA (0 mg/kg/day) = Rac-MDMA (0 mg/kg/day)
R-MDMA (10 mg/kg/day) < S-MDMA (10 mg/kg/day) = Rac-MDMA (10 mg/kg/day)
R-MDMA (10 mg/kg/day) < S-MDMA (10 mg/kg/day) < Rac-MDMA (20 mg/kg/day)
R-MDMA (20 mg/kg/day) < Rac-MDMA (20 mg/kg/day)
S-MDMA (5 mg/kg/day) < Rac-MDMA (10 mg/kg/day)
R-MDMA (10 mg/kg/day) < S-MDMA (5 mg/kg/day) < Rac.-MDMA (10 mg/kg/day)
R-MDMA (20 mg/kg/day) = S-MDMA (10 mg/kg/day) < Rac-MDMA (20 mg/kg/day)

Neuromuscular function changes across treatment conditions were primarily limited to increased body tone, gait/mobility impairments, and grip strength deficits. General gait/mobility changes were only observed in the S-MDMA- and racemic-MDMA-treated groups, though dose-dependent statistically significant decreases in forelimb grip strength were observed across all test articles (R-MDMA, S-MDMA, and racemic-MDMA). Dose-dependent racemic-MDMA-related changes also included observations of ataxia, hunched posture, and tip-toe walking, and were primarily noted in the 20 mg/kg/day racemic-MDMA-treated animals. Table 9 summarizes the general rank order neuromuscular measurement effects.

TABLE 9
Rank Order Neuromuscular Measurement Effects

R-MDMA (0 mg/kg/day) < R-MDMA (10 mg/kg/day) < R-MDMA (20 mg/kg/day)
S-MDMA (0 mg/kg/day) < S-MDMA (5 mg/kg/day) = S-MDMA (10 mg/kg/day)
Rac-MDMA (0 mg/kg/day) < Rac-MDMA (10 mg/kg/day) < Rac-MDMA (20 mg/kg/day)
R-MDMA (0 mg/kg/day) = S-MDMA (0 mg/kg/day) = Rac-MDMA (0 mg/kg/day)
R-MDMA (10 mg/kg/day) < S-MDMA (10 mg/kg/day) < Rac-MDMA (10 mg/kg/day)
R-MDMA (10 mg/kg/day) < S-MDMA (10 mg/kg/day) < Rac-MDMA (20 mg/kg/day)
R-MDMA (20 mg/kg/day) < Rac-MDMA (20 mg/kg/day)
S-MDMA (5 mg/kg/day) < Rac.-MDMA (10 mg/kg/day)
R-MDMA (10 mg/kg/day) < S-MDMA (5 mg/kg/day) < Rac.-MDMA (10 mg/kg/day)
R-MDMA (20 mg/kg/day) = S-MDMA (10 mg/kg/day) < Rac-MDMA (20 mg/kg/day)

Physiological changes as measured in the neurobehavioral evaluation included mixed effects on respiration and increased body temperature. Rapid respiration was noted in the S- and racemic-MDMA treated animals. Rapid respiration could be attributed to exacerbating the overall increased stress level induced by the FOB evaluation, in conjunction with the known pharmacology of MDMA and the role of serotonin in respiratory function in a number of species (Hilaire et al., 2010). Body temperature increases are one of the hallmark observations following racemic MDMA administration and have been widely reported in both the nonclinical (Banks et al. 2007; Berquist et al., 2020; Bexis and Docherty, 2006; Malberg and Seiden, 1998; Malpass et al., 1999; Wright et al., 2012) and clinical literature (Kalant, 2001; Liechti, 2014; Parrott and Young, 2014). Table 10 summarizes the general rank order physiological measurement effects.

TABLE 10
Rank Order Physiological Measurement Effects

R-MDMA (0 mg/kg/day) = R-MDMA (10 mg/kg/day) = R-MDMA (20 mg/kg/day)
S-MDMA (0 mg/kg/day) < S-MDMA (5 mg/kg/day) < S-MDMA (10 mg/kg/day)
Rac-MDMA (0 mg/kg/day) < Rac-MDMA (10 mg/kg/day) < Rac-MDMA (20 mg/kg/day)
R-MDMA (0 mg/kg/day) = S-MDMA (0 mg/kg/day) = Rac-MDMA (0 mg/kg/day)
R-MDMA (10 mg/kg/day) < S-MDMA (10 mg/kg/day) < Rac-MDMA (10 mg/kg/day)
R-MDMA (10 mg/kg/day) < S-MDMA (10 mg/kg/day) = Rac-MDMA (20 mg/kg/day)
R-MDMA (20 mg/kg/day) < Rac-MDMA (20 mg/kg/day)
S-MDMA (5 mg/kg/day) < Rac.-MDMA (10 mg/kg/day)
R-MDMA (10 mg/kg/day) < S-MDMA (5 mg/kg/day) < Rac.-MDMA (10 mg/kg/day)
R-MDMA (20 mg/kg/day) < S-MDMA (10 mg/kg/day) = Rac-MDMA (20 mg/kg/day)

Sensorimotor function changes included effects on touch/tactile reflex and increased thermal response times. Statistically significant increases in thermal response times were observed across all groups (R-MDMA, S-MDMA, and racemic-MDMA) and at levels at least two-fold that observed in the vehicle-treated groups. Increased latency to escape a painful stimulus (hot plate analgesiometer) has been noted previously in rats acutely treated with racemic MDMA (Crisp et al., 1989). Table 11 summarizes the general rank order sensorimotor measurement effects.

TABLE 11
Rank Order Sensorimotor Measurement Effects

R-MDMA (0 mg/kg/day) < R-MDMA (10 mg/kg/day) < R-MDMA (20 mg/kg/day)
S-MDMA (0 mg/kg/day) < S-MDMA (10 mg/kg/day) < S-MDMA (5 mg/kg/day)
Rac-MDMA (0 mg/kg/day) < Rac-MDMA (10 mg/kg/day) < Rac-MDMA (20 mg/kg/day)
R-MDMA (0 mg/kg/day) = S-MDMA (0 mg/kg/day) = Rac-MDMA (0 mg/kg/day)
S-MDMA (10 mg/kg/day) < Rac-MDMA (10 mg/kg/day) < R-MDMA (10 mg/kg/day)
S-MDMA (10 mg/kg/day) < Rac-MDMA (20 mg/kg/day) < R-MDMA (20 mg/kg/day)
Rac-MDMA (20 mg/kg/day) < R-MDMA (20 mg/kg/day)
Rac-MDMA (10 mg/kg/day) < S-MDMA (5 mg/kg/day)
S-MDMA (5 mg/kg/day) < Rac-MDMA (10 mg/kg/day) < R-MDMA (10 mg/kg/day)
S-MDMA (10 mg/kg/day) < Rac-MDMA (20 mg/kg/day) < R-MDMA (20 mg/kg/day)

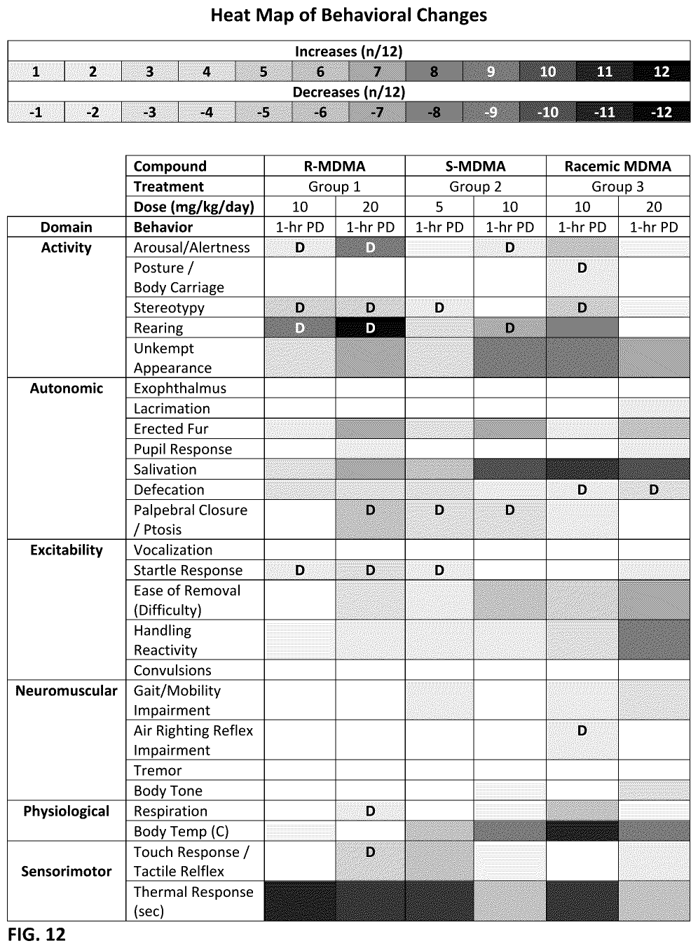

A newer analysis method of multidimensional data from FOB evaluations has been proposed (Mathiasen and Moser, 2018). This method generates a “heat map” and provides a visual summary of the relationship between compound, dose, and major impairments. FIG. 12 depicts a heat map of the behavioral effects observed on this study and represents the net behavioral changes observed for each compound (R-MDMA, S-MDMA, or racemic-MDMA) and each dose level. Behavior is represented in six separate domains. Unmarked, darkened gradations denote net increases (n/12), and darkened gradations identified with the letter “D” denote net decreases (n/12) for a given behavior relative to observed effects of vehicle administration; the darker the gradation, the greater the number of net increases or decreases observed. For clarity, any unshaded (i.e., white) boxes in FIG. 12 indicate no change in occurrence.

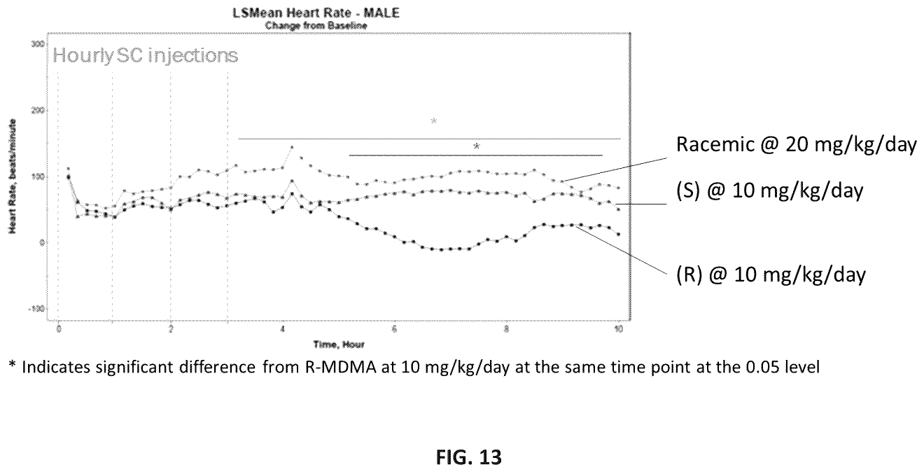

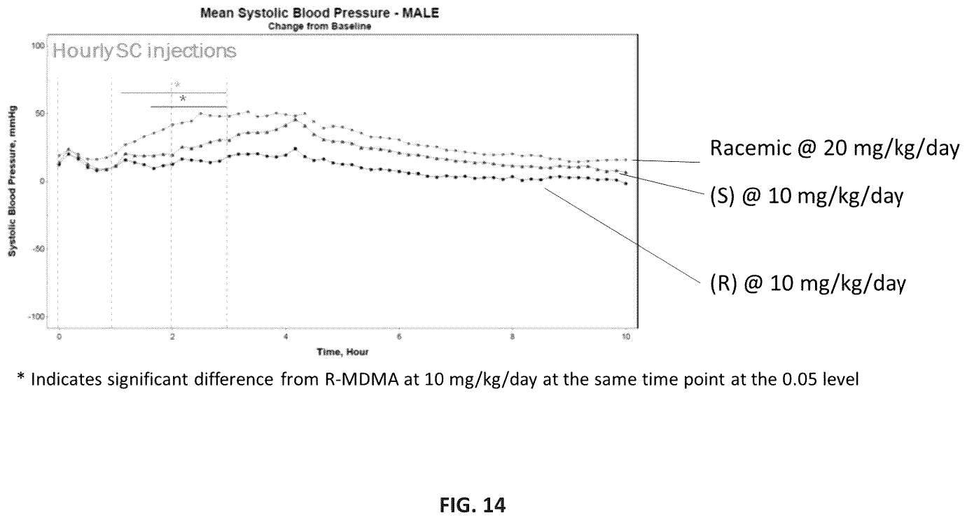

Beyond the FOB evaluations, dose-dependent blood pressure-increasing effects were observed for every hemodynamic parameter (systolic/diastolic/mean arterial blood pressures and heart rate) and each test article (R-MDMA, S-MDMA, and racemic-MDMA), and cumulative doses evaluated (5, 10, or 20 mg/kg/day). Although all test articles produced blood pressure effects, R-MDMA produced the least pronounced effects regarding overall magnitude of change and was the only test article examined which produced recoverable effects by the end of telemetry monitoring. R-MDMA-related changes included heart rate (up to +23%) and blood pressure increases (up to +28% [mean arterial pressure]). S-MDMA produced the next greatest heart rate (up to +28%) and blood pressure effects (up to +36% [mean arterial pressure]), while racemic-MDMA produced the greatest increases in both heart rate (up to +37%) and blood pressures (up to +46% [mean arterial pressure]). Although doses were administered repeatedly throughout the telemetry monitoring period, no stepwise increases in blood pressure or heart rate were observed in R-MDMA-treated groups, as was observed in the S-MDMA- and racemic-MDMA-treated groups. R-MDMA and S-MDMA were comparable in the direction, magnitude, and duration of change in heart rate, though the doses of S-MDMA evaluated (5 and 10 mg/kg/day) were half that of R-MDMA (10 and 20 mg/kg/day), indicative of increased potency of the S- relative to the R-enantiomer to affect heart rate. FIGS. 13 and 14 illustrate the heart rate and mean arterial pressure effects (respectively) of enantiomeric-equivalent dosing conditions (i.e., 10 mg/kg/day R-MDMA, 10 mg/kg/day S-MDMA and 20 mg/kg/day racemic MDMA). As FIG. 13 shows, racemic MDMA (20 mg/kg/day) exhibited significantly increased heart rate compared to R-MDMA (10 mg/kg/day) from approximately 3-10 hours post initiation of dosing, while S-MDMA (10 mg/kg/day) exhibited significantly increased heart rate compared to R-MDMA (10 mg/kg/day) from approximately 5-10 hours post initiation of dosing. FIG. 14 shows that racemic MDMA (20 mg/kg/day) exhibited significantly increased systolic blood pressure compared to R-MDMA (10 mg/kg/day) from approximately 1-3 hours post initiation of dosing, while S-MDMA (10 mg/kg/day) exhibited significantly increased systolic blood pressure compared to R-MDMA (10 mg/kg/day) from approximately 1.5-3 hours post initiation of dosing.

Tables 12 and 13 summarize the general rank order heart rate effects and blood pressure effects, respectively.

TABLE 12
Rank Order Heart Rate Measurement Effects

R-MDMA (0 mg/kg/day) < R-MDMA (10 mg/kg/day) < R-MDMA (20 mg/kg/day)
S-MDMA (0 mg/kg/day) < S-MDMA (5 mg/kg/day) < S-MDMA (10 mg/kg/day)
Rac-MDMA (0 mg/kg/day) < Rac-MDMA (10 mg/kg/day) < Rac-MDMA (20 mg/kg/day)
R-MDMA (0 mg/kg/day) = S-MDMA (0 mg/kg/day) = Rac-MDMA (0 mg/kg/day)
R-MDMA (10 mg/kg/day) < S-MDMA (10 mg/kg/day) < Rac-MDMA (10 mg/kg/day)
R-MDMA (10 mg/kg/day) < S-MDMA (10 mg/kg/day) < Rac-MDMA (20 mg/kg/day)
R-MDMA (20 mg/kg/day) < Rac-MDMA (20 mg/kg/day)
S-MDMA (5 mg/kg/day) < Rac.-MDMA (10 mg/kg/day)
R-MDMA (10 mg/kg/day) < S-MDMA (5 mg/kg/day) < Rac.-MDMA (10 mg/kg/day)
R-MDMA (20 mg/kg/day) < S-MDMA (10 mg/kg/day) < Rac-MDMA (20 mg/kg/day)

TABLE 13
Rank Order Blood Pressures (Systolic, Diastolic, and Mean Arterial) Effects

R-MDMA (0 mg/kg/day) < R-MDMA (10 mg/kg/day) < R-MDMA (20 mg/kg/day)
S-MDMA (0 mg/kg/day) < S-MDMA (5 mg/kg/day) < S-MDMA (10 mg/kg/day)
Rac-MDMA (0 mg/kg/day) < Rac-MDMA (10 mg/kg/day) < Rac-MDMA (20 mg/kg/day)
R-MDMA (0 mg/kg/day) = S-MDMA (0 mg/kg/day) = Rac-MDMA (0 mg/kg/day)
R-MDMA (10 mg/kg/day) < S-MDMA (10 mg/kg/day) < Rac-MDMA (10 mg/kg/day)
R-MDMA (10 mg/kg/day) < S-MDMA (10 mg/kg/day) < Rac-MDMA (20 mg/kg/day)
R-MDMA (20 mg/kg/day) < Rac-MDMA (20 mg/kg/day)
S-MDMA (5 mg/kg/day) < Rac.-MDMA (10 mg/kg/day)
R-MDMA (10 mg/kg/day) < S-MDMA (5 mg/kg/day) < Rac.-MDMA (10 mg/kg/day)
R-MDMA (20 mg/kg/day) < S-MDMA (10 mg/kg/day) < Rac-MDMA (20 mg/kg/day)

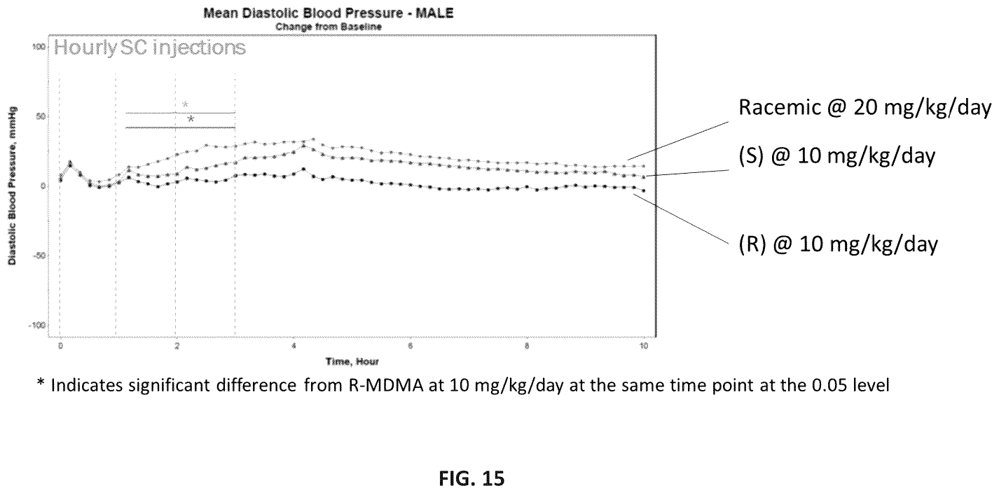

FIG. 15 illustrates the effects of enantiomeric-equivalent dosing conditions of R-MDMA, S-MDMA and racemic MDMA on diastolic blood pressure change from baseline over time post initiation of dosing. Racemic MDMA (20 mg/kg/day) and S-MDMA (10 mg/kg/day) exhibited significantly increased diastolic blood pressure compared to R-MDMA (10 mg/kg/day) from approximately 1-3 hours post initiation of dosing.

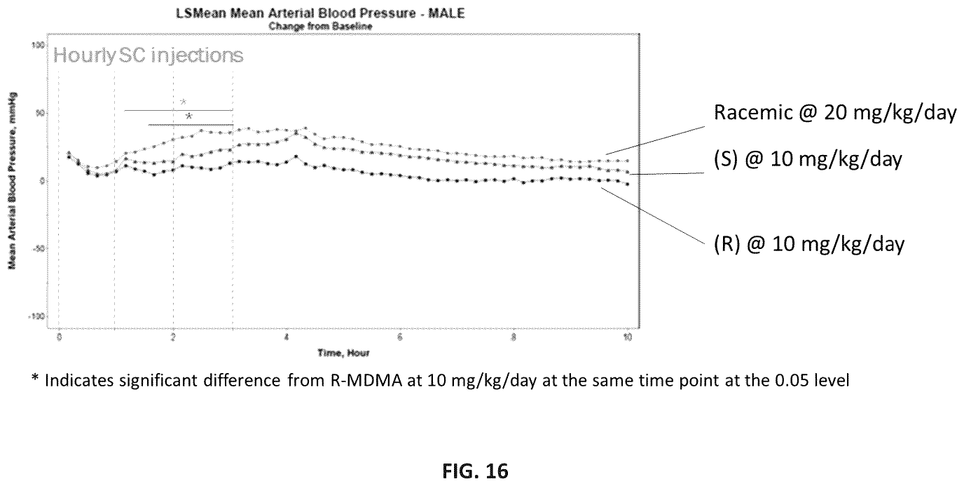

FIG. 16 illustrates the effects of enantiomeric-equivalent dosing conditions of R-MDMA, S-MDMA and racemic MDMA on mean arterial blood pressure change from baseline over time post initiation of dosing. Racemic MDMA (20 mg/kg/day) exhibited significantly increased mean arterial blood pressure compared to R-MDMA (10 mg/kg/day) from approximately 1-3 hours post initiation of dosing, while S-MDMA (10 mg/kg/day) exhibited significantly increased mean arterial blood pressure compared to R-MDMA (10 mg/kg/day) from approximately 1.5-3 hours post initiation of dosing.

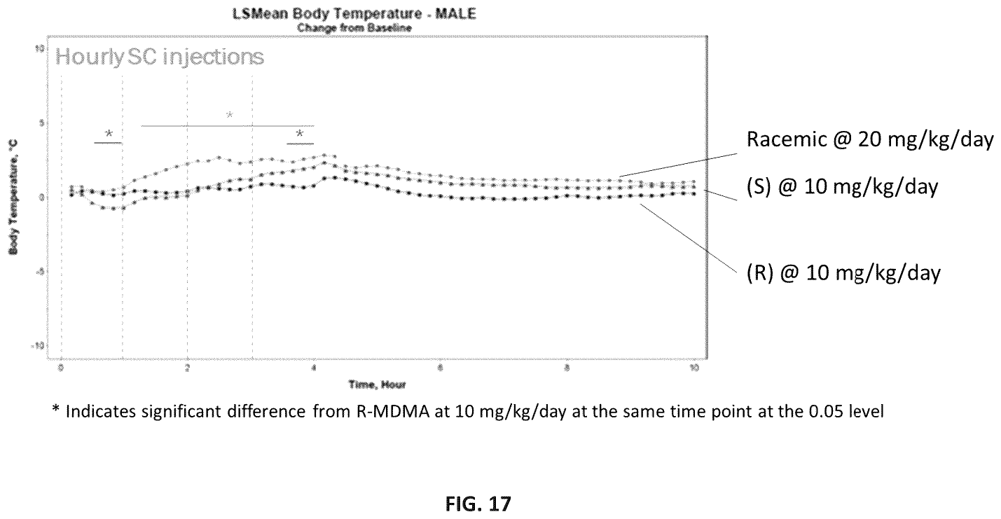

Test article-related body temperature effects were observed at every cumulative dose level (5, 10, or 20 mg/kg/day) and with every compound (R-MDMA, S-MDMA, or racemic-MDMA). In general, S-MDMA and racemic-MDMA produced a hyperthermic response whereas R-MDMA-related effects were dose-dependent-10 mg/kg/day produced a hypothermic response, and 20 mg/kg/day produced a hyperthermic response. The observed body temperature effects are generally consistent with the literature surrounding MDMA administration and its effects on thermoregulatory processes. Racemic-MDMA administration has been shown to produce bidirectional (i.e., hypo/hyperthermia) effects dependent upon environmental variables such as ambient temperature by influencing thermoregulatory processes (Dafters, 1994; Green et al., 2003, 2004; Malberg and Seiden, 1998; Wright et al., 2012). R-MDMA has also been shown to decrease body temperature in mice relative to vehicle, whereas racemic-MDMA produced statistically significant increases in body temperature at a 2.5-fold lower dose (Curry et al., 2018). A report by Biezonski et al., 2013, showed a significant increase in body temperature in rats following cumulative SC administration of 4 hourly racemic MDMA injections at 10 mg/kg/injection, consistent with the current study findings following cumulative SC administration of racemic MDMA at 5 mg/kg/injection. Although all test articles produced body temperature effects, R-MDMA produced the least severe effects regarding overall mean magnitude of change (peak body temperatures: R-MDMA=38.6° C.; S-MDMA=39.5° C.; racemic-MDMA=40.0° C.). FIG. 17 illustrates the body temperature effects of enantiomeric-equivalent dosing conditions (i.e., 10 mg/kg/day R-MDMA, 10 mg/kg/day S-MDMA and 20 mg/kg/day racemic MDMA). As shown, racemic MDMA (20 mg/kg/day) exhibited significantly increased body temperature compared to R-MDMA (10 mg/kg/day) from approximately 1-4 hours post initiation of dosing, while S-MDMA (10 mg/kg/day) significantly decreased body temperature compared to R-MDMA (10 mg/kg/day) from approximately 0.5-1 hours post initiation of dosing and increased body temperature compared to R-MDMA (10 mg/kg/day) from approximately 3.5-4 hours post initiation of dosing. Table 14 summarizes the general rank order body temperature effects.

TABLE 14
Rank Order Body Temperature Effects

R-MDMA (0 mg/kg/day) < R-MDMA (10 mg/kg/day) < R-MDMA (20 mg/kg/day)
S-MDMA (0 mg/kg/day) < S-MDMA (5 mg/kg/day) < S-MDMA (10 mg/kg/day)
Rac-MDMA (0 mg/kg/day) < Rac-MDMA (10 mg/kg/day) < Rac-MDMA (20 mg/kg/day)
R-MDMA (0 mg/kg/day) = S-MDMA (0 mg/kg/day) = Rac-MDMA (0 mg/kg/day)
R-MDMA (10 mg/kg/day) < S-MDMA (10 mg/kg/day) < Rac-MDMA (10 mg/kg/day)
R-MDMA (10 mg/kg/day) < S-MDMA (10 mg/kg/day) < Rac-MDMA (20 mg/kg/day)
R-MDMA (20 mg/kg/day) < Rac-MDMA (20 mg/kg/day)
S-MDMA (5 mg/kg/day) < Rac.-MDMA (10 mg/kg/day)
R-MDMA (10 mg/kg/day) < S-MDMA (5 mg/kg/day) < Rac.-MDMA (10 mg/kg/day)
R-MDMA (20 mg/kg/day) < S-MDMA (10 mg/kg/day) < Rac-MDMA (20 mg/kg/day)

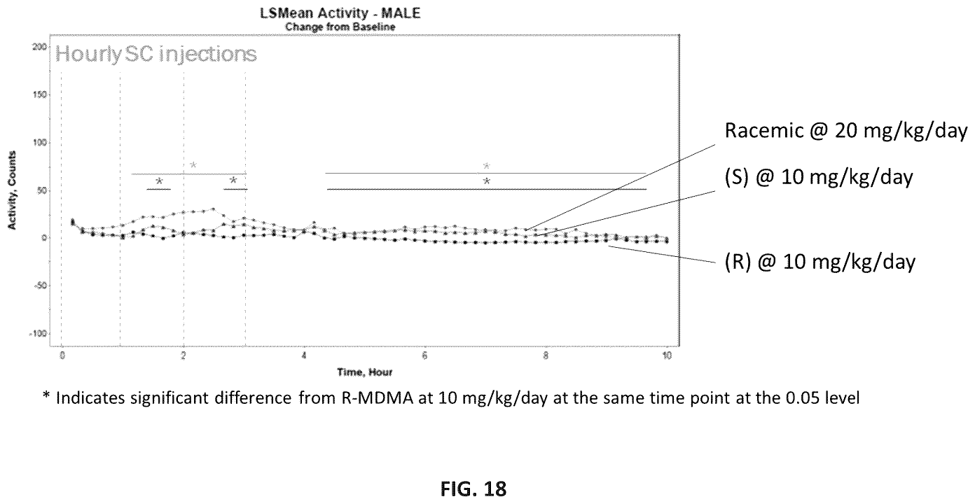

Activity-increasing effects (as measured via implanted telemetry devices) were observed across all test articles (R-MDMA, S-MDMA, and racemic-MDMA) and at all cumulative doses (5, 10, or 20 mg/kg/day). The most substantial effects were observed in the S-MDMA and racemic-MDMA-treated animals and were comparable across equivalent cumulative doses. FIG. 18 shows the activity effects of enantiomeric-equivalent dosing conditions (i.e., 10 mg/kg/day R-MDMA, 10 mg/kg/day S-MDMA and 20 mg/kg/day racemic MDMA). As shown, racemic MDMA (20 mg/kg/day) exhibited significantly increased activity compared to R-MDMA (10 mg/kg/day) from approximately 1-3 and 4.5-10 hours post initiation of dosing, while S-MDMA (10 mg/kg/day) significantly increased activity compared to R-MDMA (10 mg/kg/day) from approximately 1.5-2, 2.5-3 and 4.5-10 hours post initiation of dosing. Table 15 summarizes the general rank order general activity effects.

TABLE 15
Rank Order General Activity Effects

R-MDMA (0 mg/kg/day) < R-MDMA (10 mg/kg/day) < R-MDMA (20 mg/kg/day)
S-MDMA (0 mg/kg/day) < S-MDMA (5 mg/kg/day) < S-MDMA (10 mg/kg/day)
Rac-MDMA (0 mg/kg/day) < Rac-MDMA (10 mg/kg/day) < Rac-MDMA (20 mg/kg/day)
R-MDMA (0 mg/kg/day) = S-MDMA (0 mg/kg/day) = Rac-MDMA (0 mg/kg/day)
R-MDMA (10 mg/kg/day) < S-MDMA (10 mg/kg/day) < Rac-MDMA (10 mg/kg/day)
R-MDMA (10 mg/kg/day) < S-MDMA (10 mg/kg/day) < Rac-MDMA (20 mg/kg/day)
R-MDMA (20 mg/kg/day) < Rac-MDMA (20 mg/kg/day)
S-MDMA (5 mg/kg/day) < Rac.-MDMA (10 mg/kg/day)
R-MDMA (10 mg/kg/day) < S-MDMA (5 mg/kg/day) < Rac.-MDMA (10 mg/kg/day)
R-MDMA (20 mg/kg/day) < S-MDMA (10 mg/kg/day) < Rac-MDMA (20 mg/kg/day)

Conclusions: All test articles (R-MDMA, S-MDMA, and racemic-MDMA) affected neurobehavioral and physiological function as evaluated within this study. At the group level, the neurobehavioral domains most affected by R-MDMA administration (10 or 20 mg/kg/day) were activity, autonomic, excitability, and sensorimotor. All domains were affected by S-MDMA (5 or 10 mg/kg/day) and racemic-MDMA administration (10 or 20 mg/kg/day). S-MDMA and racemic-MDMA elicited a greater incidence and severity of behavioral and physiological changes than R-MDMA under the noted conditions of the present study.

### Example 7

### Acute Pharmocokinetic and Persistent Brain Monoamine Concentration Effects of Racemic, S(+)- and R(−)-MDMA in Rats

This example evaluates the acute pharmacokinetic and persistent brain monoamine concentration effects of the test articles, R-MDMA, S-MDMA and racemic-MDMA, following cumulative SC injections administered 4 times on a single dosing day (with serial injections 1 hour apart for each animal), in adult male CD® (Sprague Dawley) rats (Charles River Laboratories). The study design was as described below and summarized in the experimental design Tables A-B, with Test Articles Groups 1-3 (R-MDMA^{a,b}); 4-6 (S-MDMA^{a,b,c}); 7-9 (Rac-MDMA^{a,b}).

TABLE A
Experimental Design

| | | Dose | | | | | |
|---|---|---|---|---|---|---|---|
| Group | Test | Level | Week | Week | Week | Week | No. of |
| No. | Article | (mg/kg) | 1 | 2 | 3 | 4 | Animals |
| 1 | z | 0 | 2 | 2 | 2 | 2 | 8 |
| 2 | | 5/2.5 | 2 | 2 | 2 | 2 | 8 |
| 3 | | 10/5 | 2 | 2 | 2 | 2 | 8 |
| 4 | | 0 | 2 | 2 | 2 | 2 | 8 |
| 5 | | 5/2.5/1.25 | 2 | 2 | 2 | 2 | 8 |
| 6 | | 10/5/2.5 | 2 | 2 | 2 | 2 | 8 |
| 7 | | 0 | 2 | 2 | 2 | 2 | 8 |
| 8 | | 5/2.5 | 2 | 2 | 2 | 2 | 8 |
| 9 | | 10/5 | 2 | 2 | 2 | 2 | 8 |

^{a} All doses expressed on a mg (free base)/kg/injection basis; total targeted doses were 0, 20, and 40 mk/kg/day by 4 serial subcutaneous injections on the dosing day. Injection volumes were 1 mL/kg. Dose formulation concentrations were 0, 5, and 10 mg (free base)/mL.
^{b} Due to observed mortality following the first week, the subsequent dosing weeks’ (weeks 2-4) total targeted doses were 0, 10, and 20 mg/kg/day by 4 serial subcutaneous injections on the dosing day. Injection volumes were 1 mL/kg. Dose formulation concentrations were 0, 2.5, and 5 mg (free base)/mL.
^{c} Due to observed morbidity following the second week, the subsequent dosing weeks’ (weeks 3-4) total targeted doses were 0, 5, and 10 mg/kg/day by 4 serial subcutaneous injections on the dosing day. Injection volumes were 1 mL/kg. Dose formulation concentrations were 0, 1.25, 2.5 mg (free base)/mL.

TABLE B
Groups 10-13 (R-MDMA); 14-18 (S-MDMA); 19-22 (Rac-MDMA)
Actual Dose Comparison Experimental Design
For better comparison purposes, the animals were redistributed
from Groups 1-9 into Groups 10-22 to align them by actual
dose level received due to mortality noted on study.

| Group | Test | Dose | Total No. | Original |
|---|---|---|---|---|
| Number | Material | (mg/kg) | of Animals | Animal Numbers |
| 10 | | 0 | 8 | 1001-1008 |
| 11 | | 2.5 | 6 | 2003-2008 |
| 12 | | 5 | 8 | 2001-2002, 3003-3008 |
| 13 | | 10 | 2 | 3001-3002 |
| 14 | | 0 | 8 | 4001-4008 |
| 15 | | 1.25 | 4 | 5005-5008 |
| 16 | | 2.5 | 6 | 5003-5004, 6005-6008 |
| 17 | | 5 | 4 | 5001-5002, 6003-6004 |
| 18 | | 10 | 2 | 6001-6002 |
| 19 | | 0 | 8 | 7001-7008 |
| 20 | | 2.5 | 6 | 8003-8008 |
| 21 | | 5 | 8 | 8001-8002, 9003-9008 |
| 22 | | 10 | 2 | 9001-9002 |

Methods. Adult male CD® (Sprague Dawley) rats (Charles River Laboratories) were acclimated to the laboratory environment for at least 1 week prior to experimentation. Animals were pair-housed in solid bottom cages with nonaromatic bedding. On the dosing day, animals were singly housed and then returned to the paired cohort following the dosing/pharmacokinetic sampling phase. Diet (Lab Diet® Certified Rodent Diet #5CR4) and tap water via an automatic water system were available ad libitum, except during designated procedures. Temperature and humidity were maintained according to the Testing Facility SOP. Animals were randomized into treatment groups using a standard, by weight, measured value randomization procedure. Body weight was determined for each rat prior to dosing.

Two animals from each treatment group were dosed each week over 4 weeks, in an effort to align the inlife phase with the multi-week inlife phase of the companion telemeterized rat study using the same test articles, doses and cumulative SC dosing protocol. On the day of dosing, test article or vehicle was administered SC in the scapular region on the back four times, with serial injections 1 hour apart (or closest possible approximation thereof), for each animal, following the same dosing protocol used in the rat FOB/telemetry study and reported by Biezonski et al. (2013) to significantly reduce frontal cortical 5-HT and 5-HIAA and striatal DOPAC and HVA concentrations in rats 1 week following cumulative SC administration of 4 hourly racemic MDMA injections at 10 mg/kg/injection. Whole blood (0.3 mL) was collected from the sublingual or other suitable vein into K2EDTA coated tubes at multiple timepoints (i.e., 0.5 h prior to the 4^{th} injection and 0.5, 1, 2, 4, 6, 8 and 24 h after the 4^{th} injection; time points equivalent to 2.5, 3.5, 4, 5, 7, 9, 11 and 27 h after the 1^{st} injection) from 2 animals bled at alternating time points. Whole blood was stored on wet ice until centrifuged within 30 minutes of collection for processing and plasma was stored frozen (−60 to −90° C.).

Samples from animals dosed with racemic MDMA were analyzed for both R- and S-MDMA as well as R- and S-MDA. Samples from animals dosed with R-MDMA were analyzed for R-MDMA and R-MDA, and samples from animals dosed with S-MDMA were analyzed for S-MDMA and S-MDA using a method qualified under Testing Facility Study No. 3416-011. Racemic MDMA and Racemic MDA concentrations were calculated by adding together the R and S enantiomers. A non-compartmental approach consistent with the SC route of administration was used for pharmacokinetic parameter estimation. For the purposes of kinetic analysis, all data were interpreted from all animals being dosed on the same day (Day 1). The mean, standard deviation (SD), and coefficient of variation (CV) were calculated for R-MDMA, S-MDMA, total MDMA, R-MDA, S-MDA, and total MDA plasma concentrations at each time point. Concentrations less than the lower limit of quantitation (LLOQ=10.0 ng/mL for R-MDMA, S-MDMA, R-MDA, and S-MDA) were set to 0 for pharmacokinetic analysis. Composite plasma concentration-time profiles were constructed for each respective analyte and dose level from which pharmacokinetic parameters were derived.

The mean R-MDMA, S-MDMA, total MDMA, R-MDA, S-MDA, and total MDA plasma concentration-time profiles from R-MDMA, S-MDMA, and Racemic-MDMA-treated animals were analyzed using model-independent methods (Gibaldi, 1982). For each dose group, the following pharmacokinetic parameters were determined: maximum observed plasma concentration (Cₘₐₓ), time of maximum observed plasma concentration (Tₘₐₓ), and area under the plasma concentration-time curve (AUC). The AUC from time 0 to 27 hours (AUC_{0-27 hr}), and the AUC from time 0 to the time of the final quantifiable sample (AUC_{Tlast}) were calculated by the linear trapezoidal method for all dose groups with at least 3 consecutive quantifiable concentrations. Zero (0) was used as an estimate of the 0-hour (predose) concentration. Half-life values (T_{1/2}) were reported for composite plasma concentration-time profiles with sufficient plasma concentrations in the terminal elimination phase (at least 3 samples not including Tₘₐₓ) and an adjusted R² of ≥0.9. The metabolite to parent ratios (M:P) were calculated for each dose group using the following formulas:

M:P=AUC_{0-27 hr R-MDA}÷AUC_{0-27 hr R-MDMA}

M:P=AUC_{0-27 hr S-MDA}÷AUC_{0-27 hr S-MDMA}

M:P=AUC_{0-27 hr Total MDA}÷AUC_{0-27 hr Total MDMA}

When Tₗₐₛₜ did not equal the last collection interval, the percent of AUC extrapolated (% AUCExtrap) for AUC_{0-27 hr} was calculated as:

% AUCExtrap=[(AUC_{0-27 hr}−AUC_{Tlast})/AUC_{0-27 hr}]×100

All AUC_{0-27 hr} values were calculated and were not >25% extrapolation. The % AUCExtrap data are not reported but are maintained in the study file. The 40 mg/kg/day dose level was excluded for all analytes, due to having limited data (N=1/timepoint for the majority of timepoints).

One week following cumulative dosing, each animal was sacrificed following carbon dioxide inhalation using a rodent guillotine. The whole brain was removed, and 50 mg samples were collected, one from the frontal cortex and one from the striatum, from each animal. Samples were rinsed with PBS, dried on a wypall, weighed and flash frozen in liquid nitrogen and stored frozen (−60 to −90° C.). Frontal cortex samples were analyzed for serotonin (5-HT) and its metabolite 5-hydroxyindole-3-acetic acid (5-HIAA) and striatum samples were analyzed for dopamine (DA) and its metabolites 3,4-dihydroxyphenylacetic acid (DOPAC) and homovanillic acid (HVA) using methods qualified under Testing Facility Study No. 3416-022 (5-HT, 5-HIAA, and DA) and Testing Facility Study No. 3416-023 (DOPAC and HVA). Water with 0.1% formic acid was used as the surrogate calibrant matrix for the quantitation of brain tissue samples. A two-way analysis of variance was conducted using test article, dose level and the interaction of test article and dose level as effects for the model. If the interaction effect of test article and dose level was significant (p<0.05), then linear contrasts were constructed for all pair-wise comparisons and reported at the 0.05 and 0.01 significance levels after adjustment for multiple comparisons using the methods of Edwards and Berry (1987). All tests were two-tailed unless indicated otherwise.

### Results.

Plasma: The results from calibration standards and quality control samples demonstrated acceptable performance of the method for all reported concentrations. Tables 16-21 below summarize the plasma pharmacokinetic parameters for R-MDMA, S-MDMA, total MDMA, R-MDA, S-MDA and total MDA following cumulative SC administration of R-MDMA, S-MDMA or racemic MDMA.

TABLE 16
R-MDMA Pharmacokinetic Parameters Following QID SC inj. of 2.5 and 5 mg/kg/dose
(total daily doses of 10 and 20 mg/kg/day) R-MDMA to Male Rats.

| | Dose | C [symbol] | C [symbol] /Dose | T [symbol] | T [symbol] | AUC [symbol] | AUC [symbol] | AUC [symbol] /Dose | T_{1/2} |
|---|---|---|---|---|---|---|---|---|---|
| Analyte | (mg/kg/day) | (ng/mL) | (kg*ng/mL/mg) | (hr) | (hr) | (hr*ng/mL) | (hr*ng/mL) | ( [symbol] *kg*ng/mL/mg) | (hr) |
| R-MDMA | 10 | 1140 | 114 | 3.5 | 11 | 4160 | 4200 | 420 | 1.01 |
| R-MDMA | 20 | 2550 | 127 | [symbol] .50 | 11 | 11300 | 11800 | 590 | 1. [symbol] 6 |

[symbol]

indicates data missing or illegible when filed

TABLE 17
S-MDMA Pharmacokinetic Parameters Following QID SC inj. of 1.25, 2.5 and 5 mg/kg/dose
(total daily doses of 5, 10, and 20 mg/kg/day) S-MDMA to Male Rats.

| | Dose | C [symbol] | C [symbol] /Dose | T [symbol] | T [symbol] | AUC [symbol] | AUC [symbol] | AUC [symbol] /Dose | T_{1/2} |
|---|---|---|---|---|---|---|---|---|---|
| Analyte | (mg/kg/day) | (ng/mL) | (kg*ng/mL/mg) | (hr) | (hr) | (hr*ng/mL) | (hr*ng/mL) | ( [symbol] *kg*ng/mL/mg) | (hr) |
| S-MDMA | 5 | 447 | 89.3 | 2.5 | 7 | 1490 | 1510 | 302 | 0.754 |
| S-MDMA | 10 | 1220 | 122 | 3.5 | 11 | 4030 | 4130 | 413 | 1.15 |
| S-MDMA | 20 | 2560 | 128 | 4 | 27 | 15000 | 15000 | 748 | 3.17 |

[symbol]

indicates data missing or illegible when filed

TABLE 18
Total MDMA Pharmacokinetic Parameters Following QID SC inj. of 2.5 and 5 mg/kg/dose
(total daily doses of 10 and 20 mg/kg/day) Rac-MDMA to Male Rats.

| | Dose | C [symbol] | C [symbol] /Dose | T [symbol] | T [symbol] | AUC [symbol] | AUC [symbol] | AUC [symbol] /Dose | T_{1/2} |
|---|---|---|---|---|---|---|---|---|---|
| Analyte | (mg/kg/day) | (ng/mL) | (kg*ng/mL/mg) | (hr) | (hr) | (hr*ng/mL) | (hr*ng/mL) | ( [symbol] *kg*ng/mL/mg) | (hr) |
| total MDMA | 10 | 1450 | 145 | 3.5 | 11 | 4710 | 4830 | 483 | 1.13 |
| total MDMA | 20 | 2700 | 135 | 3.5 | 11 | 13000 | 13600 | 682 | 1.15 |

[symbol]

indicates data missing or illegible when filed

TABLE 19
R-MDA Pharmacokinetic Parameters Following QID SC inj. of 2.5 and 5 mg/kg/dose
(total daily doses of 10 and 20 mg/kg/day) R-MDMA to Male Rats.

| | Dose | C [symbol] | C [symbol] /Dose | T [symbol] | T [symbol] | AUC [symbol] | AUC [symbol] | AUC [symbol] /Dose | | T_{1/2} |
|---|---|---|---|---|---|---|---|---|---|---|
| Analyte | (mg/kg/day) | (ng/mL) | (kg*ng/mL/mg) | (hr) | (hr) | (hr*ng/mL) | (hr*ng/mL) | ( [symbol] *kg*ng/mL/mg) | M:P^{a} | (hr) |
| R-MDA | 10 | 127 | 12.7 | 5 | 11 | 716 | 813 | 81.3 | 0.194 | NA^{b} |
| R-MDA | 20 | 272 | 13.6 | 5 | 11 | 1810 | 2410 | 121 | 0.204 | 2.67 |

NA—Not applicable.
^{a}M:P [symbol] AUC [symbol] + AUC [symbol]
^{b}T_{1/2} not reported due to insufficient data. [symbol]
indicates data missing or illegible when filed

TABLE 20
S-MDA Pharmacokinetic Parameters Following QID SC inj. of 1.25, 2.5 and 5 mg/kg/dose
(total daily doses of 5, 10, and 20 mg/kg/day) S-MDMA to Male Rats.

| | Dose | C [symbol] | C [symbol] /Dose | T [symbol] | T [symbol] | AUC [symbol] | AUC [symbol] | AUC [symbol] /Dose | | T_{1/2} |
|---|---|---|---|---|---|---|---|---|---|---|
| Analyte | (mg/kg/day) | (ng/mL) | (kg*ng/mL/mg) | (hr) | (hr) | (hr*ng/mL) | (hr*ng/mL) | ( [symbol] *kg*ng/mL/mg) | M:P^{a} | (hr) |
| S-MDA | 5 | 342 | 68.4 | 4 | 9 | 1270 | 1280 | 256 | 0. [symbol] 48 | NA^{b} |
| S-MDA | 10 | 957 | 95.7 | 5 | 11 | 5180 | 6770 | 677 | 1.64 | NA^{b} |
| S-MDA | 20 | 2500 | 125 | 5 | 27 | 26300 | 26300 | 1320 | 1.75 | NA^{b} |

NA—Not applicable.
^{a}M:P [symbol] AUC [symbol] + AUC [symbol]
^{b}T_{1/2} not reported due to insufficient data. [symbol]
indicates data missing or illegible when filed

TABLE 21
Total MDA Pharmacokinetic Parameters Following QID SC inj. of 2.5 and 5 mg/kg/dose
(total daily doses of 10 and 20 mg/kg/day) Rac-MDMA to Male Rats.

| | Dose | C [symbol] | C [symbol] /Dose | T [symbol] | T [symbol] | AUC [symbol] | AUC [symbol] | AUC [symbol] /Dose | | T_{1/2} |
|---|---|---|---|---|---|---|---|---|---|---|
| Analyte | (mg/kg/day) | (ng/mL) | (kg*ng/mL/mg) | (hr) | (hr) | (hr*ng/mL) | (hr*ng/mL) | ( [symbol] *kg*ng/mL/mg) | M:P^{a} | (hr) |
| total MDA | 10 | 546 | 54.6 | 4 | 11 | 3240 | 4340 | 434 | 0.89 [symbol] | 3.00 |
| total MDA | 20 | 1290 | 64.5 | 5 | 11 | 7540 | 9020 | 451 | 0.662 | NA^{b} |

NA—Not applicable.
^{a}M:P [symbol] AUC [symbol] + AUC [symbol]
^{b}T_{1/2} not reported due to insufficient data. [symbol]
indicates data missing or illegible when filed

Conclusions from plasma analyses.

R-MDMA—Following Doses of 10 and 20 mg/kg/Day

Following QID subcutaneous injection of R-MDMA, Cₘₐₓ and AUC_{0-27 hr} values for R-MDMA increased with increasing dose from 10 to 20 mg/kg/day in an approximately dose proportional manner.

R-MDA—Following Doses of 10 and 20 mg/kg/Day

Following QID subcutaneous injection of R-MDMA, Cₘₐₓ and AUC_{0-27 hr} values for R-MDA increased with increasing dose from 10 to 20 mg/kg/day in an approximately dose proportional manner.

Systemic exposure (AUC_{0-27 hr} values) to R-MDA was approximately 20% of systemic exposure to R-MDMA.

S-MDMA—Following Doses of 5, 10, and 20 mg/kg/Day

Following QID subcutaneous injection of S-MDMA, Cₘₐₓ and AUC_{0-27 hr} values for S-MDMA increased with increasing dose from 5 to 20 mg/kg/day in an approximately dose proportional manner from 5 to 10 mg/kg/day and in a greater than dose proportional manner from 5 to 20 mg/kg/day.

S-MDA—Following Doses of 5, 10, and 20 mg/kg/Day

Following QID subcutaneous injection of S-MDMA, Cₘₐₓ and AUC_{0-27 hr} values for S-MDA increased with increasing dose from 5 to 20 mg/kg/day in an approximately dose proportional manner from 5 to 10 mg/kg/day and in a greater than dose proportional manner from 5 to 20 mg/kg/day.

Systemic exposure (AUC_{0-27 hr} values) to S-MDA appeared similar at 5 mg/kg/day and was approximately 1.64-fold and 1.75-fold greater than systemic exposure to S-MDMA. S-MDA:S-MDMA ratios, based on AUC₀₋₂₇ hr, were 0.848, 1.64, and 1.75 at 5, 10, and 20 mg/kg/day.

Total MDMA—Following Doses of 10 and 20 mg/kg/Day of Racemic-MDMA

Following QID subcutaneous injection of Racemic-MDMA, Cₘₐₓ and AUC_{0-27 hr} values for total MDMA increased with increasing dose from 10 to 20 mg/kg/day in an approximately dose proportional manner.

Total MDA—Following Doses of 10 and 20 mg/kg/Day of Racemic-MDMA

Following QID subcutaneous injection of Racemic-MDMA, Cₘₐₓ and AUC_{0-27 hr} values for total MDA increased with increasing dose from 10 to 20 mg/kg/day in an approximately dose proportional manner.

Systemic exposure (AUC_{0-27 hr} values) to total MDA appeared similar to the systemic exposure of total MDMA. Total MDA:Total MDMA ratios, based on AUC₀₋₂₇ hr, were 0.898 and 0.662 at 10 and 20 mg/kg/day

Brain: The results from calibration standards and quality control samples demonstrated acceptable performance of the method for all reported concentrations. The Tables below (22-25) summarize the frontal cortex 5-HT and 5-HIAA and striatal DA, DOPAC, and HVA concentrations 1 week following cumulative SC administration of R-MDMA (R-MDMA), S-MDMA or racemic MDMA.

TABLE 22

| | | | Mean Brain Concentrations (ng/g) | | | | |
|---|---|---|---|---|---|---|---|
| | | | 5-HIAA | 5-HT | DA | DOPAC | HVA |
| R-MDMA | 0 | Mean | 453.38 | 152.46 | 4070.16 | 1555.00 | 554.75 |
| | mg/kg | SD | 84.046 | 66.951 | 3797.108 | 967.205 | 351.192 |
| | | N | 8 | 8 | 8 | 8 | 8 |
| | 2.5 | Mean | 389.83 | 226.00 | 6463.67 | 1720.17 | 633.50 |
| | mg/kg | SD | 71.586 | 100.469 | 4146.525 | 695.025 | 213.608 |
| | | N | 6 | 6 | 6 | 6 | 6 |
| | 5 | Mean | 318.13 | 172.90 | 8803.75 | 1870.00 | 804.00 |
| | mg/kg | SD | 50.138 | 47.089 | 3337.176 | 431.906 | 212.386 |
| | | N | 8 | 8 | 8 | 8 | 8 |
| | 10 | Mean | 305.50 | 41.60 | 321.00 | 266.00 | 60.50 |
| | mg/kg | SD | 17.678 | 51.760 | 274.357 | 87.681 | 71.418 |
| | | N | 2 | 2 | 2 | 2 | 2 |
| S-MDMA | 0 | Mean | 353.88 | 192.28 | 7620.38 | 1762.13 | 685.00 |
| | mg/kg | SD | 95.278 | 78.543 | 3537.035 | 828.671 | 299.024 |
| | | N | 8 | 8 | 8 | 8 | 8 |
| | 1.5 | Mean | 417.50 | 170.88 | 9975.00 | 1965.00 | 872.75 |
| | mg/kg | SD | 105.041 | 107.736 | 1328.872 | 258.005 | 196.790 |
| | | N | 4 | 4 | 4 | 4 | 4 |
| | 2.5 | Mean | 251.83 | 121.65 | 7733.33 | 1883.33 | 716.67 |
| | mg/kg | SD | 127.559 | 77.396 | 3845.921 | 574.062 | 175.953 |
| | | N | 6 | 6 | 6 | 6 | 6 |
| | 5 | Mean | 93.25 | 20.08 | 2142.00 | 548.00 | 319.25 |
| | mg/kg | SD | 45.795 | 30.150 | 2710.099 | 475.102 | 253.355 |
| | | N | 4 | 4 | 4 | 4 | 4 |
| | 10 | Mean | 127.00 | 80.60 | 2945.00 | 725.00 | 757.50 |
| | mg/kg | SD | 19.799 | 8.202 | 176.777 | 296.985 | 64.347 |
| | | N | 2 | 2 | 2 | 2 | 2 |
| Racemic | 0 | Mean | 440.75 | 190.61 | 5264.78 | 1425.75 | 533.13 |
| MDMA | mg/kg | SD | 76.380 | 99.818 | 3519.298 | 756.618 | 324.621 |
| | | N | 8 | 8 | 8 | 8 | 8 |
| | 2.5 | Mean | 368.17 | 183.90 | 5956.67 | 1811.00 | 706.83 |
| | mg/kg | SD | 147.238 | 116.721 | 2964.083 | 979.730 | 321.470 |
| | | N | 6 | 6 | 6 | 6 | 6 |
| | 5 | Mean | 234.63 | 88.38 | 5190.63 | 1962.75 | 577.00 |
| | mg/kg | SD | 116.986 | 44.982 | 2007.006 | 842.536 | 174.674 |
| | | N | 8 | 8 | 8 | 8 | 8 |
| | 10 | Mean | 235.00 | 100.85 | 7405.00 | 2015.00 | 923.50 |
| | mg/kg | SD | 127.279 | 28.496 | 403.051 | 1279.863 | 81.317 |
| | | N | 2 | 2 | 2 | 2 | 2 |
| R-MDMA, | 0 | Mean | 416 | 178.45 | 5651.77 | 1580.96 | 590.96 |
| S-MDMA-, | mg/kg | SD | 93.432 | 81.407 | 3772.97 | 829.438 | 318.618 |
| Rac-MDMA | | N | 24 | 24 | 24 | 24 | 24 |
| R-MDMA, | 0 | Mean | 447.06 | 171.54 | 4667.47 | 1490.38 | 543.94 |
| Rac-MDMA | mg/kg | SD | 77.855 | 84.437 | 3590.105 | 841.527 | 326.892 |
| | | N | 16 | 16 | 16 | 16 | 16 |
| S-MDMA-, | 0 | Mean | 397.31 | 191.44 | 6442.58 | 1593.94 | 609.06 |
| Rac-MDMA | mg/kg | SD | 94.718 | 86.772 | 3619.097 | 785.992 | 311.536 |
| | | N | 16 | 16 | 16 | 16 | 16 |

TABLE 23
R-MDMA Brain monoamines and metabolites statistical analyses.

| | | | Concentrations (ng/g) | | |
|---|---|---|---|---|---|
| Sex: Male | | | 5-HIAA | 5-HT | DA |
| R- | 0 | Mean | 453.38 | 152.46 | 4070.16(c* f*) |
| MDMA | mg/kg | SD | 84.046 | 66.951 | 3797.108 |
| | | N | 8 | 8 | 8 |
| | 2.5 | Mean | 389.83 | 226.00(d* l**) | 6463.67 |
| | mg/kg | SD | 71.586 | 100.469 | 4146.525 |
| | | N | 6 | 6 | 6 |
| | 5 | Mean | 318.13 | 172.90(h**) | 8803.75(a* d** h** o*) |
| | mg/kg | SD | 50.138 | 47.089 | 3337.176 |
| | | N | 8 | 8 | 8 |
| | 10 | Mean | 305.50 | 41.60(b*) | 321.00(c** e*) |
| | mg/kg | SD | 17.678 | 51.760 | 274.357 |
| | | N | 2 | 2 | 2 |

| | | | Concentrations (ng/g) | |
|---|---|---|---|---|
| Sex: Male | | | DOPAC | HVA |
| R- | 0 | Mean | 1555.00 | 554.75 |
| MDMA | mg/kg | SD | 967.205 | 351.192 |
| | | N | 8 | 8 |
| | 2.5 | Mean | 1720.17 | 633.50(d*) |
| | mg/kg | SD | 695.025 | 213.608 |
| | | N | 6 | 6 |
| | 5 | Mean | 1870.00(d* h*) | 804.00(d** h**) |
| | mg/kg | SD | 431.906 | 212.386 |
| | | N | 8 | 8 |
| | 10 | Mean | 266.00(c*) | 60.50(b*c**e* l*m**n*o*) |
| | mg/kg | SD | 87.681 | 71.418 |
| | | N | 2 | 2 |

(*)= p ≤ 0.05;
(**)= p ≤ 0.01
(a)differs from R-MDMA at 0 mg/kg
(b)differs from R-MDMA at 2.5 mg/kg
(c)differs from R-MDMA at 5 mg/kg
(d)differs from R-MDMA at 10 mg/kg
(e)differs from S-MDMA at 0 mg/kg
(f)differs from S-MDMA at 1.25 mg/kg
(h)differs from S-MDMA at 5 mg/kg
i - differs from S-MDMA at 10 mg/kg
(l)differs from racemic-MDMA at 5 mg/kg
(m)differs from racemic-MDMA at 10 mg/kg
(n)differs from pooled R-MDMA, S-MDMA, racemic-MDMA at 0 mg/kg
(o)differs from pooled R-MDMA, racemic-MDMA at 0 mg/kg

TABLE 24
S-MDMA Brain monoamines and metabolites statistical analyses:

| | | | Concentrations (ng/g) | | |
|---|---|---|---|---|---|
| Sex: Male | | | 5-HIAA | 5-HT | DA |
| S-MDMA | 0 | Mean | 353.88 | 192.28 ₕ** | 7620.38 _{d}* |
| | mg/kg | SD | 95.278 | 78.543 | 3537.035 |
| | | N | 8 | 8 | 8 |
| | 1.5 | Mean | 417.50 | 170.88 ₕ* | 9975.00 ₐ*ₕ** |
| | mg/kg | SD | 105.041 | 107.736 | 1328.872 |
| | | N | 4 | 4 | 4 |
| | 2.5 | Mean | 251.83 | 121.65 | 7733.33 ₕ* |
| | mg/kg | SD | 127.559 | 77.396 | 3845.921 |
| | | N | 6 | 6 | 6 |
| | 5 | Mean | 93.25 | 20.08 _{c}**ₑ**_{f}*ⱼ**ₙ**ₚ** | 2142.00 _{c}**_{f}** _{g}* |
| | mg/kg | SD | 45.795 | 30.150 | 2710.099 |
| | | N | 4 | 4 | 4 |
| | 10 | Mean | 127.00 | 80.60 | 2945.00 |
| | mg/kg | SD | 19.799 | 8.202 | 176.777 |
| | | N | 2 | 2 | 2 |

| | | | Concentrations (ng/g) | |
|---|---|---|---|---|
| Sex: Male | | | DOPAC | HVA |
| S-MDMA | 0 | Mean | 1762.13 | 685.00 _{d}* |
| | mg/kg | SD | 828.671 | 299.024 |
| | | N | 8 | 8 |
| | 1.5 | Mean | 1965.00 ₕ* | 872.75 ₕ* |
| | mg/kg | SD | 258.005 | 196.790 |
| | | N | 4 | 4 |
| | 2.5 | Mean | 1883.33 ₕ* | 716.67 |
| | mg/kg | SD | 574.062 | 175.953 |
| | | N | 6 | 6 |
| | 5 | Mean | 548.00 _{c}*_{f}*_{g}*ₗ** ₘ* | 319.25 _{c}**_{f}*ₘ* |
| | mg/kg | SD | 475.102 | 253.355 |
| | | N | 4 | 4 |
| | 10 | Mean | 725.00 | 757.50 _{d}* |
| | mg/kg | SD | 296.985 | 64.347 |
| | | N | 2 | 2 |

* = p ≤ 0.05;
** = p ≤ 0.01
ₐ differs from R-MDMA at 0 mg/kg
_{c} differs from R-MDMA at 5 mg/kg
_{d} differs from R-MDMA at 10 mg/kg
ₑ differs from S-MDMA at 0 mg/kg
_{f} differs from S-MDMA at 1.25 mg/kg
_{g} differs from S-MDMA at 2.5 mg/kg
ₕ differs from S-MDMA at 5 mg/kg
ⱼ differs from racemic-MDMA at 0 mg/kg
ₗ differs from racemic-MDMA at 5 mg/kg
ₘ differs from racemic-MDMA at 10 mg/kg
ₙ differs from pooled R-MDMA, S-MDMA, racemic-MDMA at 0 mg/kg
ₚ differs from pooled S-MDMA, racemic-MDMA at 0 mg/kg

TABLE 25
Racemic MDMA Brain monoamines and metabolites statistical analyses:

| | | | Concentrations (ng/g) | | | | |
|---|---|---|---|---|---|---|---|
| Sex: Male | | | 5-HIAA | 5-HT | DA | DOPAC | HVA |
| Racemic | 0 | Mean | 440.75 | 190.61 h** | 5264.78 | 1425.75 | 533.13 |
| MDMA | mg/kg | SD | 76.380 | 99.818 | 3519.298 | 756.618 | 324.621 |
| | | N | 8 | 8 | 8 | 8 | 8 |
| | 2.5 | Mean | 368.17 | 183.90 | 5956.67 | 1811.00 | 706.83 |
| | mg/kg | SD | 147.238 | 116.721 | 2964.083 | 979.730 | 321.470 |
| | | N | 6 | 6 | 6 | 6 | 6 |
| | 5 | Mean | 234.63 | 88.38 b** n* p* | 5190.63 | 1962.75 h** | 577.00 |
| | mg/kg | SD | 116.986 | 44.982 | 2007.006 | 842.536 | 174.674 |
| | | N | 8 | 8 | 8 | 8 | 8 |
| | 10 | Mean | 235.00 | 100.85 | 7405.00 | 2015.00 h* | 923.50 d**h* |
| | mg/kg | SD | 127.279 | 28.496 | 403.051 | 1279.863 | 81.317 |
| | | N | 2 | 2 | 2 | 2 | 2 |

* = p ≤ 0.05;
** = p ≤ 0.01
b differs from R-MDMA at 2.5 mg/kg
d differs from R-MDMA at 10 mg/kg
h differs from S-MDMA at 5 mg/kg
n differs from pooled R-MDMA, S-MDMA, racemic-MDMA at 0 mg/kg
p differs from pooled S-MDMA, racemic-MDMA at 0 mg/kg

Conclusions from Brain Analyses

R-MDMA: Compared to the corresponding vehicle condition (n=8), cumulative SC administration of R-MDMA at 5 mg/kg/injection significantly increased striatal DA. Compared to the pooled vehicle condition (n=24), R-MDMA at 10 mg/kg/injection significantly reduced striatal HVA, but the limited n=2 suggests this finding should be interpreted with caution.

S-MDMA: Compared to the corresponding vehicle condition (n=8) and the pooled vehicle condition (n=24), cumulative SC administration of S-MDMA at 5 mg/kg/injection significantly decreased frontal cortex 5-HT concentrations.

Racemic MDMA: Compared to the pooled vehicle condition (n=24), cumulative SC administration of racemic MDMA at 5 mg/kg/injection significantly reduced frontal cortex 5-HT concentrations.

Racemic MDMA has been reported to show efficacy in a Phase 3 clinical trial for PTSD (Mitchell et al., 2021). That study reported that a transient increase in vital signs (systolic and diastolic blood pressure and heart rate) was observed in the MDMA group. Two participants in the MDMA group had a transient increase in body temperature to 38.1° C.: one had an increase after the second MDMA session, and one had an increase after the second and third MDMA sessions.

A report by Biezonski et al., 2013, showed significant reductions in frontal cortical 5-HT and 5-HIAA and striatal DOPAC and HVA concentrations in rats 1 week following cumulative SC administration of 4 hourly racemic MDMA injections at 10 mg/kg/injection. Consistent with this publication, our data indicates a significant reduction in frontal cortical 5-HT levels 1 week following cumulative SC administration of racemic MDMA at 5 mg/kg/injection, an effect that also was observed following cumulative SC administration of S-MDMA at 5 mg/kg/injection. Depletion in regional 5-HT and 5-HIAA has been reported in the literature previously and interpreted as an indication of the presence of serotonergic neurotoxicity known to occur following racemic MDMA exposure in rats (Green et al., 2003). These results suggested that cumulative SC administration of mass-equivalent 5 mg/kg/day S-MDMA and racemic-MDMA exhibited greater potential for changes in the frontal cortical serotonergic system than R-MDMA under the noted conditions of the present study.

In the mouse locomotor activity assay described herein, single administration of R-MDMA exhibited a limited magnitude and duration of locomotor activation up to 30 mg/kg IP. In the same assay, single administration of a 7.8 mg/kg IP reference dose of racemic MDMA showed a greater magnitude and duration of locomotor activation.

In our rat safety pharmacology studies that included telemetry measures of heart rate, blood pressure, body temperature and activity, an enantiomeric equivalent cumulative SC dose of R-MDMA (10 mg/kg/day) exhibited lower magnitude and/or reduced duration of effects compared to S-MDMA (10 mg/kg/day) and/or racemic MDMA (20 mg/kg/day). In addition, a cumulative SC dose of R-MDMA (5 mg/kg/day) did not reduce serotonin content in the frontal cortex of rats 1 week following dosing, while cumulative SC doses of an enantiomeric equivalent dose of S-MDMA (5 mg/kg/day) and an enantiomeric lower dose of racemic MDMA (5 mg/kg/day=2.5 mg/kg/day R-MDMA+2.5 mg/kg/day S-MDMA) reduced serotonin content in the rat frontal cortex 1 week following dosing.

The data included in the Examples above are believed to be the first comparisons of the MDMA racemate to the single MDMA enantiomer(s) under the reported test conditions (i.e., mouse locomotor activity evaluated over 11 days post single administration; rat heart rate, blood pressure, body temperature and activity following cumulative SC administration; rat frontal cortex 5-HT concentrations at 1 week following cumulative SC administration). The data suggest that the R-MDMA enantiomer exhibits lower potency and/or efficacy to produce the measured behavioral, physiological and neurochemical effects than either racemic or S-MDMA. As the measured effects may be considered undesirable, the lower potency/effect of R-MDMA on these endpoints indicate a likely superior safety profile for R-MDMA compared to prior reports for compositions that have included racemic- or S-MDMA.

Although the invention has been described with reference to the above examples, it will be understood that modifications and variations are encompassed within the spirit and scope of the invention. Accordingly, the invention is limited only by the following claims.

What is claimed is:

1. A method of treating a stress-related disease or disorder in a subject comprising administering to the subject a therapeutically effective amount of a composition comprising R(−)-3,4-methylenedioxymethamphetamine (R(−)-MDMA) to lessen avoidance behavior,
thereby treating the stress-related disease or disorder in the subject.

2. The method of claim 1, wherein the stress-related disease or disorder is mood/depressive disorder, bipolar disorder, anxiety disorder, psychotic or delirium disorder, schizophrenia, schizoaffective disorder, personality disorder, abuse or neglect disorder, tic disorder, neurocognitive disorder, neurodevelopmental disorder, learning disorder, disruptive mood regulation disorder, intermittent explosive disorder, antisocial personality disorder, conduct disorder, behavioral and psychological symptoms of dementia, depression, treatment resistant depression, anxiety, post-traumatic stress disorder (PTSD), or any combination thereof.

3. The method of claim 1, wherein the stress-related disease or disorder is selected from the group consisting of depression, anxiety, post-traumatic stress disorder (PTSD), and any combination thereof.

4. The method of claim 1, wherein R(−)-MDMA has antidepressant and anxiolytic effects.

5. The method of claim 1, wherein said therapeutically effective amount comprises between about 1 mg/kg and 20 mg/kg R(−)-MDMA.

6. The method of claim 1, wherein said therapeutically effective amount comprises between about 25 mg and 350 mg R(−)-MDMA.

7. The method of claim 1, wherein said therapeutically effective amount comprises about 5 mg/kg R(−)-MDMA.

8. The method of claim 1, wherein the administering comprises intracutaneous, subcutaneous, intravenous, intraarterial, intradermal, transdermal, oral, sublingual, buccal, or nasal route of administration.

9. The method of claim 1, comprising administering R(−)-MDMA as a single dose.

10. The method of claim 1, comprising administering R(−)-MDMA in repeated doses.

11. The method of claim 1, further comprising administering a therapeutic agent.

12. The method of claim 11, wherein the therapeutic agent is a selective serotonin reuptake inhibitor (SSRI).

13. The method of claim 12, wherein the SSRI is fluoxetine, paroxetine, sertraline, escitalopram or citalopram.

14. The method of claim 13, comprising administering the therapeutic agent prior to, concurrently with or after R(−)-MDMA.

15. The method of claim 1, wherein the subject is also undergoing psychotherapy treatment.

16. The method of claim 15, wherein the psychotherapy treatment is cognitive processing therapy (CPT), cognitive behavioral therapy (CBT), prolonged exposure therapy (PET), brief eclectic psychotherapy (BEP), narrative exposure therapy (NAT), or eye-movement desensitization and reprocessing (EMDR).

17. A method of activating 5-HT_{2A} and 5-HT_{2C} receptors in a subject comprising administering to the subject a composition comprising an effective amount of R(−)-3,4-methylenedioxymethamphetamine (R(−)-MDMA),
thereby activating 5-HT_{2A} and 5-HT_{2C} receptors in the subject.

18. The method of claim 17, wherein R(−)-MDMA is a partial agonist of 5-HT_{2A}.

19. The method of claim 17, wherein R(−)-MDMA induces neurite growth.

20. A method of decreasing side effects of 3,4-methylenedioxy-methamphetamine (MDMA) treatment comprising administering to the subject a therapeutically effective amount of a composition comprising R(−)-3,4-methylenedioxymethamphetamine (R(−)-MDMA) to lessen avoidance behavior in the subject,
thereby decreasing side effects of MDMA treatment.

21. The method of claim 20, wherein the subject has a stress-related disease or disorder.

22. The method of claim 21, wherein the stress-related disease or disorder is mood/depressive disorder, bipolar disorder, anxiety disorder, psychotic or delirium disorder, schizophrenia, schizoaffective disorder, personality disorder, abuse or neglect disorder, tic disorder, neurocognitive disorder, neurodevelopmental disorder, learning disorder, disruptive mood regulation disorder, intermittent explosive disorder, antisocial personality disorder, conduct disorder, behavioral and psychological symptoms of dementia, depression, treatment resistant depression, anxiety, post-traumatic stress disorder (PTSD), or any combination thereof.

23. The method of claim 22, wherein the stress-related disease or disorder is PTSD.

24. The method of claim 20, wherein the side effects are cardiovascular effects, hyperthermia, neurotoxicity or a combination thereof.

25. The method of claim 24, wherein the cardiovascular effects are increased blood pressure, increased heart rate or a combination thereof.

26. The method of claim 24, wherein neurotoxicity comprises mood disorder, cognition disorder and/or psychomotor deficits.

27. The method of claim 20, wherein R(−)-MDMA has antidepressant and anxiolytic effects.

28. The method of claim 20, wherein said therapeutically effective amount comprises between about 1 mg/kg and 20 mg/kg R(−)-MDMA.

29. The method of claim 19, wherein the administering comprises intracutaneous, subcutaneous, intravenous, intraarterial, intradermal, transdermal, oral, sublingual, buccal, or nasal route of administration.

30. A method for inducing neurite outgrowth in a subject comprising administering to the subject a therapeutically effective amount of a composition comprising R(−)-3,4-methylenedioxymethamphetamine (R(−)-MDMA) to lessen avoidance behavior in the subject,
thereby inducing neurite outgrowth in the subject.

31. The method of claim 30, wherein neurite outgrowth comprises neurite number, neurite total length, number of neurite branch points per neuron or any combination thereof.

32. The method of claim 30, wherein the neurite outgrowth comprises neurite outgrowth on prefrontal cortex neurons and/or hippocampal neurons.

33. The method of claim 30, wherein said therapeutically effective amount comprises between about 1 mg/kg and 20 mg/kg R(−)-MDMA.

34. The method of claim 30, wherein the subject has a stress-related disease or disorder.

35. The method of claim 34, wherein the stress-related disease or disorder is mood/depressive disorder, bipolar disorder, anxiety disorder, psychotic or delirium disorder, schizophrenia, schizoaffective disorder, personality disorder, abuse or neglect disorder, tic disorder, neurocognitive disorder, neurodevelopmental disorder, learning disorder, disruptive mood regulation disorder, intermittent explosive disorder, antisocial personality disorder, conduct disorder, behavioral and psychological symptoms of dementia, depression, treatment resistant depression, anxiety, post-traumatic stress disorder (PTSD) or any combination thereof.

36. The method of claim 35, wherein the stress-related disease or disorder is PTSD.

37. A method of treating neuronal atrophy in a subject comprising administering to the subject a therapeutically effective amount of a composition comprising R(−)-3,4-methylenedioxymethamphetamine (R(−)-MDMA) to lessen avoidance behavior in the subject,
thereby treating neuronal atrophy in the subject.

38. The method of claim 37, wherein administration of R(−)-MDMA induces neurite outgrowth.

39. The method of claim 38, wherein neurite outgrowth comprises increasing neurite number, neurite total length, number of neurite branch points per neuron or any combination thereof.

40. The method of claim 38, wherein the neurite outgrowth comprises neurite outgrowth on prefrontal cortex neurons and/or hippocampal neurons.

41. The method of claim 37, wherein said therapeutically effective amount comprises between about 1 mg/kg and 20 mg/kg R(−)-MDMA.

42. The method of claim 37, wherein the subject has a stress related disorder.

43. The method of claim 42, wherein the stress-related disease or disorder is mood/depressive disorder, bipolar disorder, anxiety disorder, psychotic or delirium disorder, schizophrenia, schizoaffective disorder, personality disorder, abuse or neglect disorder, tic disorder, neurocognitive disorder, neurodevelopmental disorder, learning disorder, disruptive mood regulation disorder, intermittent explosive disorder, antisocial personality disorder, conduct disorder, behavioral and psychological symptoms of dementia, depression, treatment resistant depression, anxiety, post-traumatic stress disorder (PTSD) or any combination thereof or any combination thereof.

44. The method of claim 43, wherein the stress-related disease or disorder is PTSD.

45. A method of inducing structural neuroplasticity in a subject comprising administering to the subject a therapeutically effective amount of a composition comprising R(−)-3,4-methylenedioxymethamphetamine (R(−)-MDMA),
thereby inducing structural neuroplasticity in the subject.

46. The method of claim 45, wherein administration of R(−)-MDMA induces neurite outgrowth.

47. The method of claim 46, wherein neurite outgrowth comprises increasing neurite number, neurite total length, number of neurite branch points per neuron or a combination thereof.

48. The method of claim 46, wherein the neurite outgrowth comprises neurite outgrowth on prefrontal cortex neurons and/or hippocampal neurons.

49. The method of claim 45, wherein said therapeutically effective amount comprises between about 1 mg/kg and 20 mg/kg R(−)-MDMA.

50. The method of claim 52, wherein the subject has a stress-related disease or disorder.

51. The method of claim 50, wherein the stress-related disease or disorder is mood/depressive disorder, bipolar disorder, anxiety disorder, psychotic or delirium disorder, schizophrenia, schizoaffective disorder, personality disorder, abuse or neglect disorder, tic disorder, neurocognitive disorder, neurodevelopmental disorder, learning disorder, disruptive mood regulation disorder, intermittent explosive disorder, antisocial personality disorder, conduct disorder, behavioral and psychological symptoms of dementia, depression, treatment resistant depression, anxiety, post-traumatic stress disorder (PTSD) or any combination thereof.

52. The method of claim 51, wherein the stress-related disease or disorder is PTSD.

53. A method of increasing brain-derived neurotrophic factor (BDNF) levels in a subject comprising administering to the subject a therapeutically effective amount of a composition comprising R(−)-3,4-methylenedioxymethamphetamine (R(−)-MDMA) to lessen avoidance behavior,
thereby increasing BDNF levels in the subject.

54. The method of claim 53, wherein increasing BDNF levels comprises increasing cerebral cortex and hippocampal BDNF levels.

55. The method of claim 53, wherein increasing BDNF levels increases neuronal survival and synaptic plasticity.

56. The method of claim 53, wherein said therapeutically effective amount comprises between about 1 mg/kg and 20 mg/kg R(−)-MDMA.

57. The method of claim 53, wherein the subject has a stress-related disease or disorder.

58. The method of claim 57, wherein the stress-related disease or disorder is mood/depressive disorder, bipolar disorder, anxiety disorder, psychotic or delirium disorder, schizophrenia, schizoaffective disorder, personality disorder, abuse or neglect disorder, tic disorder, neurocognitive disorder, neurodevelopmental disorder, learning disorder, disruptive mood regulation disorder, intermittent explosive disorder, antisocial personality disorder, conduct disorder, behavioral and psychological symptoms of dementia, depression, treatment resistant depression, anxiety, post-traumatic stress disorder (PTSD) or any combination thereof.

59. The method of claim 58, wherein the stress-related disease or disorder is PTSD.

60. A pharmaceutical composition comprising R(−)-MDMA and a pharmaceutically acceptable carrier.

61. The pharmaceutical composition of claim 52, wherein the pharmaceutically acceptable carrier is saline or purified water.
